(19) United States
(12) Patent Application Publication
(10) Pub. No.: US 2024/0271133 A1
(43) Pub. Date: Aug. 15, 2024

(54) KRAB-Containing Zinc Finger Protein And Cancer

(71) Applicant: ECOLE POLYTECHNIQUE FEDERALE DE LAUSANNE (EPFL), Lausanne (CH)

(72) Inventors: Didier TRONO, Vufflens-Chateau (CH); Filipe MARTINS, Chene-Bougeries (CH); Priscilla TURELLI, Echandens-Denges (CH)

(21) Appl. No.: 18/289,973

(22) PCT Filed: May 12, 2022
(86) PCT No.: PCT/EP2022/062972

§ 371 Date: Nov. 8, 2023

(30) Foreign Application Priority Data
May 12, 2021 (WO) PCT/EP2021/062606

(51) Int. Cl. C12N 15/113; C12Q 1/6869; C12Q 1/6886

(52) U.S. Cl. CPC C12N 15/113; C12Q 1/6869; C12Q 1/6886; C12N 2310/14; C12N 2310/531; C12Q 2600/158

(57) ABSTRACT

The present invention provides methods for detecting cancer as well as agents and compositions for treating cancer by modulating the expression and/or activity of one or more i) KRAB-containing zinc finger protein (KZFP), ii) mRNA encoding a KZFP, and/or iii) KZFP gene.

### FIELD OF THE INVENTION

The present invention provides methods for detecting cancer as well as agents and compositions for treating cancer by modulating the expression and/or activity of one or more i) KRAB-containing zinc finger protein (KZFP), ii) mRNA encoding a KZFP, and/or iii) KZFP gene.

### SEQUENCE LISTING

The instant application contains a Sequence Listing, named PAT7576PC01_ST25, which has been submitted electronically in ASCII format and is hereby incorporated by reference in its entirety.

### BACKGROUND OF THE INVENTION

DLBCL (diffuse large B cell lymphoma) is the most frequently diagnosed aggressive non-Hodgkin lymphoma (NHL)¹. It comprises several histological variants which can be grouped into three main molecular subtypes based on gene expression signatures, namely germinal center (GCB), activated B-cell (ABC) and unclassified (UNCL) DLBCL². The distinction between these three groups carries prognostic and therapeutic value. Indeed, ABC DLBCL has a worse prognosis than its GCB or UNCL counterparts as it is often refractory to first line standard treatments and prone to relapse. The standard of care for first-line therapy in DLBCL is the R-CHOP (rituximab, cyclophosphamide, doxorubicin, vincristine and prednisone) regimen.

Research on drug resistance has so far mainly focused on genetic alterations, but recent data points to the role of epigenetic changes. For instance, anthracycline-resistance has been linked to silencing of the transcription factor SMAD1 by DNA methylation of its promoter, resulting in impaired activation of DNA damage response genes³. Accordingly, inhibitors of DNA methylation, such as 5-azacytidine, are currently coupled with R-CHOP in some clinical trials⁴.

Although advances in therapy over the last 20 years have led to improvements in cancer survival, additional strategies are still urgently needed.

### SUMMARY OF THE INVENTION

The present invention provides agent modulating the expression and/or activity of one or more i) KRAB-containing zinc finger protein (KZFP), ii) mRNA encoding a KZFP, and/or iii) KZFP gene, for use in the treatment and/or prevention of cancer and/or cancer metastasis in a subject in need thereof, wherein said one or more KZFP is selected from the group comprising those listed in Table 1, or a combination of one or more thereof.

The present invention further provides a plasmid or a vector comprising one or more nucleic acid(s) encoding the siRNA, miRNA, piRNA, hnRNA, snRNA, sg RNA, CRISPR-based loss-of-function system, esiRNA, shRNA, and antisense oligonucleotide, or combination of one or more thereof, of the invention.

The present invention provides a host cell comprising, or modified by the introduction of,
i) a plasmid or vector of the invention, or
ii) one or more nucleic acid(s) encoding the siRNA, miRNA, piRNA, hnRNA, snRNA, sg RNA, CRISPR-based loss-of-function system, esiRNA, shRNA, and antisense oligonucleotide, or combination of one or more thereof, of the invention.

The present invention provides a pharmaceutical composition comprising
i) a therapeutically effective amount of an agent modulating the expression and/or activity of one or more i) KZFP, ii) mRNA encoding a KZFP and/or iii) KZFP gene, of anyone of the invention, or
ii) a plasmid or a vector of the invention, or
iii) a host cell of the invention,
and a pharmaceutically acceptable carrier or diluent.

The present invention provides method of diagnosing cancer and/or cancer metastasis in a subject comprising:
(a) detecting, directly or indirectly, and measuring the level of transcription and/or expression and/or activity of one or more KZFP in a sample obtained from said subject;
(b) comparing the level of transcription and/or expression and/or activity of said one or more KZFP to a control biological sample for the same one or more KZFP;
wherein a differential transcription and/or expression and/or activity of one or more one or more KZFP in said biological sample, relative to the level of corresponding said one or more KZFP in a control biological sample of a cancer-free subject, is indicative of the subject having cancer and/or cancer metastasis.

The present invention provides a method of determining the progression or regression of a cancer in a subject suffering therefrom and under cancer treatment, said method comprising:
(a) detecting, directly or indirectly, and measuring the level of transcription and/or expression and/or activity of one or more KZFP in a sample obtained from said subject;
(b) periodically comparing the level of transcription and/or expression and/or activity of said one or more KZFP,
wherein an alteration in the level of transcription and/or expression and/or activity of one or more KZFP in a sample obtained from the same subject, relative to the level of transcription and/or expression and/or activity of one or more KZFP, determined previously, is indicative of the progression or regression of said cancer.

The present invention provides a method of treatment of cancer, and/or cancer metastasis comprising administering an agent modulating the expression and/or activity of one or more i) KRAB-containing zinc finger protein (KZFP), ii) mRNA encoding a KZFP, and/or iii) KZFP gene, of the invention to a subject in need thereof.

The present invention provides a kit comprising
(a) one or more agents of the invention, plasmid or vector of the invention, host cell of the invention and/or a pharmaceutical composition of of the invention, and
(b) instructions for use.

The present invention provides a method of stratifying a cancer in a subject comprising:
(a) detecting, directly or indirectly, and measuring the level of transcription and/or expression and/or activity of one or more KZFP in a sample obtained from said subject;
(b) comparing the level of transcription and/or expression and/or activity of said one or more KZFP to a control biological sample for the same one or more KZFP;
wherein an alteration in the transcription and/or expression and/or activity level of one or more one or more KZFP in said biological sample, relative to the level of corresponding said one or more KZFP in a control biological sample of a cancer-free subject, is indicative of the disease stage or grade.

The present invention provides a method for classifying a cancer subject, the method comprising
(a) detecting, directly or indirectly, and measuring the level of transcription and/or expression and/or activity of one or more KZFP in a biological sample obtained from said subject, wherein said one or more KZFP is selected from the group listed in Table 1 or a variant or fragment thereof, and
(b) classifying the cancer subject as having a poor prognosis based on the presence and/or alteration of the transcription and/or expression and/or activity level(s) of the one or more KZFP in the biological sample.

The present invention provides one or more oligonucleotide probes or primers specific to one or more target sequences within an mRNA encoding a KZFP, or a combination of KZFP, selected from any of SEQ ID Nos of Table 1.

### DESCRIPTION OF THE FIGURES

FIG. 1. (a) Results of a transcriptome-wide association study (TWAS) using univariate Cox regression analysis to correlate the expression of genes/pseudogenes with an average expression>1 counts per million (CPM) with survival, in a cohort 630 DLBCL patients whose lymphoma samples were sequenced at the Duke University (North Carolina, Reddy et al. Cell. 2017)⁵. Out of 23′560 included genes, 1530 were significantly associated with survival (p adj<0.05, light grey dots). Amongst the latter, 41 were KRAB-ZnF genes (darker dots) with a skewing of these towards an increased risk of death. (b) KRAB-ZFP genes carry a significantly higher proportion of adverse prognosis-associated genes compared to other TFs and protein coding genes.

FIG. 2. (a) 38 out of the 39 KRAB-ZnF genes associated with poor prognosis were upregulated in ABC DLBCL aggressive disease subtype. Although upregulated in ABC DLBCL, these KRAB ZnF genes conserved their significancy after including cell of origin as a covariate in our Cox regression analysis, suggesting that they are not solely a surrogate feature of the intrinsic aggressivity of the ABC subtype. (b) 32 (82%) of these KRAB-ZnF genes were located on chromosome 19, suggesting a non-random distribution, as only 56% of the members of KRAB-ZnF gene family are present on this chromosome. Most highly expressed poor prognosis KRAB-ZnF genes were enriched in the subtelomeric region of the long arm of chromosome 19 (Chr19q), a region reported as recurrently amplified in ABC disease subtype (Lenz et al., 2008)⁶ and associated to an increased risk of relapse (Dubois et al., 2019)⁷. A 200 kb region defining a hub of closely distributed poor prognosis-associated KRAB ZnF genes embedding the most highly expressed ones (ZNF586, ZNF587, ZNF587B) together with the respective paralogs (ZNF417, ZNF814) of the latter were selected for further in vitro studies.

FIG. 3. (a) The downregulation of ZNF586 in U2932 ABC and OCI-Ly7 GCB cell lines using two different shRNAs has a clear negative effect on cell growth. (b) The tandem downregulation of the ZNF587 and ZNF417 paralog pair also decreases lymphoma cell proliferation. The importance of this negative effect doesn't seem to be related to the origin of lymphoma cells, suggesting that its targeting may also be active in GCB DLBCL. (c) After 2 days of puromycin selection in the case of OCI-Ly7 (total of 4 days of knock down) and 3 days in the case of U2932 (total of 5 days of knock down) transduced cells were seeded for another 5-6 days of proliferation assessment. These assays showed a durable and profound arrest of cell proliferation.

FIG. 4. Apoptotic studies on the U2932 cell line revealed that, in parallel to cell proliferation arrest, knocked down cells demonstrated an increase in cell death in comparison to control cells (FIGS. 4A & B). Annexin V/propidium iodide (PI) staining assessed by flux cytometry revealed that this the burden of this cell death, which apparently of the necrotic form, started to surge after 4 days of gene silencing (FIG. 4 C).

FIG. 5. (a) Gene set expression analysis (GSEA) of the genes upregulated in arrested cells surviving to ZNF587/ZNF417 conjugated knock down compared to control cells revealed enrichments in pathways implicated in proteolysis, endoplasmic reticulum-associated protein degradation (ERAD), antigen processing, Major histocompatibility complex (MCH) class I presentation and cytokine production, suggestive of an increased immunogenicity of these cells. (b) Volcano and bar-/plots showing a significant upregulation of MHC class I A, B, C, E, F, G subunits, together with their common beta2-microglobulin (B2M) subunit in knocked down U2932 lymphoma cells compared to controls. In addition, CD80, a surface transmembrane protein activator of cytotoxic CD8 T cells is also upregulated in this context. (c) In human DLBCL samples (n=633), MHC class I components expression tend to be negatively correlated to ZNF587/417 gene expression, suggesting that ZNF587 and ZNF417 may contribute to immune escape of lymphoma cells.

FIG. 6. (a) The transcriptome of U2932 (and OCI-Ly7, data not shown) KD cells revealed features of partial cell differentiation, such as an upregulation of immunoglobulin heavy chains and of the master regulator of plasma cell differentiation PRDM1 (BLIMP-1), suggesting that ZNF587/417 may contribute to differentiation blockade of lymphoma cells. Looking at curated tumor suppressor gene lists (Zhao M et al. NAR, 2016)⁸, we found that the Cyclin Dependent Kinase Inhibitor 1A (CDKN1A, also known as p21) was upregulated in both lymphoma cell lines, thus possibly contributing to proliferation arrest of lymphoma cells by inhibiting the progression of lymphoma cells through the G1 to S phases of the cell cycle. (b) Western blotting of phosphorylated gH2AX showing its increase upon Z9NF587/417 conjugated KD. This suggests that their silencing generates replicative stress, thus contributing to cell cycle arrest and cell death phenotypes.

FIG. 7. (a) The negative effect of ZNF586 KD is not restricted to lymphoma cell lines. When tested on HL60 and HEK293T cell lines, these were also impacted. However, the HT29 colon cancer cell line was spared from this effect. (b) In the same way, the negative effect of ZNF587/417 conjugated KD impacted also HL60 and HEK293T cells, but in this case, the negative effect was present in all 3 colon cancer cell lines tested (i.e. Lovo, HT29 and HCT116) cell lines. Interestingly, the K562 CML cell line was not impacted by the silencing of none of these KRAB-ZnF genes, suggesting that this cell line possesses intrinsic resistance mechanisms that will deserve further exploration.

FIG. 8. In order to identify oncogenic KRAB-ZnF gene candidates, we conducted a differential gene expression analysis comparing the expression of KRAB-ZnF genes in tumors and corresponding healthy tissues from 10 TCGA tumor types. We identified 72 KRAB-ZnF genes as overexpressed in at least one of the ten studied tumor types and never downregulated, thus enriching for recurrently dysregulated KRAB-ZnF genes displaying presumable oncogenic functions. We also sought to identify KRAB-ZnF genes associated with survival by conducting a transcriptome-wide Cox univariate regression analysis, as previously described, across TCGA tumor types and Duke University dataset. We identified 52 KRAB-ZnF genes associated to poor prognosis in at least 2 tumor types. 5 KRAB-ZnF genes were associated to poor prognosis in the DLBCL dataset only. 10 KRAB-ZnF genes overlapped these features and constituted the most promising oncogenic genes candidates of our screen.

FIG. 9 In order to assess if ZNF587/417 depletion triggered a DNA damage response in different cellular contexts, we assessed gH2AX phosphorylation by flow cytometry in U2932, HBL-1 and SUDHL-4 cells. We also sought to assess if this DNA damage response was cell cycle dependent using DAPI staining. A significant increase in the number of gHA2X positive cells was seen in all of the tested cell lines. Interestingly, the cell cycle independent accumulation of gH2AX positive cells seemed to mimic exogenously induced DNA damage. a) Barplots showing the increase of gHAX positive cells upon 7.NF587/417 knock down. These FACS assessments were done 4 days after the lentiviral transduction (LV) of an shRNA targeting ZNF587 and ZNF417 simultaneously. b) Representative scatter plots of flow cytometry analyses for each assessed cell line.

FIG. 10. RNA-seq analysis of U2932 cells conducted 3 days after ZNF587/417 downregulation revealed a transcriptional signature suggestive of a UV irradiation-like DNA damage response in line with above-mentioned cell cycle-independent gH2AX accumulation. (a) Scatter plot showing the differentially expressed genes 3 days after ZNF587/417 KD. The genes belonging to the UV response UP Hallmark gene set are specified in boxes. (b) Gene set enrichment analysis (GSEA) showing UV response UP genes as the most enriched Hallmark in U2932 cells 3 days after ZNF587/417 KD.

FIG. 11. U2932 cells 3 days after ZNF587/417 KD presented a significant increase in cells with centrosome amplification suggesting the occurrence of chromosomal instability (CIN), aneuploidy and chromosomal missegregation events. a) Alpha-tubulin (spindle assembly) and DAPI (DNA content) stained U2932 cells assessed by immunofluorescence. b) Quantification of U2932 KD cells exhibiting multipolar spindles compared to controls.

FIG. 12. The DNA induced by ZNF587/417 downregulation triggers cell-type dependent cell cycle arrest. The distribution of cell cycle (CC) was performed with propidium iodide (PI) staining. DNA damage induced by ZNF587/417 downregulation triggers cell cycle arrest at different stages of the CC with U2932 cells presenting G2/M accumulation while HBL-1 and SUDHL-4 cells accumulate in S and G1 phase, respectively. This suggests that the induced DNA damage is unlikely the result of a defect in the cell cycle-dependent DNA repair machinery.

FIG. 13. U2932 KD cells present an increased susceptibility toward dendritic cell (DC) phagocyric killing. Co-culture assays were performed with human monocyte-derived dendritic cells (DCs) stained with DeepRed CellTracker® and U2932 KD cells stained with Green CMFDA. Flow cytometry analysis showed an increase of lymphoma cells being phagocytosed by DCs (double positive cell population). These co-culture assays lasted 4 hours and were conducted with KD and shScramble-transduced U2932 cells 3 days after LV transduction.

FIG. 14. ZNF587/417 KD cells present an increased immunogenicity through an upregulation of MHC class I exposure at the cell surface, suggesting that the antigenicity of these cells is presumably enhanced and that they are more proned to be engaged by cytotoxic CD8 T cells. Flow cytometry studies showing the upregulation of MHC class I on the surface of ZNF587/417-depleted cells 10 days after LV.

FIG. 15. In order to assess if this MHC class I upregulation was associated with an effective antigen processing and possible neo-antigens presentation, we decided to study the immunopeptidome of ZNF587/417-depleted cells using by immuno-affinity purification of MHC class I peptides followed by liquid chromatography-mass spectrometry (LC-MS/MS). This study showed the enrichment of MHC I bound peptides derived from proteins involved in immunes processes (dark) but also an overall increase of both MHC class I and II complexes in ZNF587/417-depleted cells compared to shScramble-transduced controls, suggesting an increase in repertoire of antigens exposed to the adaptive immune system and a reprogramming of the immunopeptidome.

FIG. 16. In order to assess the possible toxicity of ZNF587/417 downregulation in healthy human cells, we transduced primary human fibroblasts. Cell proliferation assay performed with MTT showed the absence of deleterious effects on the proliferation of human primary fibroblasts, suggesting that the genotoxic effect of the KD is seemingly restricted to transformed cells with intrinsic higher levels of CIN. The absence of CDKN1A (p21) upregulation at the mRNA levels suggests the absence of DNA damage response.

FIG. 17. Based on the transcriptomic changes seen in ZNF587/427 KD cells, we searched for potential drugs able to exhibit lethal synergistic effect on the latter. Drug sensitivity assays performed with MTT on U2932 KD cells showed cytotoxic synergism between ZNF587/417 depletion and targeted therapies such as ibrutinib (a Bruton tyrosine kinase inhibitor) and dasatinib (a PI3K/AKT inhibitor) but also the hypomethylating agent decitabine, indicating that blockade of specific compensatory oncogenic pathways and genome-wide DNA demethylation are potential therapeutic adjuncts of ZNF587/417 depletion.

### DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise/comprising” is generally used in the sense of include/including, that is to say permitting the presence of one or more features or components. The terms “comprise(s)” and “comprising” also encompass the more restricted ones “consist(s)”, “consisting” as well as “consist/consisting essentially of”, respectively.

As used in the specification and claims, the singular form “a”, “an” and “the” include plural references unless the context clearly dictates otherwise.

As used herein, “⁸ least one” means “one or more”, “two or more”, “three or more”, etc.

As used herein the terms “subject”/“subject in need thereof”, or “patient”/“patient in need thereof” are well-recognized in the art, and, are used interchangeably herein to refer to a mammal, including dog, cat, rat, mouse, monkey, cow, horse, goat, sheep, pig, camel, and, most preferably, a human. In some cases, the subject is a subject in need of treatment or a subject with a disease or disorder. However, in other aspects, the subject can be a normal subject. The term does not denote a particular age or sex. Thus, adult and newborn subjects, whether male or female, are intended to be covered. Preferably, the subject is a human, most preferably a human suffering from cancer and/or cancer metastasis or a human that might be at risk of suffering from cancer and/or cancer metastasis.

The terms “nucleic acid”, “polynucleotide,” and “oligonucleotide” are used interchangeably and refer to any kind of deoxyribonucleotide (e.g. DNA, cDNA, . . . ) or ribonucleotide (e.g. RNA, mRNA, . . . ) polymer or a combination of deoxyribonucleotide and ribonucleotide (e.g. DNA/RNA) polymer, in linear or circular conformation, and in either single- or double-stranded form. These terms are not to be construed as limiting with respect to the length of a polymer and can encompass known analogues of natural nucleotides, as well as nucleotides that are modified in the base, sugar and/or phosphate moieties (e.g. phosphorothioate backbones). In general, an analogue of a particular nucleotide has the same base-pairing specificity, i.e., an analogue of A will base-pair with T.

The term “vector”, as used herein, refers to a viral vector or to a nucleic acid (DNA or RNA) molecule such as a plasmid or other vehicle, which contains one or more heterologous nucleic acid sequence(s) of the invention and, preferably, is designed for transfer between different host cells. The terms “expression vector”, “gene delivery vector” and “gene therapy vector” refer to any vector that is effective to incorporate and express one or more nucleic acid(s) of the invention, in a cell, preferably under the regulation of a promoter. A cloning or expression vector may comprise additional elements, for example, regulatory and/or post-transcriptional regulatory elements in addition to a promoter.

The term “about,” particularly in reference to a given quantity, number or percentage, is meant to encompass deviations of plus or minus ten percent (+10). For example, about 5% encompasses any value between 4.5% to 5.5%, such as 4.5, 4.6, 4.7, 4.8, 4.9, 5, 4.1, 5.2, 5.3, 5.4, or 5.5.

According to the present invention, the cancer is a solid or a non-solid cancer. The cancer will be selected from the non-limiting group comprising carcinoma, sarcoma, melanoma, lymphoma, and leukemia. Preferably, the cancer is selected from the group comprising Acute Lymphoblastic Leukemia (ALL), Acute Myeloid Leukemia (AML), Adrenocortical Carcinoma, Adult, Childhood Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Astrocytomas, Childhood (Brain Cancer), Atypical Teratoid/Rhabdoid Tumor, Childhood, Central Nervous System (Brain Cancer), Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Childhood Breast Cancer, Childhood Bronchial Tumors, Burkitt Lymphoma, Carcinoid Tumor (Gastrointestinal), Childhood Carcinoid Tumors, Carcinoma of Unknown Primary, Cardiac (Heart) Tumors, Central Nervous System, Childhood Atypical Teratoid/Rhabdoid Tumor, Childhood Embryonal Tumors, Childhood Germ Cell Tumor, Primary CNS Lymphoma, Cervical Cancer, Childhood Cervical Cancer, Cholangiocarcinoma, Chordoma, Childhood, Chronic Lymphocytic Leukemia (CLL), Chronic Myelogenous Leukemia (CML), Chronic Myeloproliferative Neoplasms, Colorectal Cancer, Childhood Colorectal Cancer, Childhood Craniopharyngioma, Cutaneous T-Cell Lymphoma, Ductal Carcinoma In Situ (DCIS), Embryonal Tumors, Endometrial Cancer (Uterine Cancer), Childhood Ependymoma, Esophageal Cancer, Childhood Esophageal Cancer, Esthesio neuroblastoma, Ewing Sarcoma (Bone Cancer), Childhood Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone (Malignant, and Osteosarcoma), Gallbladder Cancer, Gastric, (Stomach) Cancer, Childhood Gastric (Stomach) Cancer, Gastrointestinal Carcinoid Tumor, Gastrointestinal Stromal Tumors (GIST) (Soft Tissue Sarcoma), Childhood Gastrointestinal Stromal Tumors, Germ Cell Tumors, Childhood Central Nervous System Germ Cell Tumors (Brain Cancer), Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Childhood Head and Neck Cancers, Childhood Heart Tumors, Hepatocellular (Liver) Cancer, Langerhans Cell Histiocytosis, Hodgkin Lymphoma, Hypopharyngeal Cancer (Head and Neck Cancer), Intraocular Melanoma, Childhood Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Childhood Laryngeal Cancer, Papillomatosis, Leukemia, Lip and Oral Cavity Cancer (Head and Neck Cancer), Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Childhood Melanoma, Intraocular (Eye)Melanoma, Childhood Intraocular Melanoma, Merkel Cell Carcinoma (Skin Cancer), Malignant Mesothelioma, Childhood Mesothelioma, Metastatic Squamous Neck Cancer with Occult Primary (Head and Neck Cancer), Midline Tract Carcinoma Involving NUT Gene, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplasia Syndromes, Myelodysplastic/Myeloproliferative Neoplasms, Myelogenous Leukemia, Chronic (CML), Myeloid Leukemia, Acute (AML), Chronic Myeloproliferative Neoplasms, Nasal Cavity and Paranasal Sinus Cancer (Head and Neck Cancer), Nasopharyngeal Cancer (Head and Neck Cancer), Childhood Nasopharyngeal Cancer, Neuroblastoma, Non-Hodgkin Lymphoma (NHL) such as diffuse large B cell lymphoma, Non-Small Cell Lung Cancer, Oral Cancer, Lip and Oral Cavity Cancer and Oropharyngeal Cancer (Head and Neck Cancer), Childhood Oral Cavity Cancer, Osteosarcoma and Malignant Fibrous Histiocytoma of Bone, Ovarian Cancer, Childhood Ovarian Cancer, Pancreatic Cancer, Childhood Pancreatic Cancer, Pancreatic Neuroendocrine Tumors (Islet Cell Tumors), Papillomatosis, Paraganglioma, Childhood Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Childhood Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Childhood Rhabdomyosarcoma (Soft Tissue Sarcoma), Salivary Gland Cancer (Head and Neck Cancer), Childhood Salivary Gland Tumors, Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Uterine Sarcoma, Sezary Syndrome (Lymphoma), Skin Cancer, Childhood Skin Cancer, Small Cell Lung Cancer, Small Intestine Cancer, Soft Tissue Sarcoma, Squamous Cell Carcinoma of the Skin, Squamous Neck Cancer with Occult Primary, Metastatic (Head and Neck Cancer), Stomach (Gastric) Cancer, Childhood Stomach (Gastric) Cancer, T-Cell Lymphoma, Cutaneous, Testicular Cancer, Childhood Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Childhood Thyroid Tumors, Transitional Cell Cancer of the Renal Pelvis and Ureter (Kidney (Renal Cell) Cancer), Carcinoma of Unknown Primary, Childhood Cancer of Unknown Primary, Unusual Cancers of Childhood, Ureter and Renal Pelvis, Transitional Cell Cancer (Kidney (Renal Cell) Cancer, Urethral Cancer, Endometrial Uterine Cancer, Uterine Sarcoma, Vaginal Cancer, Childhood Vaginal Cancer, Vascular Tumors (Soft Tissue Sarcoma), Vulvar Cancer, and Wilms Tumor or a combination of one of more of these cancers

In some aspects, the cancer is a non-solid/hematological cancer, most preferably, the cancer is aggressive non-Hodgkin lymphoma.

The terms “KRAB-containing zinc finger proteins”, “KZFP”, “KRAB-ZnF”, and “KRAB-ZFP” are used interchangeably herein to refer to KRAB-containing zinc finger proteins (KZFPs) that constitute a large family of transcription factors notably involved in controlling the expression of transposable element-embedded regulator sequences (TEeRS)^{9,10}. KZFPs and their genomic targets contribute to regulating many biological events, from early embryogenesis to brain development, and from immune responses to liver metabolism^{11, 12}.

While focusing on the role of these KZFP, the Inventors surprisingly discovered the role of several KZFP, the expression of which, individually or in combination, were associated with cancer and, for some of them with poor cancer outcome. The genes encoding said one or more KZFP are, generally, clustering on one of the following human chromosomes: 1, 7, 8, 16, 17, 19 or 20.

The present invention thus provides an agent modulating the expression and/or activity of one or more i) KRAB-containing zinc finger protein (KZFP), for use in the treatment and/or prevention of cancer and/or cancer metastasis in a subject in need thereof.

Preferably, the agent of the invention modulates the activity of the one or more KZFP protein by enhancing and/or favoring the degradation of said one or more KZFP (Sievers et al., 2018).

In one aspect, said one or more KZFP is selected from the group comprising those listed in Table 1, or a combination of one or more thereof.

TABLE 1

| | | | | | cDNA | Protein |
|---|---|---|---|---|---|---|
| KZFP | Chr. | Start | End | NCBI ref | SEQ ID No. | SEQ ID No. |
| ZNF671 | 19 | 58231118 | 58238992 | NM_024833.3 | 1 | 2 |
| ZNF776 | 19 | 58258182 | 58269516 | NM_173632.4 | 3 | 4 |
| ZNF586 | 19 | 58281043 | 58291984 | NM_017652.4 | 5 | 6 |
| ZNF552 | 19 | 58318449 | 58326281 | NM_024762.3 | 7 | 8 |
| ZNF587B | 19 | 58341666 | 58357606 | NM_001376223.1 | 9 | 10 |
| ZNF587 | 19 | 58361226 | 58376485 | NM_032828.4 | 11 | 12 |
| ZNF814 | 19 | 58380748 | 58400405 | NM_001144989.2 | 13 | 14 |
| ZNF417 | 19 | 58417141 | 58427960 | NM_152475.3 | 15 | 16 |
| PRDM9 | 5 | 23507717 | 23528202 | NM_020227.4 | 43 | 44 |
| RBAK | 7 | 5085490 | 5109118 | NM_021163.4 | 45 | 46 |
| ZFP30 | 19 | 38121906 | 38146366 | NM_001320669.3 | 47 | 48 |
| ZFP69B | 1 | 40916147 | 40929387 | NM_023070.3 | 49 | 50 |
| ZFP82 | 19 | 36879531 | 36909546 | NM_133466.4 | 51 | 52 |
| ZKSCAN2 | 16 | 25247321 | 25269166 | NM_001012981.5 | 53 | 54 |
| ZKSCAN4 | 6 | 28209474 | 28220047 | NM_019110.5 | 55 | 56 |
| ZNF101 | 19 | 19779647 | 19794315 | NM_033204.4 | 57 | 58 |
| ZNF107 | 7 | 64126510 | 64171955 | NM_001282359.2 | 59 | 60 |
| ZNF117 | 7 | 64432153 | 64451414 | NM_015852.4 | 61 | 62 |
| ZNF121 | 19 | 9671004 | 9695180 | NM_001008727.5 | 63 | 64 |
| ZNF124 | 1 | 247318290 | 247335322 | NM_001297568.2 | 65 | 66 |
| ZNF134 | 19 | 58125609 | 58136092 | NM_003435.5 | 67 | 68 |
| ZNF140 | 12 | 133657508 | 133684014 | NM_003440.4 | 69 | 70 |
| ZNF160 | 19 | 53569866 | 53606675 | NM_001322131.2 | 71 | 72 |
| ZNF17 | 19 | 57922528 | 57933302 | NM_001330617.2 | 73 | 74 |
| ZNF18 | 17 | 11880755 | 11900792 | NM_001303281.2 | 75 | 76 |
| ZNF184 | 6 | 27418525 | 27440884 | NM_001318891.2 | 77 | 78 |
| ZNF195 | 11 | 3379156 | 3400376 | NM_001130520.3 | 79 | 80 |
| ZNF200 | 16 | 3272324 | 3285158 | NM_198088.3 | 81 | 82 |
| ZNF202 | 11 | 123594621 | 123612368 | NM_003455.4 | 83 | 84 |
| ZNF205 | 16 | 3162586 | 3170518 | NM_001042428.2 | 85 | 86 |
| ZNF212 | 7 | 148936741 | 148952697 | NM_012256.4 | 87 | 88 |
| ZNF222 | 19 | 44529530 | 44537263 | NM_001129996.2 | 89 | 90 |
| ZNF226 | 19 | 44669252 | 44681838 | NM_001032373.2 | 91 | 92 |
| ZNF239 | 10 | 44051791 | 44070064 | NM_001099282.2 | 93 | 94 |
| ZNF251 | 8 | 145946293 | 145980916 | NM_138367.2 | 95 | 96 |
| ZNF26 | 12 | 133562952 | 133603688 | NM_019591.4 | 97 | 98 |
| ZNF263 | 16 | 3333490 | 3341460 | NM_005741.5 | 99 | 100 |
| ZNF267 | 16 | 31885127 | 31928678 | NM_003414.6 | 101 | 102 |
| ZNF282 | 7 | 148892637 | 148923329 | NM_003575.4 | 103 | 104 |
| ZNF283 | 19 | 44331472 | 44356169 | NM_181845.2 | 105 | 106 |
| ZNF284 | 19 | 44576311 | 44593766 | NM_001037813.4 | 107 | 108 |
| ZNF3 | 7 | 99667595 | 99679346 | NM_032924.5 | 109 | 110 |
| ZNF317 | 19 | 9251072 | 9274089 | NM_020933.5 | 111 | 112 |
| ZNF320 | 19 | 53379432 | 53400888 | NM_001351774.2 | 113 | 114 |
| ZNF324B | 19 | 58962988 | 58969200 | NM_207395.3 | 115 | 116 |
| ZNF337 | 20 | 25653830 | 25677489 | NM_015655.4 | 117 | 118 |
| ZNF343 | 20 | 2462462 | 2489561 | NM_024325.6 | 119 | 120 |
| ZNF354A | 5 | 178138512 | 178157660 | NM_005649.3 | 121 | 122 |
| ZNF419 | 19 | 57999117 | 58007466 | NM_024691.4 | 123 | 124 |
| ZNF420 | 19 | 37569334 | 37621270 | NM_144689.5 | 125 | 126 |
| ZNF432 | 19 | 52534630 | 52552091 | NM_014650.4 | 127 | 128 |
| ZNF473 | 19 | 50529261 | 50552033 | NM_015428.4 | 129 | 130 |
| ZNF479 | 7 | 57185382 | 57200143 | NM_001370129.2 | 131 | 132 |
| ZNF480 | 19 | 52800421 | 52829175 | NM_144684.4 | 133 | 134 |
| ZNF485 | 10 | 44101885 | 44113352 | NM_145312.4 | 135 | 136 |
| ZNF496 | 1 | 247460716 | 247495169 | NM_032752.3 | 137 | 138 |
| ZNF500 | 16 | 4798239 | 4817163 | NM_021646.4 | 139 | 140 |
| ZNF514 | 2 | 95810744 | 95825593 | NM_032788.3 | 141 | 142 |
| ZNF517 | 8 | 146024297 | 146035469 | NM_213605.3 | 143 | 144 |
| ZNF525 | 19 | 53868957 | 53889843 | NM_001348156.2 | 145 | 146 |
| ZNF529 | 19 | 37034514 | 37064194 | NM_020951.5 | 147 | 148 |
| ZNF530 | 19 | 58111252 | 58121466 | NM_001321981.2 | 149 | 150 |
| ZNF543 | 19 | 57831839 | 57842138 | NM_213598.4 | 151 | 152 |
| ZNF544 | 19 | 58740279 | 58775010 | NM_014480.4 | 153 | 154 |
| ZNF547 | 19 | 57874918 | 57890933 | NM_173631.4 | 155 | 156 |
| ZNF550 | 19 | 58053205 | 58071231 | NM_001397372.1 | 157 | 158 |
| ZNF558 | 19 | 8916845 | 8942980 | NM_144693.3 | 159 | 160 |
| ZNF565 | 19 | 36673177 | 36705567 | NM_152477.5 | 161 | 162 |
| ZNF567 | 19 | 37178513 | 37212238 | NM_001322917.1 | 163 | 164 |
| ZNF577 | 19 | 52370300 | 52391204 | NM_001370449.1 | 165 | 166 |
| ZNF583 | 19 | 56915700 | 56938733 | NM_152478.3 | 167 | 168 |
| ZNF584 | 19 | 58920015 | 58929694 | NM_173548.3 | 169 | 170 |
| ZNF600 | 9 | 53267436 | 53290044 | NM_001321866.4 | 171 | 172 |
| ZNF614 | 19 | 52516576 | 52531632 | NM_025040.4 | 173 | 174 |
| ZNF615 | 19 | 52494584 | 52511464 | NM_001199324.2 | 175 | 176 |
| ZNF679 | 7 | 63688851 | 63727309 | NM_153363.3 | 177 | 178 |
| ZNF695 | 1 | 247148624 | 247171359 | NM_020394.5 | 179 | 180 |
| ZNF7 | 8 | 146052944 | 146069157 | NM_003416.4 | 181 | 182 |
| ZNF700 | 19 | 12035921 | 12061578 | NM_144566.3 | 183 | 184 |
| ZNF705B | 8 | 7783858 | 7809935 | NM_001193630.1 | 185 | 186 |
| ZNF707 | 8 | 144766648 | 144777555 | NM_001100598.2 | 187 | 188 |
| ZNF71 | 19 | 57106669 | 57135854 | NM_001370215.1 | 189 | 190 |
| ZNF713 | 7 | 55955148 | 56009918 | NM_182633.3 | 191 | 192 |
| ZNF716 | 7 | 57509882 | 57533265 | NM_001159279.1 | 193 | 194 |
| ZNF724 | 19 | 23404400 | 23433196 | NM_001355404.2 | 195 | 196 |
| ZNF736 | 7 | 63774338 | 63817012 | NM_001170905.3 | 197 | 198 |
| ZNF74 | 22 | 20748440 | 20762745 | NM_003426.4 | 199 | 200 |
| ZNF746 | 7 | 149169884 | 149194900 | NM_001394198.1 | 201 | 202 |
| ZNF749 | 19 | 57946692 | 57958469 | NM_001023561.4 | 203 | 204 |
| ZNF761 | 19 | 53935236 | 53961515 | NM_001289951.2 | 205 | 206 |
| ZNF765 | 19 | 53898401 | 53915262 | NM_001040185.3 | 207 | 208 |
| ZNF766 | 19 | 52772839 | 52799299 | NM_001010851.3 | 209 | 210 |
| ZNF773 | 19 | 58011305 | 58019597 | NM_198542.3 | 211 | 212 |
| ZNF782 | 9 | 99578550 | 99616796 | NM_001001662.3 | 213 | 214 |
| ZNF783 | 7 | 148959286 | 148982079 | NM_001195220.2 | 215 | 216 |
| ZNF785 | 16 | 30591976 | 30597018 | NM_152458.7 | 217 | 218 |
| ZNF789 | 7 | 99070512 | 99085228 | NM_213603.3 | 219 | 220 |
| ZNF80 | 3 | 113953477 | 113956425 | NM_007136.4 | 221 | 222 |
| ZNF805 | 19 | 57751999 | 57774096 | NM_001023563.4 | 223 | 224 |
| ZNF83 | 19 | 53115629 | 53193749 | NM_001277945.2 | 225 | 226 |
| ZNF84 | 12 | 133614094 | 133639885 | NM_001289971.2 | 227 | 228 |
| ZNF841 | 9 | 52567717 | 52599018 | NM_001136499.2 | 229 | 230 |
| ZNF850 | 19 | 37234381 | 37263709 | NM_001193552.2 | 231 | 232 |
| ZNF93 | 19 | 20011754 | 20046384 | NM_031218.4 | 233 | 234 |
| ZSCAN22 | 19 | 58838359 | 58853698 | NM_181846.3 | 235 | 236 |
| ZSCAN29 | 15 | 43650372 | 43663241 | NM_001372080.1 | 237 | 238 |
| ZNF252P | 8 | 146198975 | 146228298 | ENST00000528327 | 239 | N/A |
| ENSG00000269825 | 19 | 53153700 | 53193786 | ENST00000598322 | 240 | 241 |
| ZNF8-ERVK3-1 | 19 | 58790332 | 58826563 | ENST00000591325 | 242 | N/A |

Table 1 contains a list of 110 KZFP genes with pro-oncogenic characteristics encompassing upregulated and never downregulated KZFPs in at least one of ten broad TCGA cancer categories and KZFPs associated with patient survival in at least two TCGA cancer categories and/or in the Duke's University DLBCL dataset

In one aspect, the combination of one or more KZFP will comprise a combination of two or more, three or more, four or more, five or more, six or more, seven or more, ten or more, fifteen or more, twenty or more, twenty-five or more, thirty or more, forty or more, fifty or more, sixty or more, seventy or more, eighty or more, ninety or more, one hundred or more, or the complete set of the 110 KZFPs.

In one aspect, the KZFP will be selected from the group comprising ZNF671, ZNF776, ZNF586, ZNF552, ZNF587B, ZNF417, ZNF107, ZNF121, ZNF200, ZNF239, ZNF473, ZNF547, ZNF558, ZNF587, ZNF695, ZNF7, ZNF700, ZNF749, ZNF766, ZNF783, ZNF789, ZNF814, ZNF841, ZNF8-ERVK3-1 and ZNF850, or a combination of one or more thereof, e.g. two or more, three or more, four or more, five or more, six or more, seven or more, ten or more, fifteen or more, twenty or more, or the complete set of the 25 KZFPs.

In a preferred aspect, the KZFP will be selected from the group comprising ZNF107, ZNF121, ZNF200, ZNF239, ZNF473, ZNF547, ZNF558, ZNF587, ZNF695, ZNF7, ZNF700, ZNF749, ZNF766, ZNF783, ZNF789, ZNF814, ZNF841, ZNF8-ERVK3-1 and ZNF850, or a combination of one or more thereof, e.g. two or more, three or more, four or more, five or more, six or more, seven or more, ten or more, fifteen or more, or the complete set of the 19 KZFPs.

In a more preferred aspect, the KZFP will be selected from the group comprising ZNF121, ZNF200, ZNF473, ZNF547, ZNF587, ZNF7, ZNF749, ZNF766, ZNF814, and ZNF850, or a combination of one or more thereof, e.g. two or more, three or more, four or more, five or more, six or more, seven or more, nine or more, or the complete set of the 10 KZFPs.

In one aspect, the KZFP will be selected from the group comprising SEQ ID No. 12, SEQ ID No. 14, SEQ ID No. 64, SEQ ID No. 82, SEQ ID No. 130, SEQ ID No. 156, SEQ ID No. 182, SEQ ID No. 204, SEQ ID No. 210, and SEQ ID No. 232, a fragment or a variant thereof or a combination of one or more thereof.

In other aspects, the agent inhibits and/or impairs the binding of the KZFP to a target genomic DNA (e.g. by targeting the DNA comprising transposable element-embedded regulator sequences (TEeRS) or the zinc-finger motif of the C2H2 zinc finger motif and/or leading to proteasomal degradation), and/or the agent inhibits or impairs the binding of KZFP to its interacting protein(s) or other molecule(s) via which it controls heterochromatin and DNA methylation (e.g. scaffold protein KAP1).

In other aspects, the agent is an antibody, or antigen binding fragment thereof, that i) inhibits and/or impairs the binding of the KZFP to a target genomic DNA (e.g. by specifically targeting the zinc-finger motif of the C2H2 zinc finger motif), and/or the binding of KZFP to its interacting protein(s) or other molecule(s).

As used herein, an “antibody” is a protein molecule that reacts with a specific antigenic determinant or epitope and belongs to one or five distinct classes based on structural properties: IgA, IgD, IgE, IgG and IgM. The antibody may be a polyclonal (e.g. a polyclonal serum) or a monoclonal antibody, including but not limited to fully assembled antibody, single chain antibody, antibody fragment, and chimeric antibody, humanized antibody as long as these molecules are still biologically active and still bind to at least one peptide or protein of the invention. Preferably the antibody is a monoclonal antibody. Preferably also the monoclonal antibody will be selected from the group comprising IgG1, IgG2, IgG2a, IgG2b, IgG3 and IgG4 or a combination thereof. Most preferably, the monoclonal antibody is selected from the group comprising IgG1, IgG2, IgG2a, and IgG2b, or a combination thereof.

An “antigen binding fragment” comprises a portion of a full-length antibody. Examples of antigen binding fragments include Fab, Fab′, F(ab′)2, and Fv fragments; diabodies; linear antibodies; single-chain antibody molecules; and multispecific antibodies formed from antibody fragments.

The present invention also provides an agent modulating the expression and/or activity of one or more transcript, preferably one or more mRNA, encoding a KZFP of the invention, for use in the treatment and/or prevention of cancer and/or cancer metastasis in a subject in need thereof. Preferably, the agent of the invention modulates the expression and/or activity by inhibiting the translation of said one or more mRNA encoding a KZFP.

As used herein, a messenger RNA (mRNA) refers to a single-stranded RNA molecule that is complementary to one of the DNA strands of a gene, e.g. a gene encoding a KZFP. The mRNA is an RNA version of the gene that leaves the cell nucleus and moves to the cytoplasm where proteins are made.

Since a single gene often has more than one transcript, one or ordinary skill in the art will appreciate that the term mRNA also encompasses fragments and variants of an mRNA of the invention. Non-limiting examples of mRNA variants comprise precursor mRNA and splicing variants. Alternative splicing is a widely used mechanism for the formation of isoforms or splicing variants. In this process, which occurs during gene expression, the exons of a gene may be included or excluded in the processed mRNA.

Preferably, the at least one transcript encoding a KZFP of the invention is selected from the group comprising, represented by, or corresponding to, cDNA sequences: SEQ ID No. 11, SEQ ID No. 13, SEQ ID No. 63, SEQ ID No. 81, SEQ ID No. 129, SEQ ID No. 155, SEQ ID No. 181, SEQ ID No. 203, SEQ ID No. 209, and SEQ ID No. 231, a fragment or a variant thereof or a combination of one or more thereof.

As used herein, the term “variant” refers to biologically active derivatives of a nucleic acid or peptide sequence. In general, the term “variant” refers to molecules having a native sequence and structure with one or more additions, substitutions (generally conservative in nature) and/or deletions (e.g. alternative splicing variants), relative to the native molecule, so long as the modifications do not destroy biological activity and which are “substantially homologous” to the reference molecule. In general, the sequences of such variants are functionally, i.e. biologically, active variants and will have a high degree of sequence homology to the reference sequence, e.g., sequence homology of more than 50%, generally more than 60%-70%, even more particularly 80% or more, such as at least 90% or 95% or more, when the two sequences are aligned.

Alternatively, the term “variant” also refers to post-transcriptionally modified nucleic acid sequences of the invention, i.e. methylation, phosphorylation, etc. . . .

As used herein, a “fragment” of one or more nucleic acid sequence or polypeptide sequence of the invention refers to a sequence containing less nucleotides or amino acids in length than the respective sequences of the invention while retaining the biological activity described herein. Preferably, this fragment contains, at least 10%, 20%, 30%, 40%, 50%, 60%, 70%, 80%, or 90% of the entire length of the reference nucleic acid sequence or polypeptide sequence.

As used herein, “Homology” refers to the percent identity between two polynucleotide or two polypeptide sequences. Two nucleic acid, or two polypeptide sequences are “substantially homologous” to each other when the sequences exhibit at least about 50% sequence identity, preferably at least about 75% sequence identity, more preferably at least about 80% sequence identity, more preferably at least about 90% sequence identity, and most preferably at least about 95%-98% sequence identity over a defined length of the molecules. As used herein, substantially homologous also refers to sequences showing complete identity to the specified sequence.

In general, “identity” refers herein to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications.

Alternatively, homology can be determined by readily available computer programs or by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art.

The present invention further provides an agent modulating the expression and/or activity of one or more KZFP gene of the invention, for use in the treatment and/or prevention of cancer and/or cancer metastasis in a subject in need thereof.

Preferably, the agent of the invention modulates the expression and/or activity by inhibiting the transcription of one or more KZFP gene encoding a KZFP.

Also envisioned are combinations of one or more approaches or agents described herein for use in the treatment and/or prevention of cancer and/or cancer metastasis in a subject in need thereof.

Preferably, the agent of the invention is selected from the group comprising a nucleic acid, a chemical compound, a peptide or analog thereof, an antibody or an antigen-binding fragment thereof, and an antibody mimetic, or a combination of one or more thereof.

As used herein, a “chemical agent” is a compound that produces change by virtue of its chemical composition and its effects on living tissues and organisms. The chemical agent may be a small molecule inhibitor (SMI) and is preferably a non-peptidyl molecule inhibitor of KZFPs.

The chemical agents of the invention can be tested using a number of techniques known to those of skill in the art. For example, tagged KZFPs or cell lines transfected and expressing a KZFP coupled to a reporter gene such as Luciferase or green fluorescent protein may be used in assay screens for inhibitors of KZFPs. Computational zinc finger docking and biochemical analysis can be conducted in order to identify molecules able to bring ZNF proteins to the Cereblon complex for subsequent ubiquitination and proteasomal degradation Non-limiting examples of chemical agents modulating the expression and/or activity of one or more KZFP of the invention are selected among the non-limiting group comprising agents inducing ubiquitination and proteasomal degradation. Non-limiting examples of such agents comprise thalidomide and its analogues and derivatives (e.g. lenalidomide and pomalidomide).

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

In case the agent of the invention is a peptide, then it will preferably be conjugated to an agent that increases the accumulation of the peptide in the cancer cell. Such an agent can be a compound which induces receptor mediated endocytosis such as for example the membrane transferrin receptor mediated endocytosis of transferrin conjugated to therapeutic drugs (Qian Z. M. et al., “Targeted drug delivery via the transferrin receptor-mediated endocytosis pathway” Pharmacological Reviews, 54, 561, 2002) or a cell membrane permeable carrier which can, be selected e.g. among the group of fatty acids such as decanoic acid, myristic acid and stearic acid, which have already been used for intracellular delivery of peptide inhibitors of protein kinase C (Ioannides C. G. et al., “Inhibition of IL-2 receptor induction and IL-2 production in the human leukemic cell line Jurkat by a novel peptide inhibitor of protein kinase C” Cell Immunol., 131, 242, 1990) and protein-tyrosine phosphatase (Kole H. K. et al., “A peptide-based protein-tyrosine phosphatase inhibitor specifically enhances insulin receptor function in intact cells” J. Biol. Chem. 271, 14302, 1996) or among peptides. Preferably, cell membrane permeable carriers are used, more preferably a cell membrane permeable carrier peptide is used.

In case the cell membrane permeable carrier is a peptide then it will preferably be a positively charged amino acid rich peptide. Preferably such positively charged amino acid rich peptide is an arginine rich peptide. It has been shown in Futaki et al. (Futaki S. et al., “Arginine-rich peptides. An abundant source of membrane-permeable peptides having potential as carriers for intracellular protein delivery” J. Biol. Chem., 276, 5836, 2001), that the number of arginine residues in a cell membrane permeable carrier peptide has a significant influence on the method of internalization and that there seems to be an optimal number of arginine residues for the internalization, preferably they contain more than 6 arginines, more preferably they contain 9 arginines (R9).

The peptide may be conjugated to the cell membrane permeable carrier by a spacer (e.g. two glycine residues). In this case, the cell membrane permeable carrier is preferably a peptide. Usually, arginine rich peptides are selected from the group comprising the HIV-TAT 48-57 peptide, the FHV-coat 35-49 peptide, the HTLV-II Rex 4-16 peptide and the BMV gag 7-25 peptide. Preferably, the arginine rich peptide is HIV-TAT 48-57 peptide.

Since an inherent problem with native peptides (in L-form) is degradation by natural proteases, the peptide, as well as the cell membrane permeable peptide, of the invention may be prepared to include D-forms and/or “retro-inverso isomers” of the peptide.

In this case, retro-inverso isomers of fragments and variants of the peptide, as well as of the cell membrane permeable peptide, of the invention are prepared according to techniques known in the art.

The agent of the invention may also be a “nucleic acid”. The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

Examples of nucleic acid agents of the invention comprise those selected among the group comprising an acid nucleic encoding an miRNA, an siRNA, a piRNA, an hnRNA, an snRNA, an sg RNA, an esiRNA, an shRNA, and an antisense oligonucleotide (e.g. modified ASO), or a combination thereof.

The terms “microRNA,” “miRNA,” and “MiR” are interchangeable and refer to endogenous or artificial non-coding RNAs that are capable of regulating gene expression. It is believed that miRNAs function via RNA interference.

The terms “siRNA” and “short interfering RNA” are interchangeable and refer to single-stranded or double-stranded RNA molecules that are capable of inducing RNA interference. SiRNA molecules typically have a duplex region that is between 18 and 30 base pairs in length.

The terms “piRNA” and “Piwi-interacting RNA” are interchangeable and refer to a class of small RNAs involved in gene silencing. PiRNA molecules typically are between about 26 and about 31 nucleotides in length.

Examples of antisense oligonucleotides (ASOs) include the GapmeRs. As used herein, a GapmeR is a chimeric antisense oligonucleotide that contains a central block of deoxynucleotide monomers sufficiently long to induce RNase H cleavage. Usually, the GapmeRs of the invention are directed against one or more mRNA encoding a KZFP of the invention. Modified ASOs such as modified GapmeRs are also encompassed in the present invention and comprise, e.g. GapmeRs with fixed chemical modification architectures selected from the group comprising i) a gapmer with five 2′-O-methoxyethyl (MOE) modifications in each flank, and a central gap of 10 unmodified dans (e.g. 5-10-5 MOE design), and ii) a gapmer employing three or four locked nucleic acid (LNA) modifications in each flank (e.g. 3-10-3 or 4-8-4 LNA designs), as well as a combination of one or more thereof.

The terms “sgRNA” and “guideRNA” are interchangeable and refer to a specific RNA sequence that recognizes the target DNA region of interest and directs the endonuclease there for editing. The gRNA is usually made up of two parts: crispr RNA (crRNA), a 17-20 nucleotide sequence complementary to the target DNA, and a tracr RNA, which serves as a binding scaffold for the Cas nuclease.

Any suitable engineered sgRNA, or crRNA and tracrRNA, can be employed as long as it is effective for recognizing a target DNA of the invention. The design of such sgRNA, or crRNA and tracrRNA, is within the skill of ordinary artisans.

The terms “shRNA” as used herein refers to a nucleic acid molecule comprising at least two complementary portions hybridized or capable of specifically hybridizing to form a duplex structure sufficiently long to mediate RNAi (typically between about 15 to about 29 nucleotides in length), and at least one single-stranded portion, typically between approximately 1 and about 10 nucleotides in length that forms a loop connecting the ends of the two sequences that form the duplex. Exemplary shRNA of the invention may, e.g. be selected from the non-limiting group comprising those listed in table 2 (represented by their DNA sequence) that were designed to be uniquely and selectively recognizing a target sequence within an mRNA encoding a KZFP, or a combination of KZFP, of the invention. Selecting a suitable shRNA is well within the competences of one of ordinary skill in the art using routine experimentation, several commercial and noncommercial web sites available for shRNA design as well as the information are provided herein.

Non-limiting examples of target sequences within an mRNA encoding a KZFP, or a combination of KZFP, of the invention are also given in Table 2. One of ordinary skill in the art, using routine experimentation, several available commercial and noncommercial web sites for determining said target sequences as well as relevant information provided herein is able to determine a target sequence within an mRNA encoding a KZFP, or a combination of KZFP

TABLE 2

| KZFP | Target Sequence | Hairpin Sequence (DNA sequence) |
|---|---|---|
| ZNF586 v.1 | GCTTATACATCTAGTCTCATT | 5′-CCGG-GCTTATACATCTAGTCTCATT-CTCGAG |
| | (SEQ ID No. 17) | AATGAGACTAGATGTATAAGC-TTTTT-3′ |
| | | (SEQ ID No. 18) |
| ZNF586 v.2 | GCAGCAAATATGGGAAATTAT | 5′-CCGG-GCAGCAAATATGGGAAATTAT-CTCGAG- |
| | (SEQ ID No. 19) | ATAATTTCCCATATTTGCTGC-TTTTT-3′ |
| | | (SEQ ID No. 20) |
| ZNF671 v.1 | GCCAAAGCTATGACCTCTTTA | 5′-CCGG-GCCAAAGCTATGACCTCTTTA-CTCGAG- |
| | (SEQ ID No. 21) | TAAAGAGGTCATAGCTTTGGC-TTTTT-3′ |
| | | (SEQ ID No. 22) |
| ZNF587- | AGTCGAAAGAGCAGCCTTATT | 5′-CCGG-AGTCGAAAGAGCAGCCTTATT-CTCGAG- |
| ZNF417 v.1 | (SEQ ID No. 23) | AATAAGGCTGCTCTTTCGACT-TTTTTTGAAT-3′ |
| | | (SEQ ID No. 24) |
| ZNF587- | GCAGCATATTGGAGAGAAATT | 5′-CCGG-GCAGCATATTGGAGAGAAATT-CTCGAG- |
| ZNF417 v.2 | (SEQ ID No. 25) | AATTTCTCTCCAATATGCTGC-TTTTT-3′ |
| | | (SEQ ID No. 26) |
| ZNF671 v.2 | CGGGAAGAATGGGAGCTTCTT | 5′-CCGG-CGGGAAGAATGGGAGCTTCTT-CTCGAG- |
| | (SEQ ID No. 27) | AAGAAGCTCCCATTCTTCCCG-TTTTT-3′ |
| | | (SEQ ID No. 28) |
| ZNF776 v.1 | CGGGAGAAAGACATCATGAAT | 5′-CCGG-CGGGAGAAAGACATCATGAAT-CTCGAG- |
| | (SEQ ID No. 29) | ATTCATGATGTCTTTCTCCCG-TTTTT-3′ |
| | | (SEQ ID No. 30) |
| ZNF776 v.2 | TATGTCACATGAGCCATTTAT | 5′-CCGG-TATGTCACATGAGCCATTTAT-CTCGAG- |
| | (SEQ ID No. 31) | ATAAATGGCTCATGTGACATA-TTTTTG-3′ |
| | | (SEQ ID No. 32) |
| ZNF552 v.1 | GCAGATCATCAGGGAACACAT | 5′-CCGG-GCAGATCATCAGGGAACACAT-CTCGAG- |
| | (SEQ ID No. 33) | ATGTGTTCCCTGATGATCTGC-TTTTT-3′ |
| | | (SEQ ID No. 34) |
| ZNF552 v.2 | GCTCTACATTCCGTGTTCATA | 5′-CCGG-GCTCTACATTCCGTGTTCATA-CTCGAG- |
| | (SEQ ID No. 35) | TATGAACACGGAATGTAGAGC-TTTTT-3′ |
| | | (SEQ ID No. 36) |
| ZNF552- | CCTTCTAAGCAGAGTATTTAT | 5′-CCGG-CCTTCTAAGCAGAGTATTTAT-CTCGAG- |
| ZNF587B- | (SEQ ID No. 37) | ATAAATACTCTGCTTAGAAGG-TTTTT-3′ |
| ZNF814 v.1 | | (SEQ ID No. 38) |
| ZNF552- | CAGCACCAGAATGAGCACATT | 5′-CCGG-CAGCACCAGAATGAGCACATT-CTCGAG- |
| ZNF587B- | (SEQ ID No. 39) | AATGTGCTCATTCTGGTGCTG-TTTTT-3′ |
| ZNF814 v.2 | | (SEQ ID No. 40) |
| ZNF552- | GCAGATCATCAGGGAACACAT | 5′-CCGG-GCAGATCATCAGGGAACACAT-CTCGAG- |
| ZNF587B- | (SEQ ID No. 41) | ATGTGTTCCCTGATGATCTGC-TTTTT-3′ |
| ZNF814 v.3 | | (SEQ ID No. 42) |

It is also understood that the above target sequences can be recognized and targeted also by any miRNA, siRNA, piRNA, hnRNA, snRNA, sg RNA, esiRNA, and antisense oligonucleotide (e.g. modified ASO) of the invention.

The terms “snRNA” and “small nuclear RNA” are interchangeable and refer to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs.

In particular, the invention therefore provides isolated siRNA comprising short double-stranded RNA from about 18 to about 30 nucleotides in length, that are targeted to the mRNA encoding a KZFP of the invention.

The term “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered. The siRNAs of the invention can comprise partially purified RNA, substantially pure RNA, synthetic RNA, or recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion.

One or both strands of the siRNA of the invention can also comprise a 3′ overhang. A “3′ overhang” refers to at least one unpaired nucleotide extending from the 3′-end of an RNA strand. Thus, in one aspect, the siRNA of the invention comprises at least one 3′ overhang of from one to about six nucleotides (which includes ribonucleotides or deoxynucleotides) in length, preferably from one to about five nucleotides in length, more preferably from one to about four nucleotides in length, and particularly preferably from about one to about two nucleotides in length.

In the case both strands of the siRNA molecule comprise a 3′ overhang, the length of the overhangs can be the same or different for each strand. In a most preferred embodiment, the 3′ overhang is present on both strands of the siRNA, and is two nucleotides in length. In order to enhance the stability of the present siRNAs, the 3′ overhangs can also be stabilized against degradation. In one embodiment, the overhangs are stabilized by including purine nucleotides, such as adenosine or guanosine nucleotides.

Alternatively, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine significantly enhances the nuclease resistance of the 3′ overhang in tissue culture medium.

The siRNAs of the invention can be targeted to any stretch of approximately about 18-30, preferably about 19-25 contiguous nucleotides in any of the target mRNA sequences (including the mRNA encoding a KZFP of the invention). Techniques for selecting target sequences for siRNA are well known in the art. Thus, the sense strand of the present siRNA comprises a nucleotide sequence identical to any contiguous stretch of about 18 to about 30 nucleotides in the target mRNA.

The siRNAs of the invention can be obtained using a number of techniques known to those of skill in the art. For example, the siRNAs can be chemically synthesized or recombinantly produced using methods known in the art. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), Qiagen (Hilden, Germany) and Cruachem (Glasgow, UK).

Alternatively, siRNA can also be expressed from recombinant circular or linear DNA plasmids using any suitable promoter. Suitable promoters for expressing siRNA of the invention from a plasmid include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the siRNA in a particular tissue or in a particular intracellular environment. The siRNA expressed from recombinant plasmids either can be isolated from cultured cell expression systems by standard techniques or can be expressed intracellularly.

The siRNAs of the invention can also be expressed from recombinant viral vectors intracellularly in cells. The recombinant viral vectors comprise sequences encoding the siRNAs of the invention and any suitable promoter for expressing the siRNA sequences.

Suitable promoters include, for example, the U6 or H1 RNA pol III promoter sequences and the cytomegalovirus promoter. Selection of other suitable promoters is within the skill in the art. The recombinant viral vectors of the invention can also comprise inducible or regulatable promoters for expression of the siRNA in a particular tissue.

Any RNA-targeting oligonucleotide can be considered as ASOs, although they may act through different mechanisms. Specific and strong recognition and binding to the mRNA target is accomplished through Watson-Crick base pairing, which may or may not be augmented by chemical modifications to the AON internucleotide phosphate linkages, backbone sugars, or nucleobases. The majority of ASOs can be divided into one of two groups: those that direct cleavage of the target mRNA, and those that alter mRNA translation without causing mRNA cleavage.

The present invention also contemplates a gene delivery vector, preferably in the form of a plasmid (circular or linear plasmid) or a vector, that comprises one or more nucleic acid(s) encoding an agent modulating the expression and/or activity of one or more KZFP of the invention.

Preferably, said one or more nucleic acid(s) encodes the siRNA, miRNA, piRNA, hnRNA, snRNA, sg RNA, CRISPR-based loss-of-function system, esiRNA, shRNA, and antisense oligonucleotide, or combination of one or more thereof, of the invention. Most preferably, the nucleic acid including an antisense oligonucleotide is selected from the group comprising an antisense oligonucleotide (ASO) and a modified ASO.

As used herein, a “vector” or a “gene delivery vector” is capable of transferring nucleic acid sequences to target cells (e.g., viral vectors, non-viral vectors, particulate carriers, and liposomes). Preferably, the nucleic acid is one or more nucleic acid(s) of the invention.

Suitable vectors include derivatives of SV40 and known bacterial plasmids, e.g., E. coli plasmids col E1, pCR1, pBR322, pMB9 and their derivatives, plasmids such as RP4; phage DNAs, e.g., the numerous derivatives of phage X, e.g., NM989, and other phage DNA, e.g., Ml 3 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like (for a review, Sung, Y., Kim, S. Recent advances in the development of gene delivery systems. Biomater Res 23, 8 (2019)).

Various viral vectors are used for delivering nucleic acids to cells in vitro or in vivo. Non-limiting examples are vectors based on Herpes Viruses, Pox-viruses, Adeno-associated virus, Lentivirus, and others. In principle, all of them are suited to deliver the expression cassette comprising an expressible nucleic acid molecule that codes for an agent modulating the expression and/or activity of one or more KZFP of the invention. In a preferred aspect, said viral vector is an adenoviral vector, preferably a replication competent adenovirus.

The present invention also contemplates a host cell comprising i) a plasmid or vector of the invention, or ii) one or more nucleic acid(s) encoding the siRNA, miRNA, piRNA, hnRNA, snRNA, sg RNA, CRISPR-based loss-of-function system, esiRNA, shRNA, and antisense oligonucleotide, or combination of one or more thereof, of the invention.

The gene delivery vector (e.g. plasmid or vector) comprising one or more nucleic acid(s) the siRNA, miRNA, piRNA, hnRNA, snRNA, sg RNA, CRISPR-based loss-of-function system, esiRNA, shRNA, and antisense oligonucleotide, or combination of one or more thereof, of the invention, or the one or more nucleic acid(s) of the invention, can be introduced to host cell via one or more methods known in the art. These one or more methods include, without limitation, microinjection, electroporation, calcium phosphate-mediated transfection, cationic transfection, liposome transfection, dendrimer transfection, heat shock transfection, nucleofection transfection, magnetofection, lipofection, optical transfection, proprietary agent-enhanced uptake of nucleic acids, and delivery via liposomes, immunoliposomes, virosomes, or artificial virions.

It will be appreciated that in the present method the modification following the introduction of the gene delivery vector (plasmid or vector), or the one or more nucleic acid(s) of the invention, to the host cell may occur ex vivo or in vitro, for instance in a cell culture and in some instances not in vivo. In other aspects, it may occur in vivo.

The present invention further contemplates one or more compositions. In one aspect, the composition comprises:
an agent modulating the expression and/or activity of one or more i) KZFP, ii) mRNA encoding a KZFP and/or iii) KZFP gene, of the invention, or
a plasmid or a vector of the invention, or
a host cell of the invention.

The present invention further contemplates one or more pharmaceutical compositions. In one aspect, the pharmaceutical composition comprises:
a therapeutically effective amount of an agent modulating the expression and/or activity of one or more i) KZFP, ii) mRNA encoding a KZFP and/or iii) KZFP gene, of the invention, or
a plasmid or a vector of the invention, or
a host cell of the invention,
   and a pharmaceutically acceptable carrier or diluent.

In one aspect, the pharmaceutical composition of the invention is for use in the treatment and/or prevention of cancer and/or cancer metastasis.

The term “therapeutically effective amount” as used herein means an amount of an agent of the invention high enough to significantly positively modify the symptoms and/or condition to be treated, but low enough to avoid serious side effects (at a reasonable risk/benefit ratio), within the scope of sound medical judgment. The therapeutically effective amount of the agent of the invention is selected in accordance with a variety of factors including type, species, age, weight, sex and medical condition of the patient; the severity of the condition to be treated; the route of administration; the renal and hepatic function of the patient. A physician of ordinary skill in the art can readily determine and prescribe the effective amount of the agent required to prevent, counter or arrest the progress of the cancer.

“Pharmaceutically acceptable carrier or diluent” means a carrier or diluent that is useful in preparing a pharmaceutical composition that is generally safe, non-toxic, and desirable, and includes carriers or diluents that are acceptable for human pharmaceutical use.

Such pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Pharmaceutically acceptable excipients include starch, glucose, lactose, sucrose, sodium stearate, glycerol monostearate, talc, sodium chloride, dried skim milk, glycerol, propylene glycol, water, ethanol and the like.

The pharmaceutical compositions may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include macrocrystalline cellulose, carboxymethyf cellulose sodium, polysorbate 80, phenyletbyl alcohol, chiorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

The pharmaceutical composition can also further comprise, or comprise the administration of, at least one additional anticancer agent or therapy selected from the group comprising radiotherapy, chemotherapy, immunotherapy, inhibitors of DNA methylation, targeted therapy and hormone therapy, or a combination of one of more thereof.

A chemotherapy of the present invention can concern as well agents that damage DNA and/or prevent cells from multiplying, such as genotoxins.

Genotoxins can be selected from the group comprising alkylating agents, antimetabolites, DNA cutters, DNA binders, topoisomerase poisons and spindle poisons. Examples of alkylating agents are lomustine, carmustine, streptozocin, mechlorethamine, melphalan, uracil nitrogen mustard, chlorambucil, cyclosphamide, iphosphamide, cisplatin, carboplatin, mitomycin, thiotepa, dacarbazin, procarbazine, hexamefhyl melamine, triethylene melamine, busulfan, pipobroman, mitotane and other platine derivatives or a combination of one of more thereof.

An example of DNA cutters is bleomycin.

Topoisomerases poisons can be selected from the group comprising topotecan, irinotecan, camptothecin sodium salt, daorubicin, doxorubicin, idarubicin, mitoxantrone teniposide, adriamycin and etoposide or a combination of one of more thereof.

Examples of DNA binders are dactinomycin and mithramycin whereas spindle poisons can be selected among the group comprising vinblastin, vincristin, navelbin, paclitaxel and docetaxel or a combination of one of more thereof.

A chemotherapy of the present invention can concern as well antimetabolites selected among the following non-limiting list of compounds: methotrexate, trimetrexate, pentostatin, cytarabin, ara-CMP, fludarabine phosphate, hydroxyurea, fluorouracyl, fioxuridine, chlorodeoxyadenosine, gemcitabine, thioguanine and 6-mercaptopurine or a combination of one of more thereof.

Radiotherapy refers to the use of high-energy radiation to shrink tumors and kill cancer cells. Examples of radiation therapy include, without limitation, external radiation therapy and internal radiation therapy (also called brachytherapy) or a combination of one of more thereof. External radiation therapy is most common and typically involves directing a beam of direct or indirect ionizing radiation to a tumor or cancer site. While the beams of radiation, the photons, the Cobalt or the particule therapy are focused to the tumor or cancer site, it is nearly impossible to avoid exposure of normal, healthy tissue. Energy source for external radiation therapy is selected from the group comprising direct or indirect ionizing radiation (for example: x-rays, gamma rays and particle beams or combination thereof).

Internal radiation therapy involves implanting a radiation-emitting source, such as beads, wires, pellets, capsules, etc., inside the body, at, or near to the tumor site. Energy source for internal radiation therapy is selected from the group of radioactive isotopes comprising: iodine (iodine125 or iodine131), strontium89, radioisotopes of phosphorous, palladium, cesium, indium, phosphate, or cobalt, and combination thereof. Such implants can be removed following treatment or left in the body inactive. Types of internal radiation therapy include, but are not limited to, interstitial, and intracavity brachytherapy (high dose rate, low dose rate, pulsed dose rate).

Another internal radiation therapy involves biological carriers of radioisotopes, such as with radio-immunotherapy wherein tumor-specific antibodies bound to radioactive material are administered to a patient. The antibodies bind tumor antigens, thereby effectively administering a dose of radiation to the relevant tissue.

Methods of administering radiation therapy are well known to those of skill in the art. Immunotherapy refers to the use of monoclonal antibodies, immune checkpoint inhibitors, cancer vaccines, cytokines, immunomodulators, Chimeric antigen receptor T (CAR-T) cells, T-cell receptor (TCR-T) cells or other adoptive cell therapies (such as e.g. Tumor Infiltrating Lymphocytes TILs). In a preferred aspect, the immunotherapy is selected from the group comprising monoclonal antibodies (e.g. targeting the protein CD20 such as Rituximab), immune checkpoint inhibitors or a combination of one or more thereof. Examples of immune checkpoint proteins targeted by inhibitors include PD-1/PD-L1, TIM-3, LAG-3 and CTLA-4/B7-1/B7-2.

Inhibitors of DNA methylation comprise DNA methyltransferase inhibitors (DNMTi). Non-limiting examples of DNMTi comprise nucleoside analogs such as, e.g. azacitide, decitabine, zebularine, guadecitabine, and 4′-thio-2′-deoxycytidine (TdCyd) as well as nonnucleoside analogs such as, e.g. procainamide, hydralazine, SGI-1027, MG98, and N-Phthaloyl-L-tryptophan (RG108). Up to now, the DNMT inhibitors are used to treat a variety of hematological tumors (for a review, see Hu, C., Liu, X., Zeng, Y. et al. DNA methyltransferase inhibitors combination therapy for the treatment of solid tumor: mechanism and clinical application. Clin Epigenet 13, 166 (2021)).

Targeted therapies are drugs that specifically target the changes inside cells that cause them to become cancer. Unlike chemotherapy drugs, which work by attacking rapidly growing cells in general (including cancer cells), these drugs target one or more specific proteins on or in cancer cells. Non-limiting examples of targeted therapies comprise kinase inhibitors, such as, e.g. Bruton's tyrosine kinase (BTK) inhibitors, Phosphoinositide 3-kinases (PI3K) inhibitors and SRC/ABL kinases.

Non-limiting examples of kinase inhibitors comprise drugs selected from ibrutinib, dasatinib and acalabrutinib.

The present invention further contemplates methods of treatment of cancer, and/or cancer metastasis.

In one aspect, the method of treatment of cancer, and/or cancer metastasis comprises:
(a) providing an agent of the invention and (b) administering said agent to a subject in need thereof.

Preferably, said agent is in the form of a pharmaceutical composition comprising a therapeutically effective amount of the agent as described herein.

In another aspect, the method comprises (a) modifying a host cell as described herein, and (b) reintroducing the host cell (e.g. single cell or population of cells), into the patient in need thereof, i.e. suffering from cancer and/or cancer metastasis. Preferably, said host cell is in the form of a pharmaceutical composition comprising a therapeutically effective amount of the host cell as described herein.

Administration of the agents and/or pharmaceutical compositions described herein may be accomplished by any acceptable method which allows the agents modulating the expression and/or activity of one or more i) KRAB-containing zinc finger protein (KZFP), ii) mRNA encoding a KZFP, and/or iii) KZFP gene to reach its target. The particular mode selected will depend of course, upon factors such as the particular formulation, the severity of the state of the subject being treated, and the dosage required for therapeutic efficacy. The actual effective amounts of agent (the “drug”) can vary according to the specific drug or combination thereof being utilized, the particular composition formulated, the mode of administration, and the age, weight, condition of the patient, and severity of the symptoms or condition being treated.

Any acceptable method known to one of ordinary skill in the art may be used to administer the agents and/or pharmaceutical compositions to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. The agents and/or pharmaceutical compositions can be injected intradermally for treatment cancer, for example.

The injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the agents and/or pharmaceutical compositions are encapsulated in liposomes.

Preferably, the agents and/or pharmaceutical compositions delivery systems are provided in a manner which enables tissue-specific uptake of the agents and/or pharmaceutical compositions delivery systems. Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

The agents and/or pharmaceutical compositions may be delivered using a bio-erodible implant by way of diffusion or by degradation of the polymeric matrix.

The administration of the agents and/or pharmaceutical compositions may be designed so as to result in sequential exposures to the agents and/or pharmaceutical compositions over a certain time period, for example, hours, days, weeks, months or years. This may be accomplished, for example, by repeated administrations of a formulation or by a sustained or controlled release delivery system in which the agents and/or pharmaceutical compositions is/are delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems suitable include, but are not limited to, time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include nonpolymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include, but are not limited to, erosional systems in which the agent and/or pharmaceutical composition contained in a formulation within a matrix (for example, as described in U.S. Pat. Nos. 4,452,775, 4,675,189, 5,736, 152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. The system may allow sustained or controlled release of the composition to occur, for example, through control of the diffusion or erosion/degradation rate of the formulation containing the agent and/or pharmaceutical composition. In addition, a pump-based hardware delivery system may be used for delivery.

Also envisioned is a tumor-specific delivery of an agent and/or pharmaceutical composition—coupled super-paramagnetic iron oxide nanoparticles as known in the art.

Further contemplated is a method of diagnosing cancer and/or cancer metastasis in a subject comprising:
(a) detecting, directly or indirectly, and measuring the level of transcription and/or expression and/or activity of one or more KZFP in a sample obtained from said subject;
(b) comparing the level of transcription and/or expression and/or activity of said one or more KZFP to a control biological sample for the same one or more KZFP;
wherein a differential transcription and/or expression and/or activity of one or more KZFP in said biological sample, relative to the level of corresponding said one or more KZFP in a control biological sample of a cancer-free subject, is indicative of the subject having cancer and/or cancer metastasis.

It is understood that the detection and measurement of the level of transcription and/or expression and/or activity of one or more KZFP in a sample obtained can be direct or indirect. For example, the abundance levels of mRNAs can be directly quantitated. Alternatively, the amount of one or more KZFP can be determined indirectly by measuring abundance levels of cDNAs, amplified RNAs or DNAs, or by measuring quantities or activities of RNAs, or other molecules that are indicative of the expression level of the one or more KZFP. Preferably, the detection and measurement of the level of transcription and/or expression and/or activity of one or more KZFP is determined indirectly by measuring abundance levels of cDNAs.

Transcripts (such as mRNAs), amplified RNAs or DNAs (such as cDNAs) can be detected and quantitated by a variety of methods including, but not limited to, microarray analysis, polymerase chain reaction (PCR), reverse transcriptase polymerase chain reaction (RT-PCR), Northern blot, serial analysis of gene expression (SAGE), immunoassay, and mass spectrometry, as well as any sequencing-based methods known in the art.

In one aspect, microarrays are used to measure the levels of one or more KZFP of the invention. An advantage of microarray analysis is that the expression of each of the one or more KZFP of the invention can be measured simultaneously, and microarrays can be specifically designed to provide a diagnostic expression profile for a particular disease or condition (e.g., cancer).

Microarrays are prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

Probes used in the methods of the invention are preferably immobilized to a solid support which may be either porous or non-porous. For example, the probes may be polynucleotide sequences which are attached to a nitrocellulose or nylon membrane or filter covalently at either the 3′ or the 5′ end of the polynucleotide. Such hybridization probes are well known in the art (see, e.g., Sambrook, et al., Molecular Cloning: A Laboratory Manual (3rd Edition, 2001). Alternatively, the solid support or surface may be a glass or plastic surface. In one embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel. In one embodiment, the microarray comprises a support or surface with an ordered array of binding (e.g., hybridization) sites or “probes” each representing one of the biomarkers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). Each probe is preferably covalently attached to the solid support at a single site. Microarrays can be made in a number of ways, of which several are described below. However they are produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. Microarrays are generally small, e.g., between 1 cm2 and 25 cm2; however, larger arrays may also be used, e.g., in screening arrays. Preferably, a given binding site or unique set of binding sites in the microarray will specifically bind (e.g., hybridize) to the product of a single gene in a cell (e.g., to a specific mRNA, or to a specific cDNA derived therefrom). However, in general, other related or similar sequences will cross hybridize to a given binding site.

As noted above, the “probe” to which a particular polynucleotide molecule specifically hybridizes contains a complementary polynucleotide sequence. The probes of the microarray typically consist of nucleotide sequences of no more than 1,000 nucleotides. In some embodiments, the probes of the array consist of nucleotide sequences of 10 to 1,000 nucleotides. In one aspect, the nucleotide sequences of the probes are in the range of 10-200 nucleotides in length and are genomic sequences of one species of organism, such that a plurality of different probes is present, with sequences complementary and thus capable of hybridizing to the genome of such a species of organism, sequentially tiled across all or a portion of the genome. In other aspects, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, or are 60 nucleotides in length. The probes may comprise DNA or DNA “mimics” (e.g., derivatives and analogues) corresponding to a portion of an organism's genome. In another aspect, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone (e.g., phosphorothioates).

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically, each probe on the microarray will be between 10 bases and 50,000 bases, usually between 300 bases and 1,000 bases in length. PCR methods are well known in the art, and are described, for example, in Innis et al., eds., PCR Protocols: A Guide To Methods And Applications, Academic Press Inc., San Diego, Calif. (1990); herein incorporated by reference in its entirety. It will be apparent to one skilled in the art that controlled robotic systems are useful for isolating and amplifying nucleic acids. An alternative, preferred means for generating polynucleotide probes is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., U.S. Pat. No. 5,539,083).

Probes are preferably selected using an algorithm that takes into account binding energies, base composition, sequence complexity, cross-hybridization binding energies, and secondary structure.

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

The probes are attached to a solid support or surface, which may be made, e.g., from glass, plastic (e.g., polypropylene, nylon), polyacrylamide, nitrocellulose, gel, or other porous or nonporous material. One method for attaching nucleic acids to a surface is by printing on glass plates, as known in the art. This method is especially useful for preparing microarrays of cDNA. A second method for making microarrays produces high-density oligonucleotide arrays. Techniques are known for producing arrays containing thousands of oligonucleotides complementary to defined sequences, at defined locations on a surface using photolithographic techniques for synthesis in situ (see, U.S. Pat. Nos. 5,578,832; 5,556,752; and 5,510,270; herein incorporated by reference in their entireties) or other methods for rapid synthesis and deposition of defined oligonucleotides. When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking, may also be used. In principle, any type of array known in the art, for example, dot blots on a nylon hybridization membrane could be used. However, as will be recognized by those skilled in the art, very small arrays will frequently be preferred because hybridization volumes will be smaller.

Microarrays can also be manufactured by means of an ink jet printing device for oligonucleotide synthesis, e.g., using the methods and systems described by Blanchard in U.S. Pat. No. 6,028,189. Specifically, the oligonucleotide probes in such microarrays are synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Microarrays manufactured by this ink jet method are typically of high density, preferably having a density of at least about 2,500 different probes per 1 cm2. The polynucleotide probes are attached to the support covalently at either the 3′ or the 5′ end of the polynucleotide.

KZFP polynucleotides which may be measured by microarray analysis can be expressed mRNAs or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one embodiment, the target polynucleotide molecules comprise RNA, including, but by no means limited to, total cellular RNA, poly(A)+ messenger RNA (mRNA) or a fraction thereof, cytoplasmic mRNA, or RNA transcribed from cDNA (i.e., cRNA; see, e.g., U.S. Pat. Nos. 5,545,522, 5,891,636, or 5,716,785). Methods for preparing total and poly(A)+ RNA are well known in the art, and are described generally, e.g., in Sambrook, et al., Molecular Cloning: A Laboratory Manual (3rd Edition, 2001). RNA can be extracted from a cell of interest using guanidinium thiocyanate lysis followed by CsCl centrifugation, a silica gel-based column (e.g., RNeasy (Qiagen, Valencia, Calif.) or StrataPrep (Stratagene, La Jolla, Calif.)), or using phenol and chloroform, as known in the art. Poly(A)+ RNA can be selected, e.g., by selection with oligo-dT cellulose or, alternatively, by oligo-dT primed reverse transcription of total cellular RNA. RNA can be fragmented by methods known in the art, e.g., by incubation with ZnC12, to generate fragments of RNA.

In one aspect, total RNA, mRNAs, or nucleic acids derived therefrom (such as cDNA), are isolated from a sample taken from a patient having cancer or a cancer tissue undergoing surgical and/or pharmacological therapies. KZFPs of the invention that are poorly expressed in particular cells may be enriched using normalization techniques known in the art. As described above, the KZFP polynucleotides can be detectably labeled at one or more nucleotides. Any method known in the art may be used to label the target polynucleotides. Preferably, this labeling incorporates the label uniformly along the length of the RNA, and more preferably, the labeling is carried out at a high degree of efficiency. For example, polynucleotides can be labeled by oligo-dT primed reverse transcription. Random primers (e.g., 9-mers) can be used in reverse transcription to uniformly incorporate labeled nucleotides over the full length of the polynucleotides. Alternatively, random primers may be used in conjunction with PCR methods or T7 promoter-based in vitro transcription methods in order to amplify polynucleotides.

The detectable label may be a luminescent label. For example, fluorescent labels, bioluminescent labels, chemiluminescent labels, and colorimetric labels may be used in the practice of the invention. Fluorescent labels that can be used include, but are not limited to, fluorescein, a phosphor, a rhodamine, or a polymethine dye derivative. Additionally, commercially available fluorescent labels including, but not limited to, fluorescent phosphoramidites such as FluorePrime (Amersham Pharmacia, Piscataway, N.J.), Fluoredite (Miilipore, Bedford, Mass.), FAM (ABI, Foster City, Calif.), and Cy3 or Cy5 (Amersham Pharmacia, Piscataway, N.J.) can be used. Alternatively, the detectable label can be a radiolabeled nucleotide.

In one aspect, KZFP polynucleotide molecules from a patient sample are labeled differentially from the corresponding polynucleotide molecules of a reference sample. The reference can comprise mRNAs from a normal biological sample (i.e., control sample, e.g., biopsy from a subject not having a cancer or a cancer tissue undergoing surgical and/or pharmacological therapies) or from a reference biological sample, (e.g., sample from a subject not having a cancer or a cancer tissue undergoing surgical and/or pharmacological therapies).

Nucleic acid hybridization and wash conditions are chosen so that the target polynucleotide molecules specifically bind or specifically hybridize to the complementary polynucleotide sequences of the array, preferably to a specific array site, wherein its complementary DNA is located. Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self-complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook, et al., Molecular Cloning: A Laboratory Manual (3rd Edition, 2001). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS). Particularly preferred hybridization conditions include hybridization at a temperature at or near the mean melting temperature of the probes (e.g., within 51º C., more preferably within 21° C.) in 1 M NaCl, 50 mM MES buffer (pH 6.5), 0.5% sodium sarcosine and 30% formamide.

When fluorescently labeled gene products are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously. Arrays can be scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are known in the art. Alternatively, a fiber-optic bundle, may be used to monitor RNA, mRNA, DNA or cDNA abundance levels at a large number of sites simultaneously.

As used herein, the wording “differential transcription and/or expression and/or activity of one or more KZFP” and their synonyms, which are used interchangeably, refer to one or more KZFP gene(s) or KZFP gene product(s) (e.g. transcripts or proteins) whose transcription and/or expression and/or activity is/are activated to a higher or lower level in a subject suffering from a disease, specifically cancer, relative to its/their transcription and/or expression and/or activity in a normal or control subject, or reference data. The terms also include KZFP genes or gene products whose transcription and/or expression and/or activity is altered to a higher or lower level at different stages of the same disease. It is also understood that a differentially transcripted and/or expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example.

Differential gene transcription and/or expression and/or activity may include a comparison of transcription and/or expression and/or activity between two or more KZFP genes or their gene products, or a comparison of the ratios of the transcription and/or expression and/or activity between two or more KZFP genes or their gene products, or even a comparison of two differently processed products of the same KZFP gene, which differ between normal subjects and subjects suffering from a disease, specifically cancer, or between various stages of the same disease. In particular, the reference (“control”) transcription and/or expression and/or activity level can be the transcription and/or expression and/or activity level of the KZFP gene product in a control sample. The control sample can be a normal sample, that is, a non-tumoural sample, preferably from the same tissue than the cancer sample, or a basal level of transcription and/or expression and/or activity.

The normal sample may be obtained from the subject affected with the cancer or from another subject, such as a normal or healthy subject, i.e. a subject who does not suffer from a cancer. Additionally, or alternatively, the control sample can be obtained from data repositories of cancer patient expression studies; accordingly, the control sample could be in this case a virtual sample obtained from such a data repository for a given cancer type.

Transcription and/or expression and/or activity levels obtained from cancer and normal samples may be normalized by using e.g. expression levels of proteins which are known to have stable expression such as RPLPO (acidic ribosomal phosphoprotein PO), TBP (TATA box binding protein), GAPDH (glyceraldehyde 3-phosphate dehydrogenase) or b-actin. Additionally, or alternatively, as it will be appreciated by a person skilled in the art, it is possible to use statistical methods based on whole-transcriptome expression distribution for normalization of cancer and control/normal samples.

Differential transcription and/or expression and/or activity includes both quantitative, as well as qualitative, differences in the temporal or cellular transcription and/or expression and/or activity pattern in a gene or its products among, for example, normal and diseased cells, or among cells which have undergone different disease events or disease stages. For the purpose of this description, “differential gene transcription and/or expression and/or activity” is considered to be present when there is at least a two-fold difference between the transcription and/or expression and/or activity of a given gene or gene product in normal and diseased subjects, or in various stages of disease development in a diseased subject.

In an aspect of the invention, the differential gene transcription and/or expression and/or activity of one or more KZFP corresponds to an upregulated expression, transcription and/or activity of said one or more KZFP.

Usually, the upregulated expression, transcription and/or activity of said one or more KZFP in a biological sample corresponds to an increase equal or superior to about 5%, preferably equal or superior to about 20%, more preferably equal or superior to about 40%, most preferably equal or superior to about 60%, more preferably equal or superior to about 500%, even more preferably equal or superior to about 1000%, in particular equal or superior to about 5000% when compared to the level of corresponding said one or more KZFP in a control biological sample of a cancer-free subject.

The biological sample is selected, in the context of the present application, from the group comprising whole blood, serum, plasma, semen, saliva, tears, urine, fecal material, sweat, buccal smears, skin, and cancer cells, or a combination of one or more of these biological samples.

The present invention also encompasses a method of stratifying a cancer in a subject comprising:
(a) detecting, directly or indirectly, and measuring the level of transcription and/or expression and/or activity of one or more KZFP in a sample obtained from said subject;
(b) comparing the level of transcription and/or expression and/or activity of said one or more KZFP to a control biological sample for the same one or more KZFP;
wherein an alteration in the transcription and/or expression and/or activity level of one or more one or more KZFP in said biological sample, relative to the level of corresponding said one or more KZFP in a control biological sample of a cancer-free subject, is indicative of the disease stage or grade.

In one aspect, the combination of one or more KZFP will comprise a combination of two or more, three or more, four or more, five or more, six or more, seven or more, ten or more, fifteen or more, twenty or more, twenty-five or more, thirty or more, forty or more, fifty or more, sixty or more, seventy or more, eighty or more, ninety or more, one hundred or more, or the complete set of the 110 KZFPs.

In one aspect, the KZFP will be selected from the group comprising ZNF200, ZNF671, ZNF586, ZNF343, ZNF473, ZNF614, ZNF7, ZNF547, ZNF773, ZNF776, ZKSCAN2, ZNF761, ZNF577, ZNF160, ZNF584, ZNF417, ZNF543, ZNF713, ZNF552, ZFP82, ZNF320, ZNF749, ZNF17, ZNF567, ZNF766, ZNF765, ZNF252P, ZNF420, ZNF615, ZNF121, ZNF544, ZNF583, ZNF587, ZNF814, ZNF805, ZNF134, ZNF850, and ZNF587B, or a combination of one or more thereof, e.g. two or more, three or more, four or more, five or more, six or more, seven or more, ten or more, fifteen or more, twenty or more, or the complete set of the 38 KZFPs.

In a preferred aspect, the KZFP will be selected from the group comprising ZNF107, ZNF121, ZNF200, ZNF239, ZNF473, ZNF547, ZNF558, ZNF587, ZNF695, ZNF7, ZNF700, ZNF749, ZNF766, ZNF783, ZNF789, ZNF814, ZNF841, ZNF8-ERVK3-1 and ZNF850, or a combination of one or more thereof, e.g. two or more, three or more, four or more, five or more, six or more, seven or more, ten or more, fifteen or more, or the complete set of the 19 KZFPs.

In a more preferred aspect, the KZFP will be selected from the group comprising ZNF121, ZNF200, ZNF473, ZNF547, ZNF587, ZNF7, ZNF749, ZNF766, ZNF814, and ZNF850, or a combination of one or more thereof, e.g. two or more, three or more, four or more, five or more, six or more, seven or more, nine or more, or the complete set of the 10 KZFPs.

Also encompassed is a method of determining the progression or regression, of a cancer in a subject suffering therefrom, said method comprising:
(a) detecting, directly or indirectly, and measuring the level of transcription and/or expression and/or activity of one or more KZFP in a sample obtained from said subject;
(b) periodically comparing the level of transcription and/or expression and/or activity of said one or more KZFP,
wherein an alteration in the level of transcription and/or expression and/or activity of one or more KZFP in a sample obtained from the same subject, relative to the level of transcription and/or expression and/or activity of one or more KZFP, determined previously, is indicative of the progression or regression of said cancer.

In an aspect of the invention, when the alteration in the transcription and/or expression and/or activity level(s) of one or more one or more KZFP corresponds to an enhanced transcription and/or expression and/or activity of said one or more KZFP, then this is indicative of the progression of said cancer.

In the same aspect of the invention, when the alteration in the transcription and/or expression and/or activity level(s) of one or more one or more KZFP corresponds to a decreased transcription and/or expression and/or activity of said one or more KZFP, then this is indicative of the regression of said cancer.

Alternatively, this method can also be used to determine the response to a cancer therapy. In this aspect of the invention, when the alteration in the transcription and/or expression and/or activity level of one or more one or more KZFP corresponds to an enhanced transcription and/or expression and/or activity of said one or more KZFP, then this is indicative that the cancer does not respond to said cancer therapy.

In case the subject suffering from cancer is determined to respond to the cancer therapy, the treatment is continued. Usually, the patient is predicted to respond when the level of corresponding transcription and/or expression and/or activity level of said one or more KZFP is downregulated.

In case the subject suffering from cancer is determined not to respond to the cancer therapy, the method further comprises a step of adapting the treatment. Usually, the patient is predicted not to respond when the level of corresponding transcription and/or expression and/or activity level of said one or more KZFP is upregulated or not significantly different to the level of the said one or more KZFP determined previously.

In one aspect, the step of adapting the treatment comprises not administering the envisioned treatment described herein, switching to another treatment, and/or adapting the dose of the treatment described herein.

These steps of continuing or adapting the cancer treatment are well within the skills and capacities of the medical doctors and medical staffs in charge of the follow-up of the treatment of the subject suffering from cancer.

In one aspect, the combination of one or more KZFP will comprise a combination of two or more, three or more, four or more, five or more, six or more, seven or more, ten or more, fifteen or more, twenty or more, twenty-five or more, thirty or more, forty or more, fifty or more, sixty or more, seventy or more, eighty or more, ninety or more, one hundred or more, or the complete set of the 110 KZFPs.

In one aspect, the KZFP will be selected from the group comprising ZNF200, ZNF671, ZNF586, ZNF343, ZNF473, ZNF614, ZNF7, ZNF547, ZNF773, ZNF776, ZKSCAN2, ZNF761, ZNF577, ZNF160, ZNF584, ZNF417, ZNF543, ZNF713, ZNF552, ZFP82, ZNF320, ZNF749, ZNF17, ZNF567, ZNF766, ZNF765, ZNF252P, ZNF420, ZNF615, ZNF121, ZNF544, ZNF583, ZNF587, ZNF814, ZNF805, ZNF134, ZNF850, and ZNF587B, or a combination of one or more thereof, e.g. two or more, three or more, four or more, five or more, six or more, seven or more, ten or more, fifteen or more, twenty or more, or the complete set of the 38 KZFPs.

In a preferred aspect, the KZFP will be selected from the group comprising ZNF107, ZNF121, ZNF200, ZNF239, ZNF473, ZNF547, ZNF558, ZNF587, ZNF695, ZNF7, ZNF700, ZNF749, ZNF766, ZNF783, ZNF789, ZNF814, ZNF841, ZNF8-ERVK3-1 and ZNF850, or a combination of one or more thereof, e.g. two or more, three or more, four or more, five or more, six or more, seven or more, ten or more, fifteen or more, or the complete set of the 19 KZFPs.

In a more preferred aspect, the KZFP will be selected from the group comprising ZNF121, ZNF200, ZNF473, ZNF547, ZNF587, ZNF7, ZNF749, ZNF766, ZNF814, and ZNF850, or a combination of one or more thereof, e.g. two or more, three or more, four or more, five or more, six or more, seven or more, nine or more, or the complete set of the 10 KZFPs.

Also encompassed is a method for classifying a cancer subject, the method comprising
(a) detecting, directly or indirectly, and measuring the level of transcription and/or expression and/or activity of one or more KZFP in a biological sample obtained from said subject, wherein said one or more KZFP is selected from the group listed in Table 1, or a variant or fragment thereof, and
(b) classifying the cancer subject as having a poor prognosis based on the presence and/or alteration of the transcription and/or expression and/or activity level(s) of the one or more KZFP in the biological sample.

The term “alteration” refers to a variation, either increase or decrease of said transcription and/or expression and/or activity level(s) when compared to a reference value (or reference range) or with the transcription and/or expression and/or activity level(s) determined previously, e.g. on the same cancer subject. Preferably, this alteration or variation is statistically significant.

As used herein, the term “poor prognosis” or “bad prognosis” refers to a patient or subject afflicted with cancer receiving or not a treatment that is likely to present metastasis, and/or that is likely to present cancer relapse, and/or that is likely to present short overall survival (OS), progression free survival (PFS) and/or metastasis.

In one aspect, the cancer subject is determined as having a poor prognosis when an alteration of the transcription and/or expression and/or activity level(s) of one or more KZFP, variant or fragment thereof, selected from the group comprising ZNF671, ZNF776, ZNF586, ZNF552, ZNF587B, ZNF417, ZNF107, ZNF121, ZNF200, ZNF239, ZNF473, ZNF547, ZNF558, ZNF587, ZNF695, ZNF7, ZNF700, ZNF749, ZNF766, ZNF783, ZNF789, ZNF814, ZNF841, and ZNF850, is detected and/or measured.

In a preferred aspect, the cancer subject is determined as having a poor prognosis when an alteration of the transcription and/or expression and/or activity level(s) of one or more KZFP, variant or fragment thereof, selected from the group comprising ZNF107, ZNF121, ZNF200, ZNF239, ZNF473, ZNF547, ZNF558, ZNF587, ZNF695, ZNF7, ZNF700, ZNF749, ZNF766, ZNF783, ZNF789, ZNF814, ZNF841, ZNF8-ERVK3-1 and ZNF850 is detected and/or measured.

In a more preferred aspect, the cancer subject is determined as having a poor prognosis when an alteration of the transcription and/or expression and/or activity level(s) of one or more KZFP, variant or fragment thereof, selected from the group comprising ZNF121, ZNF200, ZNF473, ZNF547, ZNF587, ZNF7, ZNF749, ZNF766, ZNF814, and ZNF850 is detected and/or measured.

Preferably, the alteration is an upregulated transcription, and/or upregulated expression and/or upregulated activity level(s) of one or more KZFP, variant or fragment thereof of Table 1.

In one aspect, the cancer subject is determined as having a poor prognosis when said one or more detected KZFP, variant or fragment thereof, is present in Table 1.

In one aspect, the cancer subject is determined as having a poor prognosis when the transcription and/or expression and/or activity level(s) of one or more KZFP, variant or fragment thereof of Table 1 is upregulated.

In one aspect, the cancer subject is suffering from lymphoma and is determined as having a poor prognosis when the transcription and/or expression and/or activity level(s) of one or more KZFP selected from the group comprising ZNF200, ZNF671, ZNF586, ZNF343, ZNF473, ZNF614, ZNF7, ZNF547, ZNF773, ZNF776, ZKSCAN2, ZNF761, ZNF577, ZNF160, ZNF584, ZNF417, ZNF543, ZNF713, ZNF552, ZFP82, ZNF320, ZNF749, ZNF17, ZNF567, ZNF766, ZNF765, ZNF252P, ZNF420, ZNF615, ZNF121, ZNF544, ZNF583, ZNF587, ZNF814, ZNF805, ZNF134, ZNF850, and ZNF587B, variant or fragment thereof, is upregulated.

In a preferred aspect, the cancer subject is suffering from lymphoma and is determined as having a poor prognosis when the transcription and/or expression and/or activity level(s) of one or more KZFP selected from the group comprising ZNF121, ZNF200, ZNF473, ZNF547, ZNF587, ZNF7, ZNF749, ZNF766, ZNF814, and ZNF850, variant or fragment thereof, is upregulated.

Also provided is a kit for performing a method according to the invention of for use in the treatment of cancer, said kit comprising
(a) one or more agents and/or pharmaceutical compositions, and
(b) instructions for use.

In some aspects, the kit comprises, alternatively or additionally, one or more oligonucleotide probes or primers specific to one or more target sequences within an mRNA encoding a KZFP, or a combination of KZFP, (i.e., oligonucleotide probes comprising a sequence complementary to a region of the KZFP of interest) selected from any of SEQ ID Nos of Table 1.

In some aspects, the kit comprises, alternatively or additionally, one or more oligonucleotide probes specific to one or more target sequences within an mRNA encoding a KZFP, or a combination of KZFP selected from the group comprising ZNF107, ZNF121, ZNF200, ZNF239, ZNF473, ZNF547, ZNF558, ZNF587, ZNF695, ZNF7, ZNF700, ZNF749, ZNF766, ZNF783, ZNF789, ZNF814, ZNF841, ZNF8-ERVK3-1 and ZNF850, a variant or a fragment thereof.

In some aspects, the kit comprises, alternatively or additionally, one or more oligonucleotide probes specific to one or more target sequences within an mRNA encoding a KZFP, or a combination of KZFP selected from the group comprising ZNF121, ZNF200, ZNF473, ZNF547, ZNF587, ZNF7, ZNF749, ZNF766, ZNF814, and ZNF850, a variant or a fragment thereof.

The disclosure is further illustrated by the following examples. The examples below are non-limiting and are merely representative of various aspects of the disclosure.

Also provided is one or more oligonucleotide probes or primers specific to one or more target sequences within an mRNA encoding a KZFP, or a combination of KZFP, (i.e., oligonucleotide probes comprising a sequence complementary to a region of the KZFP of interest) selected from any of SEQ ID Nos of Table 1. Preferably, the one or more oligonucleotide probes is/are specific to one or more target sequences within an mRNA encoding a KZFP, or a combination of KZFP selected from the group comprising ZNF107, ZNF121, ZNF200, ZNF239, ZNF473, ZNF547, ZNF558, ZNF587, ZNF695, ZNF7, ZNF700, ZNF749, ZNF766, ZNF783, ZNF789, ZNF814, ZNF841, ZNF8-ERVK3-1 and ZNF850, a variant or a fragment thereof. Most preferably, the one or more oligonucleotide probes is/are specific to one or more target sequences within an mRNA encoding a KZFP, or a combination of KZFP selected from the group comprising ZNF121, ZNF200, ZNF473, ZNF547, ZNF587, ZNF7, ZNF749, ZNF766, ZNF814, and ZNF850, a variant or a fragment thereof.

### EXAMPLES

### Example 1

### Primary Tumors Data

The RNA-seq dataset analyzed here has been granted access by the development assistance committee (DAC) of the University of Duke and accessed through The European Genome-phenome Archive (EGA) at the European Bioinformatics Institute. It includes RNA-seq data from 775 DLBCL samples sequenced on Illumina Hiseq 2500 platform generating 125 PE length reads with an average of 10 million reads per sample. The paired metadata used for survival analysis was extracted from the supplementary data of the study published by Reddy et al. Genetic and Functional Drivers of Diffuse Large B Cell Lymphoma. Reddy, Anupama et al. “Genetic and Functional Drivers of Diffuse Large B Cell Lymphoma.” Cell vol. 171, 2 (2017): 481-494.e15. doi:10.1016/j.cell.2017.09.027

### RNA-Seq Analysis

RNA-Seq bam files of the 775 DLBCL samples were downloaded from the EGA under the accession number EGAS00001002606. Read summarization on genes were generated using featureCounts with parameters -s 2 -t exon -g gene_id -Q 10. Sequencing depth normalization was performed using the TMM method as implemented in the voom function from the R package LIMMA from Bioconductor. One-hundred and forty-two samples with low coverage due to potential artefacts were omitted. The genes with an average expression<1 counts per million (CPM) across samples were filtered.

### RNA Extraction, Quantification, and Sequencing

Duplicates from two independent experiments were collected per sample. Total RNA was extracted with NucleoSpin RNA Plus (Macherey-Nagel) with an on-column deoxyribonuclease treatment. The sequencing libraries were generated using the TruSeq stranded mRNA sample preparation kit from Illumina. Libraries were then sequenced using the Illumina Hi-Seq technology with stranded 75-base single or paired-end reads. RNA-seq reads were mapped to the human genome (hg19) using HISAT2 (v2.1.0). Reads that were not uniquely mapped were discarded from the analysis using bamtools filter v2.4.1 with parameters -tag “NH: 1”.

### RNA-Seq Analysis

RNA-Seq bam files of the 775 DLBCL samples were downloaded from the EGA under the accession number EGAS00001002606. Read summarization on genes were generated using feature Counts with parameters -s 2 -t exon -g gene_id -Q 10. Sequencing depth normalization was performed using the TMM method as implemented in the voom function from the R package LIMMA from Bioconductor. One-hundred and forty-two samples with low coverage due to potential artefacts were omitted. The genes with an average expression<1 counts per million (CPM) across samples were filtered.

Gene-set enrichment analysis from differentially expressed genes in the KD experiments were performed using the Metascape webtool (www.metascape.org).

### Cell Culture

OCI-Ly7 and U2932 human lymphoma cell lines were obtained from DSMZ—German Collection of Microorganisms and Cell Cultures, Braunschweig, Germany (www.dsmz.de). The other cell lines used were obtained from ATCC collection, namely SU-DHL-4 (CRL-2957 ™) HL60 (CCL-240™), LoVo (CCL-229 ™), HCT116 (CCL-247 ™), HT-29 (HTB-38 ™)293T (CRL-11268 ™), K562 (CCL-243 ™). HBL-1 were obtained from ABM (CAT No T8204). All lymphoma cell lines were grown in RPMI 1640 (Gibco®) containing 10-20% fetal calf serum (FCS) and 1% penicillin, with the exception of OCI-Ly7 which were grown in IMDM containing 20% FCS and 1% penicillin. All cell lines were cultured at 37° C. and 5% CO₂. Cells were transduced, selected with 2-3 ug/mL puromycin and collected after 6 days for RNA-sequencing.

### Plasmids and Lentiviral Vectors

pLKO.puro shRNA vectors were used for ZNF586, ZNF587 and ZNF417 knock downs. The hairpin sequences against ZNF417/587 (GCAGCATATTGGAGAGAAATT—SEQ ID No 243; AGTCGAAAGAGCAGCCTTATT—SEQ ID No. 244) were designed using the Genetic Perturbation Portal of the Broad Institute and inserted with Age I/Eco RI into pLKO.1.puro LV (TRCN0000018002; Sigma-Aldrich). The short hairpin RNAs (shRNAs) targeting the sequence ZNF586 (TRCN0000015373 and TRCN0000015376) were obtained from Sigma-Aldrich. Lentiviral vectors production protocols are detailed at http://tronolab.epfl.ch. The cell proliferation after LV transduction was determined using the 3-[4,5-dimethylthiazol-2-yl]-2,5-diphenyltetrazolium bromide (MTT) assay. The cells were transduced at a multiplicity of infection (MOI) of 20 in every experiment.

### Results

Diffuse large B cell lymphoma (DLBCL) is the most frequently diagnosed aggressive non-Hodgkin lymphoma (NHL). It comprises several histological variants, which can be grouped into three main molecular subtypes based on gene expression signatures: germinal center (GCB), activated B-cell (ABC) and unclassified (UNCL) DLBCL. The distinction between these three groups holds prognostic and therapeutic value, ABC DLBCL has a worse prognosis than its GCB or UNCL counterparts as it is more frequently refractory to first line standard treatments and prone to relapse. The standard of care for first-line therapy in DLBCL is the R-CHOP (rituximab, cyclophosphamide, doxorubicin, vincristine and prednisone) regimen. Research on drug resistance has so far mainly focused on genetic alterations, but recent data points to the role of epigenetic changes. For instance, anthracycline-resistance has been linked to silencing of the transcription factor SMAD1 by DNA methylation of its promoter, resulting in impaired activation of DNA damage response genes. Accordingly, inhibitors of DNA methylation, such as 5-azacytidine, are currently coupled with R-CHOP in some clinical trials. Although advances in therapy over the last 20 years have led to improvements in cancer survival, additional strategies are still urgently needed.

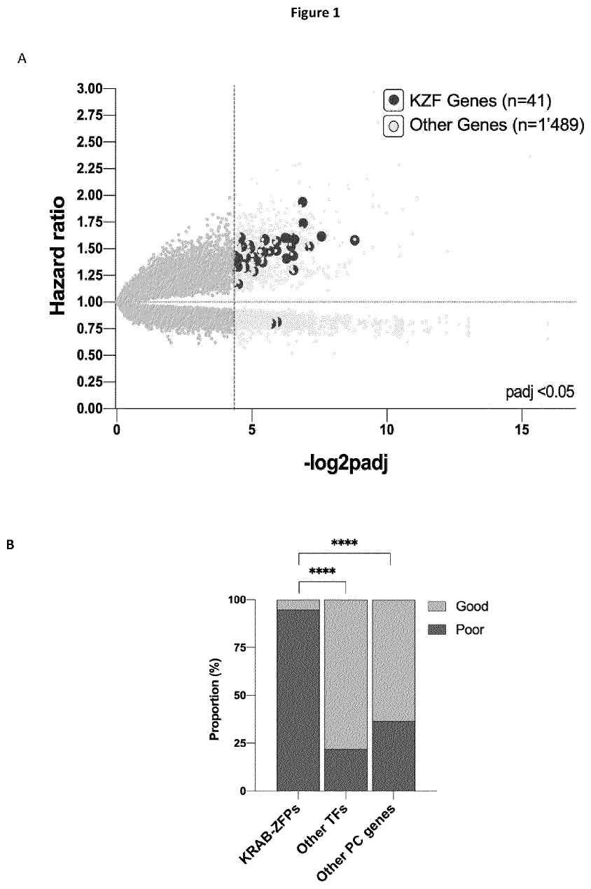

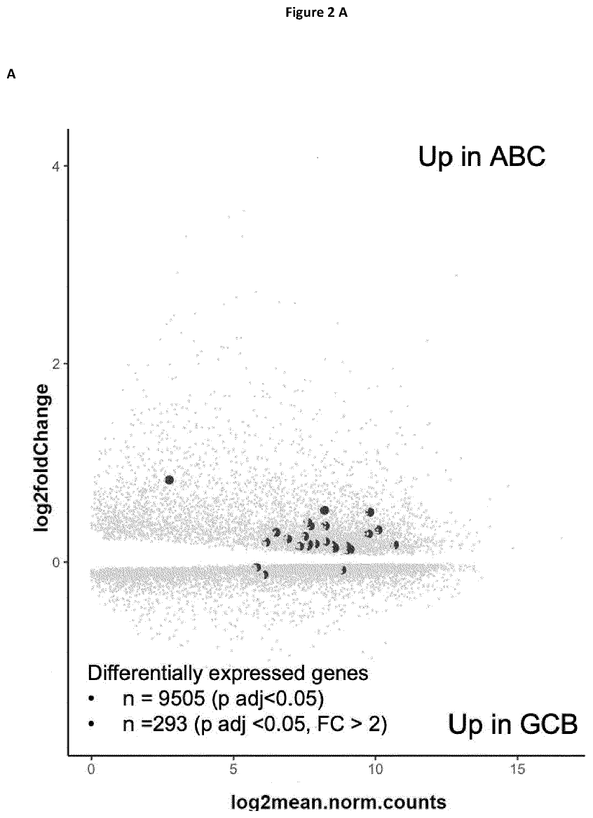

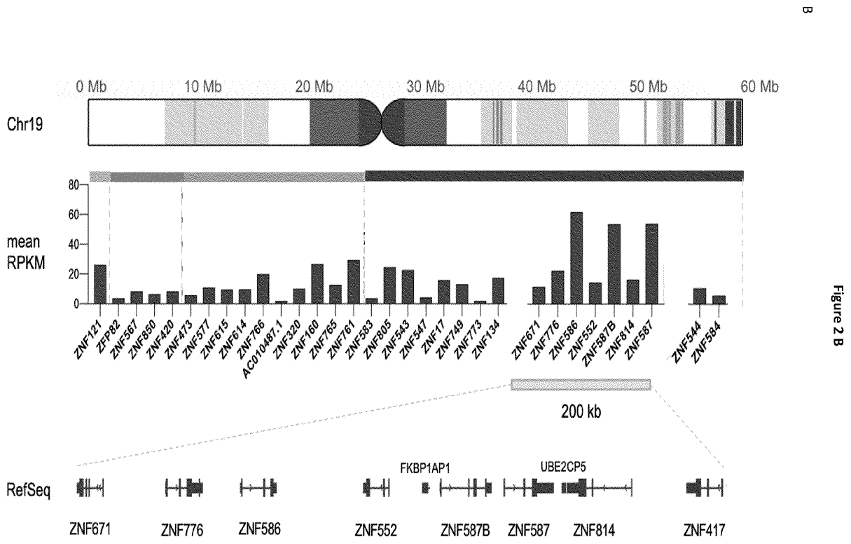

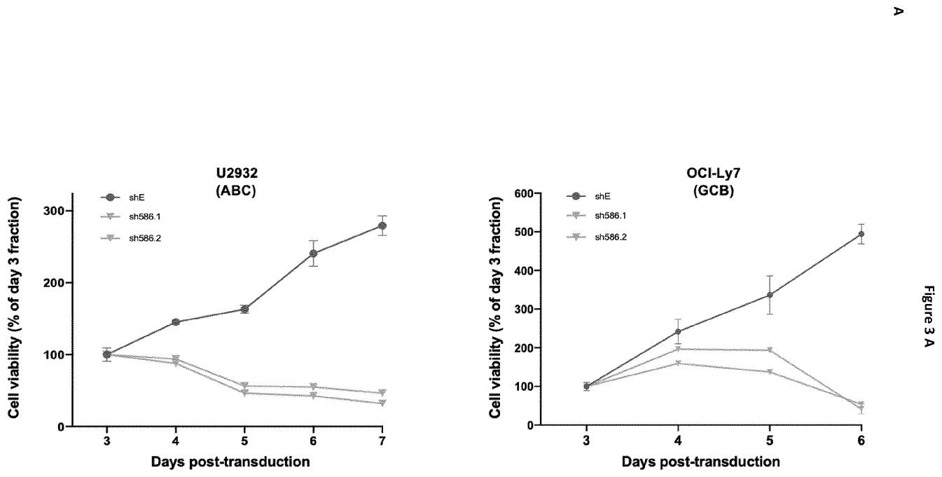

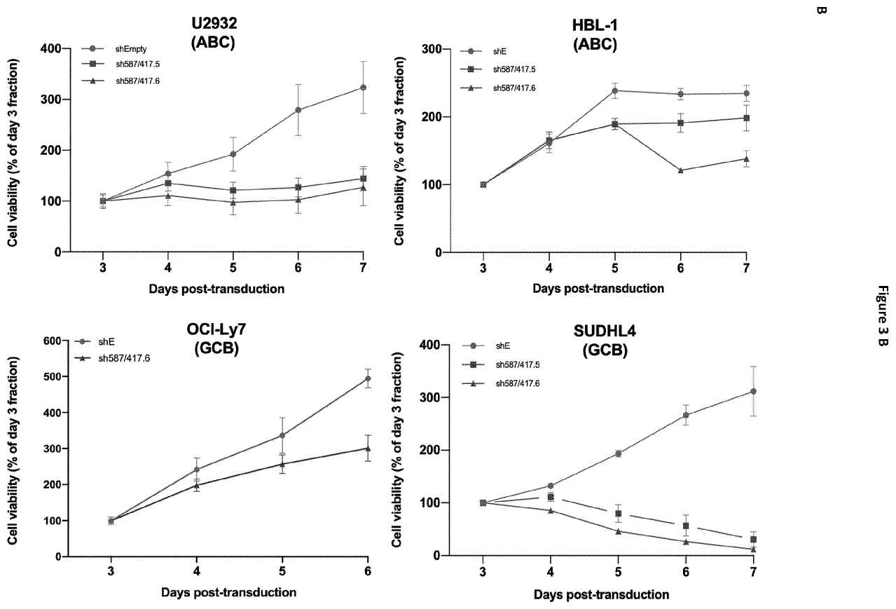

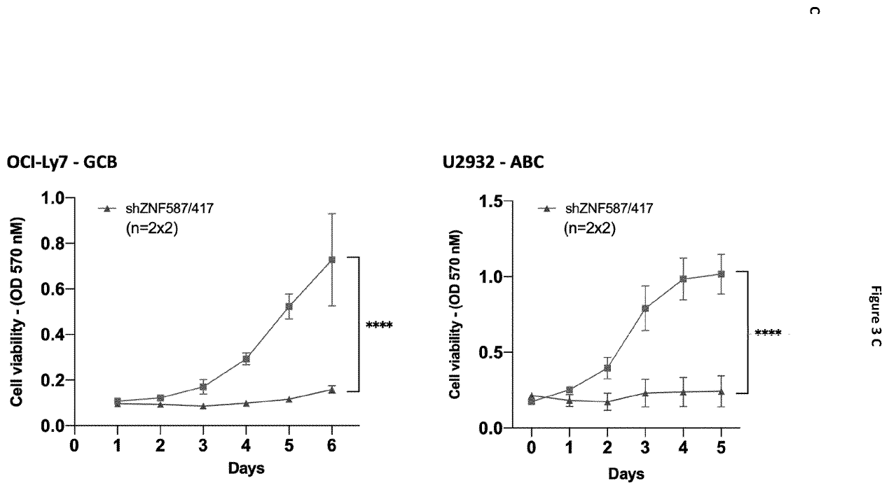

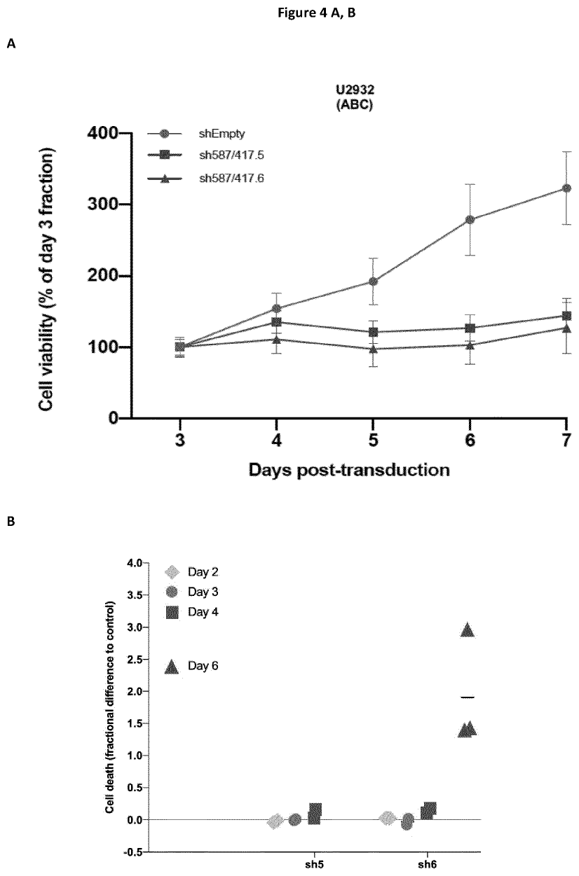

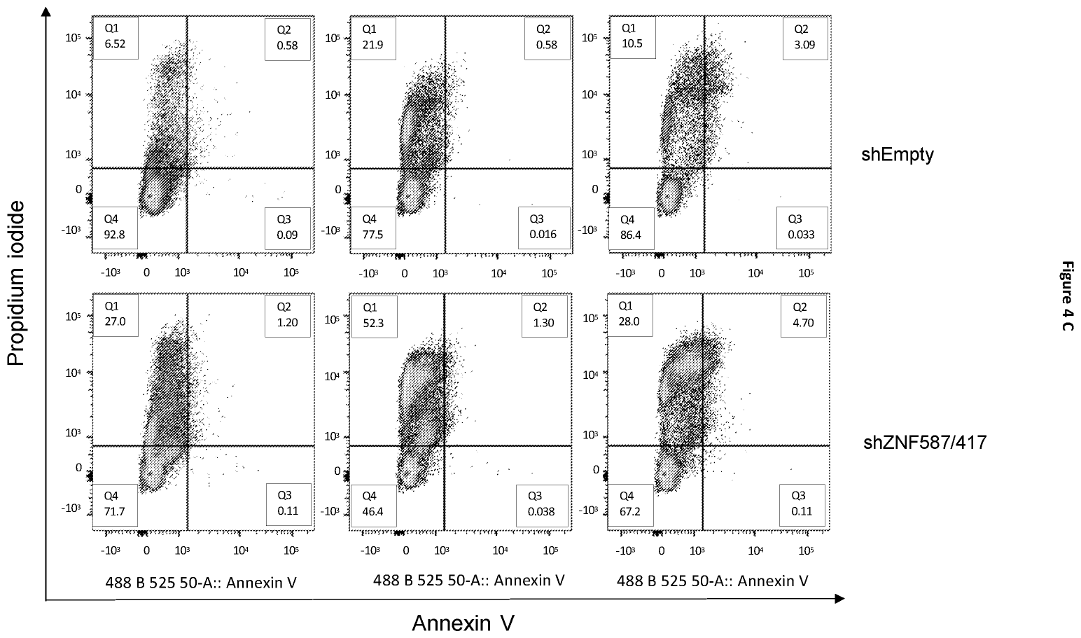

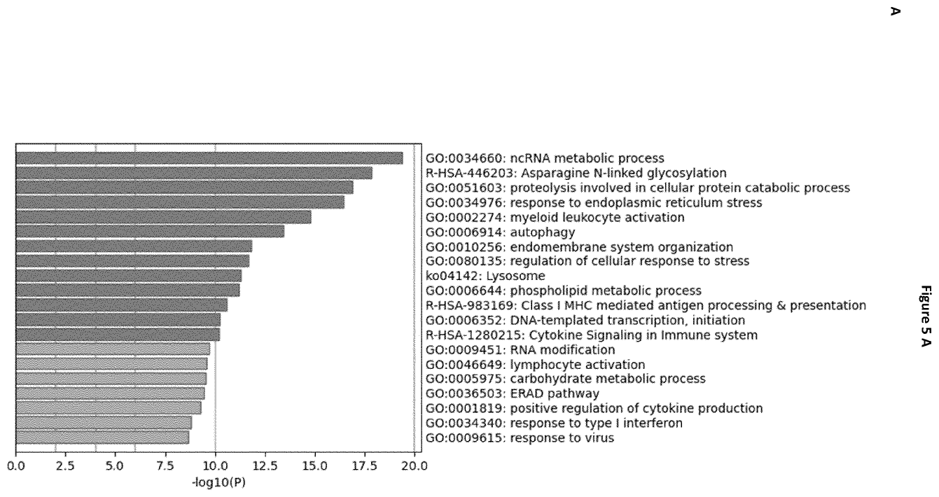

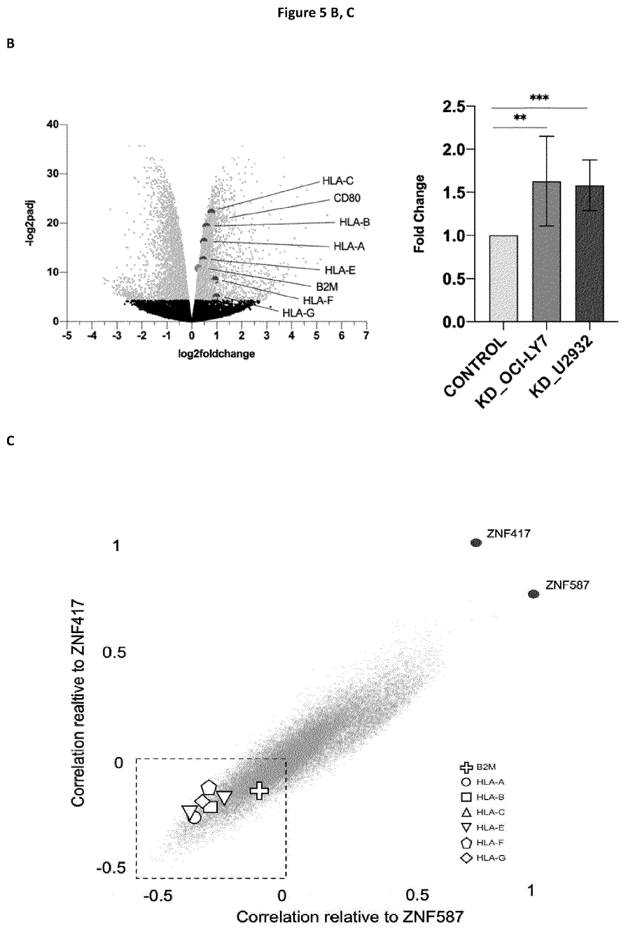

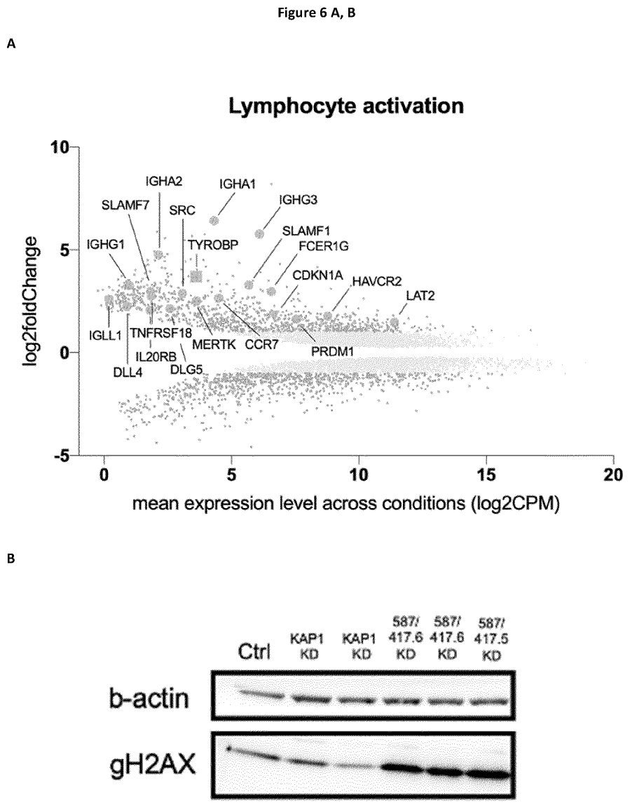

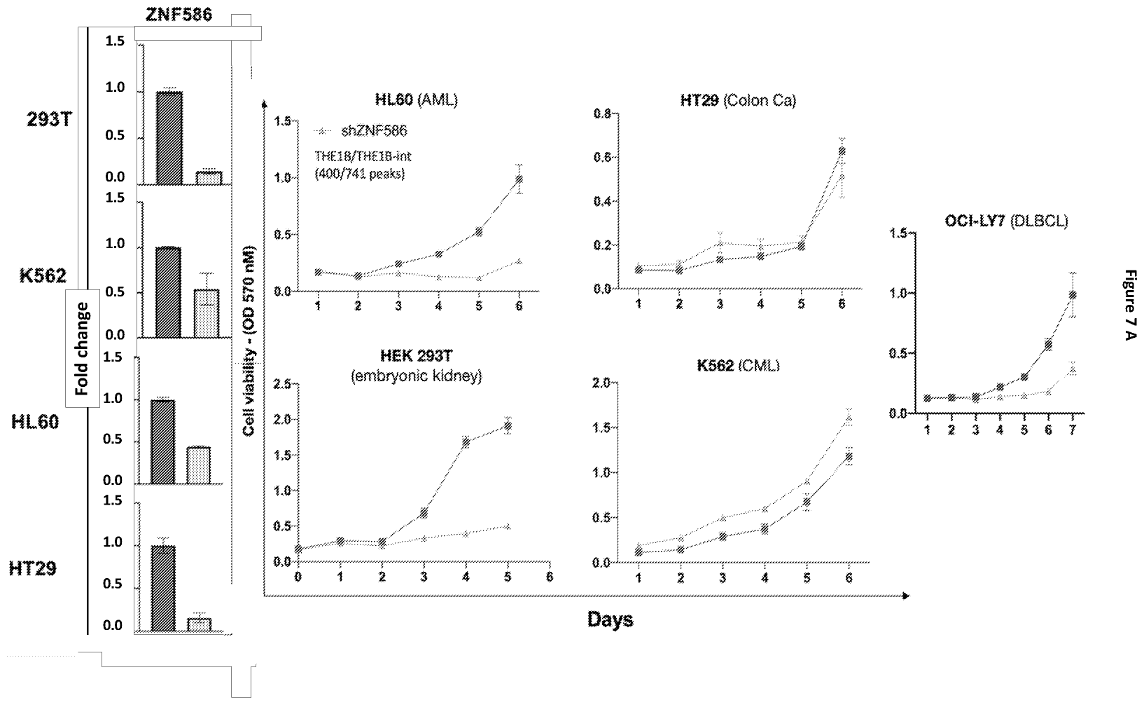

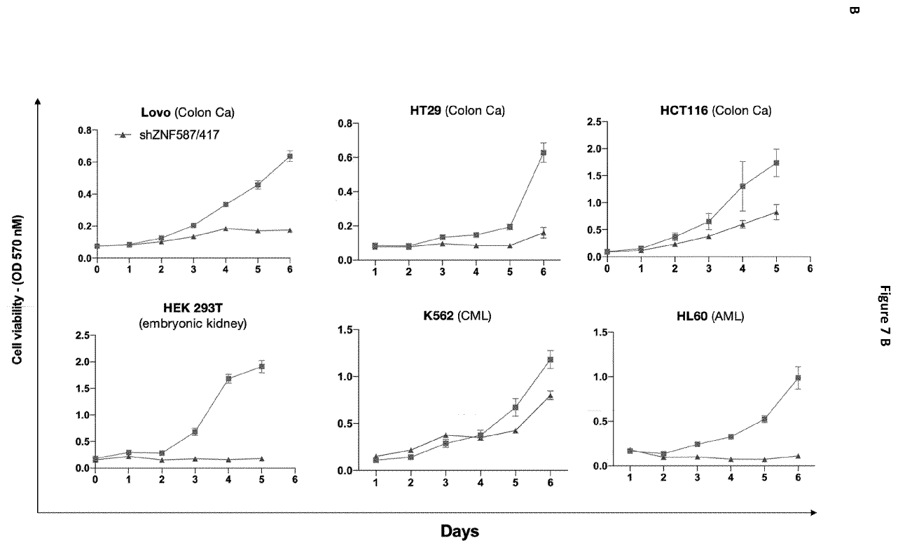

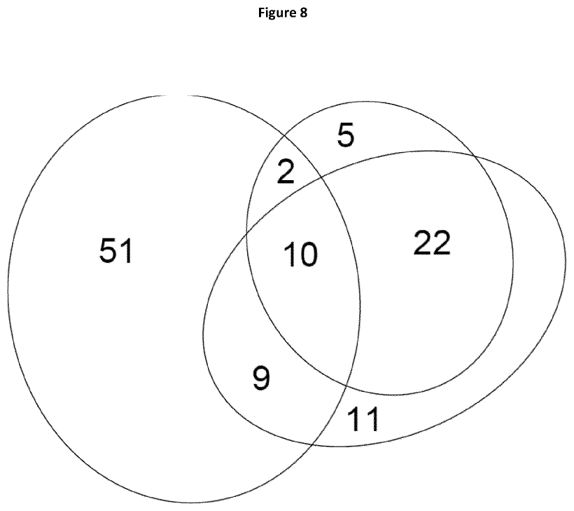

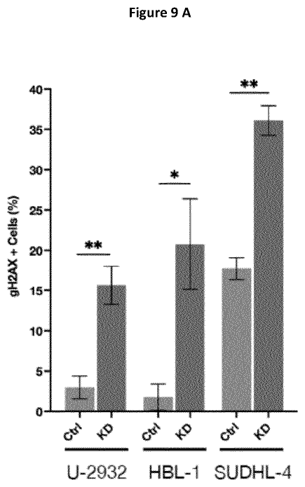

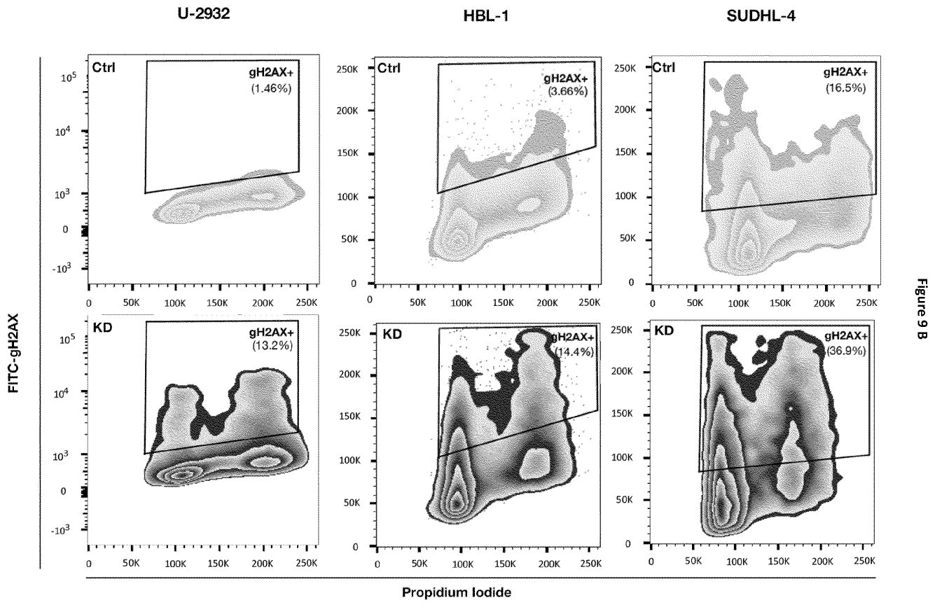

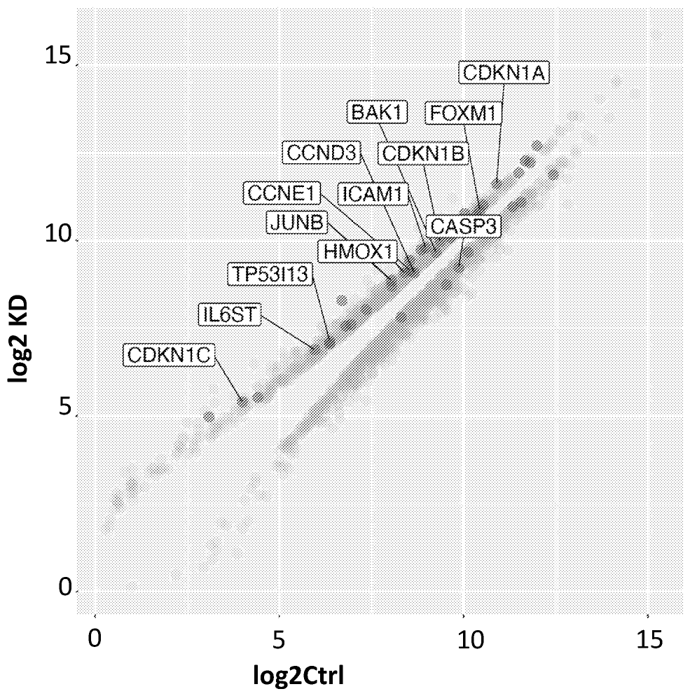

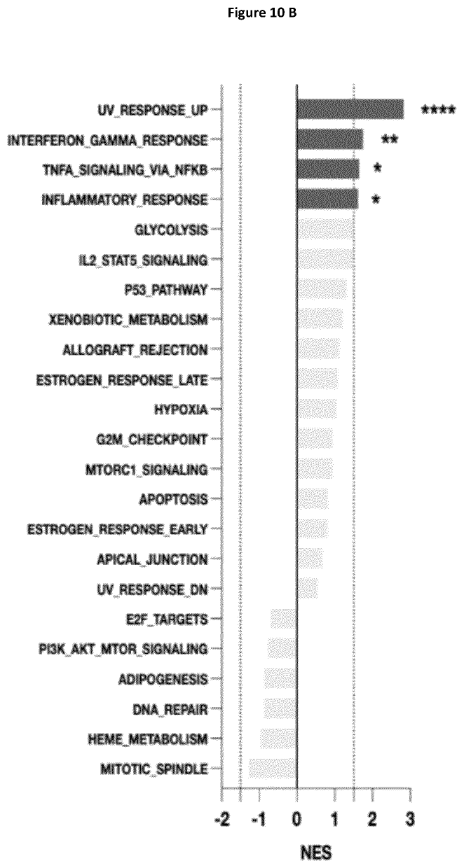

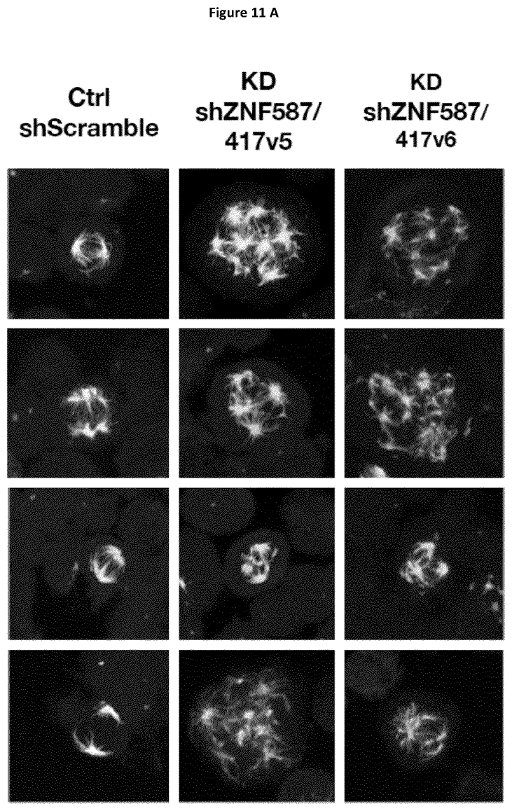

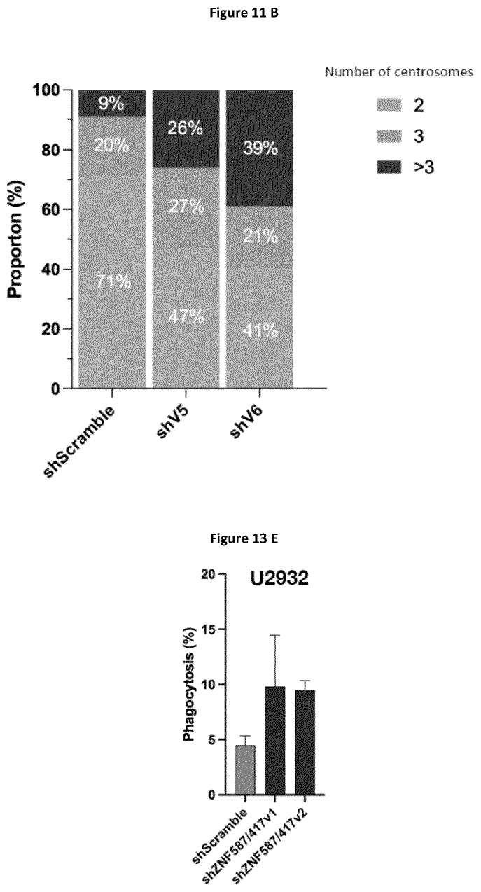

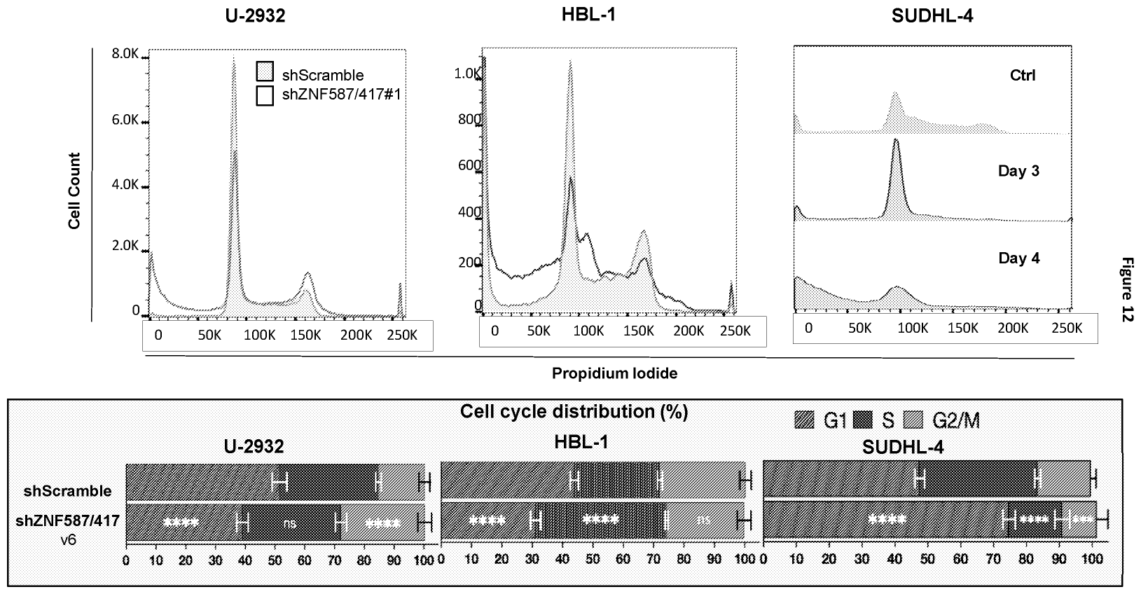

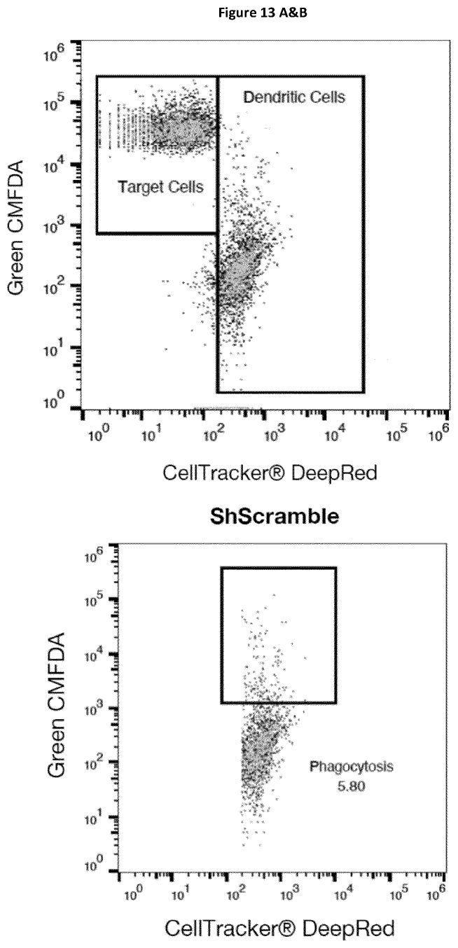

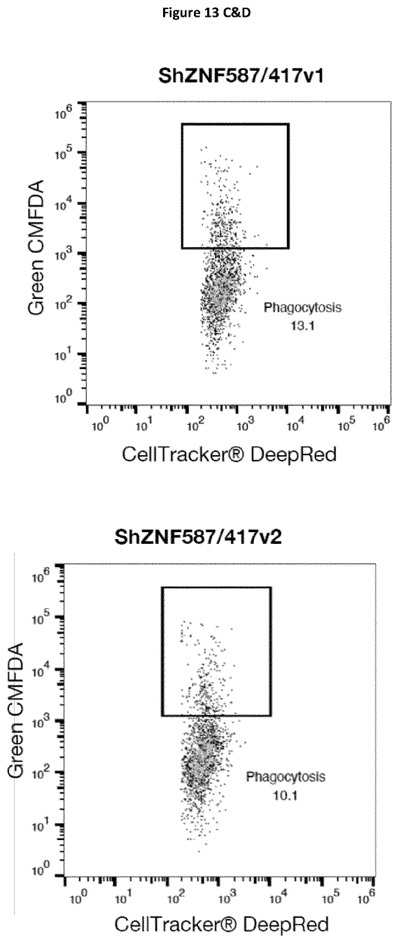

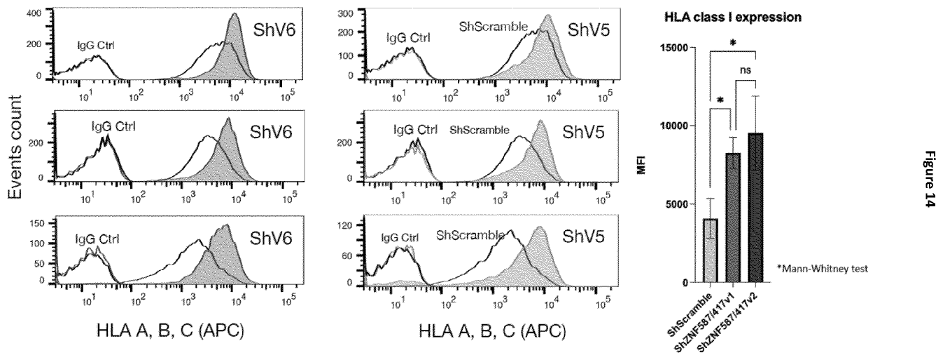

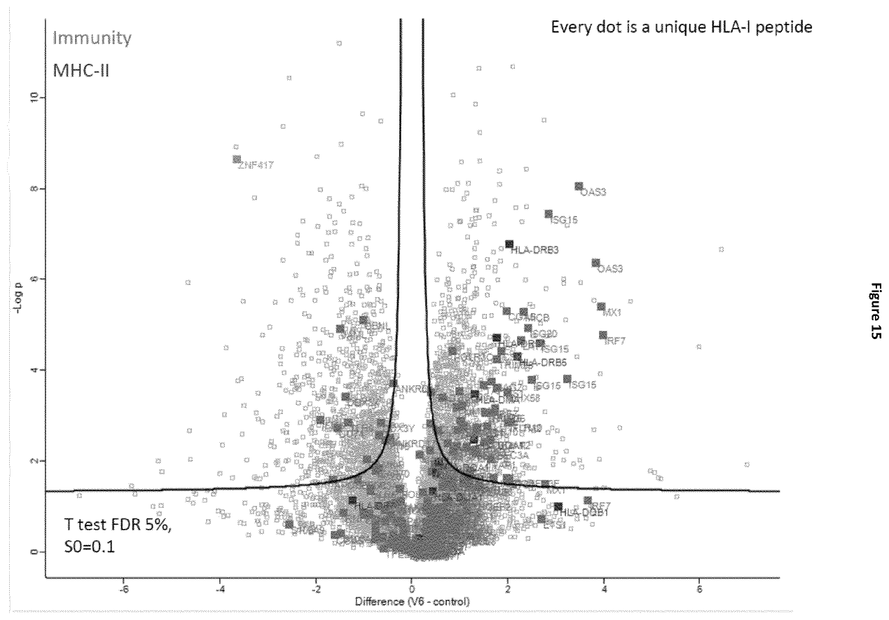

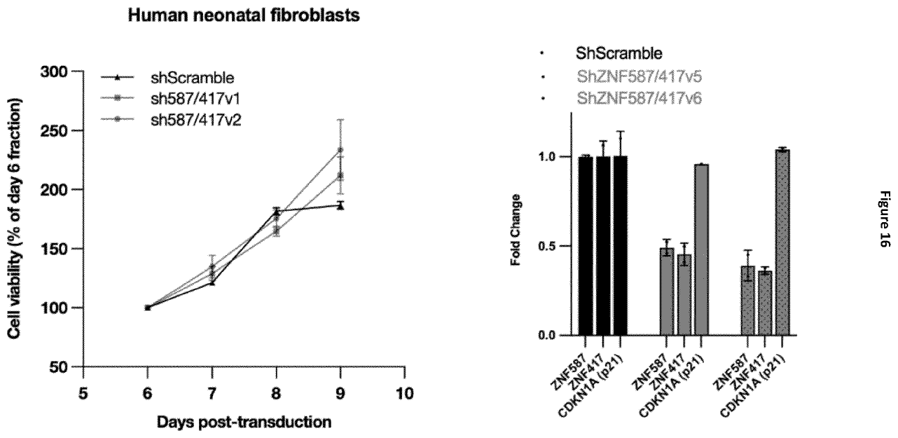

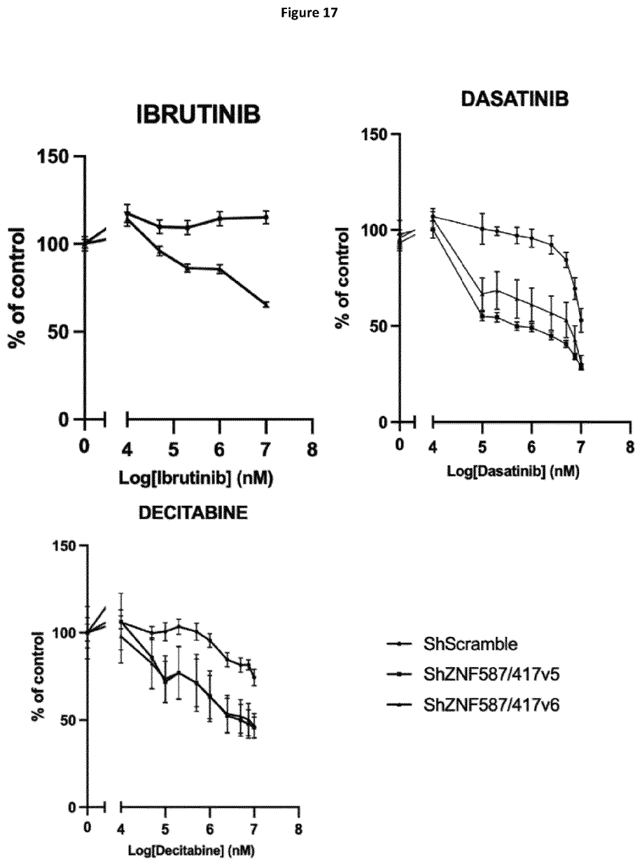

We used our transcriptome analytical pipeline on a cohort of 630 DCBL patients (FIG. 1). Out of 23,560 genes, 1,530 were significantly associated with survival. Amongst these, 41 encoded for KZFPs (dark dots), which were associated with an increased risk of death compared with other TFs and protein coding genes.

Of 39 KZFP genes associated with poor prognosis, 38 were upregulated in the ABC DCBL subtype, 82% of which located on the long arm of chromosome 19 (Chr19q), a region frequently amplified in forms of DLBCL associated with disease relapse.

ZNF586, ZNF587 and its paralog ZNF417 were amongst the most highly expressed genes of this region, along with 5 others KZFP genes (namely ZNF587B, ZNF814, ZNF552, ZNF776 and ZNF671) also associated to poor prognosis clustered over a 200 kb segment.

Gene-silencing experiments of ZNF586, ZNF587 and ZNF417, across a panel of DLBCL cell lines, demonstrated a stringent proliferation arrest phenotype, which was also noticed in leukemia and colon cancer cell lines.

These experiments also revealed that ZNF586, ZNF587 and ZNF417 depletion was able to trigger a necrotic form of cell death, the mechanisms of which is currently being investigated. Transcriptomic analysis of arrested cells revealed enrichments in pathways implicated in proteolysis, endoplasmic reticulum-associated protein degradation (ERAD), antigen processing, major histocompatibility complex (MCH) class I presentation and cytokine production, suggesting increased sensitivity to immune recognition and killing. Additionally, proteome analysis of cell supernatant detected the presence of CXCL10, a chemokine implicated in T-cell, NK cell and macrophage migration, also suggesting that maneuvers aimed at blocking the effect of the KZFPs might enhance anti-tumor immune responses.

Based on these results, ZNF586, ZNF587 and ZNF417, along with other members of their 200 kb cluster, are new potential targets for the treatment of aggressive non-Hodgkin lymphoma and other tumors.

### Example 2

A transcriptome-based univariate Cox proportional regression analysis was conducted to identify genes whose expression levels were associated with poor survival across 31 TCGA tumor types. This table contains the KZFP gene hits that were associated with a decrease in survival probability as defined by a positive score (hazard ratio coefficients) and a false-discovery rate (FDR)<0.05 for each tumor type.

| Name | cancer | n | HR | Pvalue | FDR | cancer | n | HR | Pvalue | FDR |
|---|---|---|---|---|---|---|---|---|---|---|
| ZNF695 | KIRP | 287 | 1.4 | 1.00E−04 | 0.004938 | KIRC | 529 | 1.2 | 7.00E−04 | 0.0020634 |
| ZNF558 | LGG | 510 | 1.6 | 0 | 0 | KICH | 64 | 5.4 | 0 | 0 |
| ZNF239 | LIHC | 370 | 1.4 | 0 | 0 | KIRP | 287 | 1.5 | 5.00E−04 | 0.0131667 |
| ZNF700 | LGG | 510 | 1.5 | 0 | 0 | KIRC | 529 | 1.4 | 0 | 0 |
| ZNF587 | LGG | 510 | 1.3 | 0.0061 | 0.01746 | KIRC | 529 | 1.3 | 0.001 | 0.0027431 |
| ZNF7 | KIRP | 287 | 1.5 | 0.0035 | 0.044597 | KIRC | 529 | 1.3 | 4.00E−04 | 0.0012742 |
| ZNF783 | KIRC | 529 | 1.4 | 0 | 0 | ACC | 79 | 1.8 | 0.0017 | 0.0373056 |
| ZNF107 | LGG | 510 | 1.2 | 0.0016 | 0.006257 | KIRC | 529 | 1.3 | 4.00E−04 | 0.0012742 |
| ZNF547 | LGG | 510 | 1.3 | 0.003 | 0.009958 | KIRC | 529 | 1.2 | 0.0253 | 0.0456324 |
| ZNF789 | LGG | 510 | 1.3 | 0.0021 | 0.007825 | KIRC | 529 | 1.3 | 4.00E−04 | 0.0012742 |
| ZNF841 | LGG | 510 | 1.3 | 0.0029 | 0.009958 | KIRC | 529 | 1.3 | 1.00E−04 | 0.0003873 |
| ZNF473 | LGG | 510 | 1.5 | 0 | 0 | MESO | 85 | 1.5 | 6.00E−04 | 0.0131667 |
| ZNF749 | LGG | 510 | 1.4 | 1.00E−04 | 0.000556 | MESO | 85 | 1.6 | 0.0019 | 0.0326304 |
| RBAK | LGG | 510 | 1.5 | 0 | 0 | NA | NA | NA | NA | NA |
| ZFP30 | LGG | 510 | 1.3 | 0.0066 | 0.018104 | NA | NA | NA | NA | NA |
| ZFP69B | LIHC | 370 | 1.5 | 0 | 0 | NA | NA | NA | NA | NA |
| ZNF121 | PRAD | 496 | 2.1 | 4.00E−04 | 0.0316 | NA | NA | NA | NA | NA |
| ZNF200 | LGG | 510 | 1.4 | 0 | 0 | NA | NA | NA | NA | NA |
| ZNF251 | KIRC | 529 | 1.4 | 0 | 0 | NA | NA | NA | NA | NA |
| ZNF282 | ACC | 79 | 2 | 2.00E−04 | 0.011286 | NA | NA | NA | NA | NA |
| ZNF283 | LGG | 510 | 1.4 | 4.00E−04 | 0.001881 | NA | NA | NA | NA | NA |
| ZNF337 | KIRC | 529 | 1.3 | 2.00E−04 | 0.000725 | NA | NA | NA | NA | NA |
| ZNF354A | KIRC | 529 | 1.2 | 0.0259 | 0.046502 | NA | NA | NA | NA | NA |
| ZNF419 | LGG | 510 | 1.2 | 0.013 | 0.033783 | NA | NA | NA | NA | NA |
| ZNF432 | LGG | 510 | 1.5 | 0 | 0 | NA | NA | NA | NA | NA |
| ZNF485 | LIHC | 370 | 1.4 | 3.00E−04 | 0.0395 | NA | NA | NA | NA | NA |
| ZNF496 | ACC | 79 | 2 | 0 | 0 | NA | NA | NA | NA | NA |
| ZNF514 | KIRC | 529 | 1.3 | 0.0031 | 0.007332 | NA | NA | NA | NA | NA |
| ZNP517 | KIRC | 529 | 1.3 | 7.00E−04 | 0.002063 | NA | NA | NA | NA | NA |
| ZNF525 | LGG | 510 | 1.5 | 0 | 0 | NA | NA | NA | NA | NA |
| ZNF530 | LGG | 510 | 1.6 | 0 | 0 | NA | NA | NA | NA | NA |
| ZNP550 | LGG | 510 | 1.3 | 0.003 | 0.009958 | NA | NA | NA | NA | NA |
| ZNF707 | KIRC | 529 | 1.3 | 2.00E−04 | 0.000725 | NA | NA | NA | NA | NA |
| ZNF746 | KIRC | 529 | 1.3 | 0 | 0 | NA | NA | NA | NA | NA |
| ZNF766 | LGG | 510 | 1.3 | 0.0031 | 0.010037 | NA | NA | NA | NA | NA |
| ZNF80 | KIRC | 529 | 1.2 | 0.0028 | 0.006785 | NA | NA | NA | NA | NA |
| ZNF814 | KIRC | 529 | 1.3 | 2.00E−04 | 0.000725 | NA | NA | NA | NA | NA |
| ZNF850 | LGG | 510 | 1.5 | 0 | 0 | NA | NA | NA | NA | NA |
| ZSCAN22 | LGG | 510 | 1.5 | 0 | 0 | NA | NA | NA | NA | NA |
| ZNF93 | KIRP | 287 | 1.4 | 0.0033 | 0.044597 | SKCM | 103 | 2.1 | 1.00E−04 | 0.0395 |
| ZNF18 | LGG | 510 | 1.3 | 0.0049 | 0.014553 | KIRC | 529 | 1.4 | 0 | 0 |
| ZNF202 | KIRC | 529 | 1.3 | 2.00E−04 | 0.000725 | ACC | 79 | 2 | 0 | 0 |
| ZNF117 | KICH | 64 | 3.2 | 0 | 0 | KIRC | 529 | 1.4 | 0 | 0 |
| ZNF600 | LGG | 510 | 1.7 | 0 | 0 | KIRC | 529 | 1.3 | 3.00E−04 | 0.0010042 |
| ZNF83 | LGG | 510 | 1.3 | 0.0012 | 0.004837 | KIRC | 529 | 1.4 | 0 | 0 |
| ZNF71 | LGG | 510 | 1.4 | 0 | 0 | MESO | 85 | 1.9 | 0 | 0 |
| ZNF101 | LGG | 510 | 1.5 | 0 | 0 | UVM | 80 | 1.7 | 0.0099 | 0.0420484 |
| ZNF267 | LGG | 510 | 1.4 | 2.00E−04 | 0.001039 | UVM | 80 | 1.7 | 0.0096 | 0.0416703 |
| ZNF480 | LGG | 510 | 1.6 | 0 | 0 | UVM | 80 | 1.9 | 0.0022 | 0.0125942 |
| HKR1 | LGG | 510 | 1.3 | 0.0039 | 0.011942 | NA | NA | NA | NA | NA |
| ZFP28 | LGG | 510 | 1.3 | 0.0022 | 0.008046 | NA | NA | NA | NA | NA |
| ZFP69 | KIRC | 529 | 1.2 | 0.0095 | 0.01975 | NA | NA | NA | NA | NA |
| ZIK1 | LGG | 510 | 1.4 | 1.00E−04 | 0.000556 | NA | NA | NA | NA | NA |
| ZKSCAN5 | KICH | 64 | 2.8 | 5.00E−04 | 0.0395 | NA | NA | NA | NA | NA |
| ZNF112 | LGG | 510 | 1.3 | 0.0085 | 0.02284 | NA | NA | NA | NA | NA |
| ZNF12 | LGG | 510 | 1.4 | 2.00E−04 | 0.001039 | NA | NA | NA | NA | NA |
| ZNF132 | LGG | 510 | 1.3 | 0.0029 | 0.009958 | NA | NA | NA | NA | NA |
| ZNF133 | KIRC | 529 | 1.3 | 3.00E−04 | 0.001004 | NA | NA | NA | NA | NA |
| ZNF134 | LGG | 510 | 1.4 | 0 | 0 | NA | NA | NA | NA | NA |
| ZNF136 | LGG | 510 | 1.3 | 0.0036 | 0.011376 | NA | NA | NA | NA | NA |
| ZNF137P | LGG | 510 | 1.4 | 1.00E−04 | 0.000556 | NA | NA | NA | NA | NA |
| ZNF155 | LGG | 510 | 1.4 | 1.00E−04 | 0.000556 | NA | NA | NA | NA | NA |
| ZNF160 | LGG | 510 | 1.3 | 0.0042 | 0.012664 | NA | NA | NA | NA | NA |
| ZNF17 | LGG | 510 | 1.3 | 0.0062 | 0.017493 | NA | NA | NA | NA | NA |
| ZNF175 | LGG | 510 | 1.2 | 0.0129 | 0.033745 | NA | NA | NA | NA | NA |
| ZNF180 | LGG | 510 | 1.5 | 0 | 0 | NA | NA | NA | NA | NA |
| ZNF20 | LGG | 510 | 1.4 | 1.00E−04 | 0.000556 | NA | NA | NA | NA | NA |
| ZNF211 | LGG | 510 | 1.4 | 0 | 0 | NA | NA | NA | NA | NA |
| ZNF214 | LGG | 510 | 1.3 | 0.0134 | 0.03437 | NA | NA | NA | NA | NA |
| ZNF221 | LGG | 510 | 1.2 | 0.0147 | 0.036984 | NA | NA | NA | NA | NA |
| ZNF224 | LGG | 510 | 1.5 | 0 | 0 | NA | NA | NA | NA | NA |
| ZNF227 | LGG | 510 | 1.2 | 0.0124 | 0.032653 | NA | NA | NA | NA | NA |
| ZNF229 | LGG | 510 | 1.3 | 0.0013 | 0.005187 | NA | NA | NA | NA | NA |
| ZNF230 | LGG | 510 | 1.5 | 0 | 0 | NA | NA | NA | NA | NA |
| ZNF235 | LGG | 510 | 1.4 | 1.00E−04 | 0.000556 | NA | NA | NA | NA | NA |
| ZNF248 | LIHC | 370 | 1.4 | 4.00E−04 | 0.0395 | NA | NA | NA | NA | NA |
| ZNF252P | UVM | 80 | 1.8 | 0.005 | 0.02487 | NA | NA | NA | NA | NA |
| ZNF256 | LGG | 510 | 1.2 | 0.0184 | 0.044048 | NA | NA | NA | NA | NA |
| ZNF260 | LGG | 510 | 1.3 | 0.003 | 0.009958 | NA | NA | NA | NA | NA |
| ZNF264 | LGG | 510 | 1.3 | 9.00E−04 | 0.003823 | NA | NA | NA | NA | NA |
| ZNF266 | KIRC | 529 | 1.3 | 9.00E−04 | 0.002539 | NA | NA | NA | NA | NA |
| ZNF268 | LGG | 510 | 1.3 | 0.002 | 0.007596 | NA | NA | NA | NA | NA |
| ZNF274 | PRAD | 496 | 2.8 | 0 | 0 | NA | NA | NA | NA | NA |
| ZNF28 | LGG | 510 | 1.6 | 0 | 0 | NA | NA | NA | NA | NA |
| ZNF300 | KIRC | 529 | 1.3 | 3.00E−04 | 0.001004 | NA | NA | NA | NA | NA |
| ZNF304 | LGG | 510 | 1.3 | 0.0039 | 0.011942 | NA | NA | NA | NA | NA |
| ZNF316 | CESC | 304 | 1.6 | 0 | 0 | NA | NA | NA | NA | NA |
| ZNF320 | LGG | 510 | 1.4 | 3.00E−04 | 0.0015 | NA | NA | NA | NA | NA |
| ZNF331 | LGG | 510 | 1.2 | 0.0202 | 0.047778 | NA | NA | NA | NA | NA |
| ZNF333 | KIRC | 529 | 1.2 | 0.0136 | 0.026726 | NA | NA | NA | NA | NA |
| ZNF334 | KIRC | 529 | 1.3 | 0.0012 | 0.003203 | NA | NA | NA | NA | NA |
| ZNF345 | LGG | 510 | 1.4 | 0 | 0 | NA | NA | NA | NA | NA |
| ZNF347 | LGG | 510 | 1.3 | 0 | 0 | NA | NA | NA | NA | NA |
| ZNF35 | LGG | 510 | 1.4 | 8.00E−04 | 0.003435 | NA | NA | NA | NA | NA |
| ZNF350 | LGG | 510 | 1.3 | 0.0036 | 0.011376 | NA | NA | NA | NA | NA |
| ZNF354B | KIRC | 529 | 1.2 | 0.0138 | 0.026985 | NA | NA | NA | NA | NA |
| ZNF37BP | KIRC | 529 | 1.3 | 4.00E−04 | 0.001274 | NA | NA | NA | NA | NA |
| ZNF383 | LGG | 510 | 1.3 | 0.001 | 0.004158 | NA | NA | NA | NA | NA |
| ZNF404 | KIRC | 529 | 1.3 | 0 | 0 | NA | NA | NA | NA | NA |
| ZNF416 | LGG | 510 | 1.4 | 1.00E−04 | 0.000556 | NA | NA | NA | NA | NA |
| ZNF417 | LGG | 510 | 1.4 | 3.00E−04 | 0.0015 | NA | NA | NA | NA | NA |
| ZNF418 | PRAD | 496 | 2.1 | 6.00E−04 | 0.0395 | NA | NA | NA | NA | NA |
| ZNF436 | LGG | 510 | 1.4 | 0 | 0 | NA | NA | NA | NA | NA |
| ZNF439 | PCPG | 178 | 2.8 | 0 | 0 | NA | NA | NA | NA | NA |
| ZNF44 | LGG | 510 | 1.3 | 0.0018 | 0.006971 | NA | NA | NA | NA | NA |
| ZNF440 | PCPG | 178 | 2.5 | 3.00E−04 | 0.0395 | NA | NA | NA | NA | NA |
| ZNF443 | LGG | 510 | 1.5 | 0 | 0 | NA | NA | NA | NA | NA |
| ZNF446 | KIRC | 529 | 1.3 | 0.0012 | 0.003203 | NA | NA | NA | NA | NA |
| ZNF45 | LGG | 510 | 1.6 | 0 | 0 | NA | NA | NA | NA | NA |
| ZNF460 | LGG | 510 | 1.2 | 0.0206 | 0.048435 | NA | NA | NA | NA | NA |
| ZNF461 | LGG | 510 | 1.4 | 4.00E−04 | 0.001881 | NA | NA | NA | NA | NA |
| ZNF468 | LGG | 510 | 1.2 | 0.0058 | 0.016723 | NA | NA | NA | NA | NA |
| ZNF470 | LGG | 510 | 1.6 | 0 | 0 | NA | NA | NA | NA | NA |
| ZNF471 | LGG | 510 | 1.2 | 0.0148 | 0.037 | NA | NA | NA | NA | NA |
| ZNF486 | LGG | 510 | 1.3 | 0.0028 | 0.009788 | NA | NA | NA | NA | NA |
| ZNF487 | KIRC | 529 | 1.3 | 1.00E−04 | 0.000387 | NA | NA | NA | NA | NA |
| ZNF497 | LGG | 510 | 1.3 | 0.0031 | 0.010037 | NA | NA | NA | NA | NA |
| ZNF527 | LGG | 510 | 1.3 | 0.001 | 0.004158 | NA | NA | NA | NA | NA |
| ZNF528 | LGG | 510 | 1.5 | 0 | 0 | NA | NA | NA | NA | NA |
| ZNF543 | LGG | 510 | 1.3 | 0.0083 | 0.022455 | NA | NA | NA | NA | NA |
| ZNF544 | LGG | 510 | 1.4 | 1.00E−04 | 0.000556 | NA | NA | NA | NA | NA |
| ZNF548 | LGG | 510 | 1.3 | 6.00E−04 | 0.002633 | NA | NA | NA | NA | NA |
| ZNF549 | LGG | 510 | 1.4 | 5.00E−04 | 0.00227 | NA | NA | NA | NA | NA |
| ZNF552 | LGG | 510 | 1.6 | 0 | 0 | NA | NA | NA | NA | NA |
| ZNF557 | LGG | 510 | 1.2 | 0.0174 | 0.042426 | NA | NA | NA | NA | NA |
| ZNF561 | LGG | 510 | 1.2 | 0.0134 | 0.03437 | NA | NA | NA | NA | NA |
| ZNF562 | KICH | 64 | 3.6 | 4.00E−04 | 0.0395 | NA | NA | NA | NA | NA |
| ZNF566 | LGG | 510 | 1.3 | 0.0031 | 0.010037 | NA | NA | NA | NA | NA |
| ZNF567 | LGG | 510 | 1.4 | 2.00E−04 | 0.001039 | NA | NA | NA | NA | NA |
| ZNF568 | LGG | 510 | 1.4 | 0 | 0 | NA | NA | NA | NA | NA |
| ZNF569 | LGG | 510 | 1.2 | 0.0163 | 0.039991 | NA | NA | NA | NA | NA |
| ZNF57 | PRAD | 496 | 3.5 | 2.00E−04 | 0.026333 | NA | NA | NA | NA | NA |
| ZNF570 | LGG | 510 | 1.3 | 0.003 | 0.009958 | NA | NA | NA | NA | NA |
| ZNF571 | LGG | 510 | 1.2 | 0.0209 | 0.048562 | NA | NA | NA | NA | NA |
| ZNF573 | LGG | 510 | 1.3 | 0.0038 | 0.011819 | NA | NA | NA | NA | NA |
| ZNF577 | LGG | 510 | 1.2 | 0.0158 | 0.039006 | NA | NA | NA | NA | NA |
| ZNF582 | LGG | 510 | 1.3 | 0.0015 | 0.005925 | NA | NA | NA | NA | NA |
| ZNF583 | LGG | 510 | 1.3 | 0.002 | 0.007596 | NA | NA | NA | NA | NA |
| ZNF584 | LGG | 510 | 1.3 | 0.0033 | 0.010598 | NA | NA | NA | NA | NA |
| ZNF585A | LGG | 510 | 1.3 | 0.0063 | 0.017649 | NA | NA | NA | NA | NA |
| ZNF585B | LGG | 510 | 1.4 | 0 | 0 | NA | NA | NA | NA | NA |
| ZNF586 | LGG | 510 | 1.4 | 4.00E−04 | 0.001881 | NA | NA | NA | NA | NA |
| ZNF599 | LGG | 510 | 1.4 | 0 | 0 | NA | NA | NA | NA | NA |
| ZNF605 | KIRC | 529 | 1.2 | 0.0047 | 0.010372 | NA | NA | NA | NA | NA |
| ZNF606 | LGG | 510 | 1.3 | 0.0054 | 0.015684 | NA | NA | NA | NA | NA |
| ZNF607 | LGG | 510 | 1.3 | 0.0051 | 0.014922 | NA | NA | NA | NA | NA |
| ZNF611 | LGG | 510 | 1.3 | 0.0011 | 0.004526 | NA | NA | NA | NA | NA |
| ZNF613 | LGG | 510 | 1.4 | 0 | 0 | NA | NA | NA | NA | NA |
| ZNF615 | LGG | 510 | 1.3 | 0.0062 | 0.017493 | NA | NA | NA | NA | NA |
| ZNF616 | LGG | 510 | 1.2 | 0.019 | 0.045211 | NA | NA | NA | NA | NA |
| ZNF655 | LGG | 510 | 1.5 | 0 | 0 | NA | NA | NA | NA | NA |
| ZNF662 | LGG | 510 | 1.4 | 1.00E−04 | 0.000556 | NA | NA | NA | NA | NA |
| ZNF665 | LGG | 510 | 1.3 | 0.0042 | 0.012664 | NA | NA | NA | NA | NA |
| ZNF680 | UVM | 80 | 1.8 | 0.0082 | 0.036393 | NA | NA | NA | NA | NA |
| ZNF682 | KIRC | 529 | 1.3 | 1.00E−04 | 0.000387 | NA | NA | NA | NA | NA |
| ZNF684 | LGG | 510 | 1.4 | 4.00E−04 | 0.001881 | NA | NA | NA | NA | NA |
| ZNF69 | LGG | 510 | 1.6 | 0 | 0 | NA | NA | NA | NA | NA |
| ZNF701 | LGG | 510 | 1.7 | 0 | 0 | NA | NA | NA | NA | NA |
| ZNF713 | LGG | 510 | 1.2 | 0.0093 | 0.024821 | NA | NA | NA | NA | NA |
| ZNF732 | KIRC | 529 | 1.2 | 0.0101 | 0.020671 | NA | NA | NA | NA | NA |
| ZNF737 | MESO | 85 | −2 | 0 | 0 | NA | NA | NA | NA | NA |
| ZNF75A | LGG | 510 | 1.3 | 0.0028 | 0.009788 | NA | NA | NA | NA | NA |
| ZNF761 | LGG | 510 | 1.6 | 0 | 0 | NA | NA | NA | NA | NA |
| ZNF765 | LGG | 510 | 1.6 | 0 | 0 | NA | NA | NA | NA | NA |
| ZNF77 | PCPG | 178 | 2.8 | 1.00E−04 | 0.01975 | NA | NA | NA | NA | NA |
| ZNF773 | LGG | 510 | 1.4 | 0 | 0 | NA | NA | NA | NA | NA |
| ZNF776 | LGG | 510 | 1.5 | 0 | 0 | NA | NA | NA | NA | NA |
| ZNF788 | LGG | 510 | 1.3 | 6.00E−04 | 0.002633 | NA | NA | NA | NA | NA |
| ZNF79 | PRAD | 496 | 2 | 2.00E−04 | 0.026333 | NA | NA | NA | NA | NA |
| ZNF790 | LGG | 510 | 1.4 | 0 | 0 | NA | NA | NA | NA | NA |
| ZNF799 | LGG | 510 | 1.5 | 0 | 0 | NA | NA | NA | NA | NA |
| ZNF805 | LGG | 510 | 1.5 | 0 | 0 | NA | NA | NA | NA | NA |
| ZNF808 | LGG | 510 | 1.4 | 3.00E−04 | 0.0015 | NA | NA | NA | NA | NA |
| ZNF816 | LGG | 510 | 1.9 | 0 | 0 | NA | NA | NA | NA | NA |
| ZNF829 | LGG | 510 | 1.3 | 1.00E−04 | 0.000556 | NA | NA | NA | NA | NA |
| ZNF837 | LGG | 510 | 1.5 | 0 | 0 | NA | NA | NA | NA | NA |
| ZNF845 | LGG | 510 | 1.3 | 1.00E−04 | 0.000556 | NA | NA | NA | NA | NA |
| ZNF860 | LGG | 510 | 1.2 | 0.0066 | 0.018104 | NA | NA | NA | NA | NA |
| ZNF880 | LGG | 510 | 1.2 | 0.014 | 0.035449 | NA | NA | NA | NA | NA |
| ZNF90 | LGG | 510 | 1.4 | 0 | 0 | NA | NA | NA | NA | NA |
| ENSG00000267500 | LGG | 510 | 1.3 | 6.00E−04 | 0.002633 | NA | NA | NA | NA | NA |
| ENSG00000267419 | LGG | 510 | 1.3 | 0.0022 | 0.008046 | NA | NA | NA | NA | NA |
| ENSG00000213976 | LGG | 510 | 1.2 | 0.014 | 0.035449 | NA | NA | NA | NA | NA |
| ENSG00000268225 | LGG | 510 | 1.2 | 0.0081 | 0.022066 | NA | NA | NA | NA | NA |
| ENSG00000248452 | KIRP | 287 | 1.4 | 6.00E−04 | 0.013941 | NA | NA | NA | NA | NA |
| ENSG00000215146 | KIRP | 287 | 1.5 | 0.0019 | 0.03263 | NA | NA | NA | NA | NA |
| Name | cancer | n | HR | Pvalue | FDR | cancer | n | HR | Pvalue | FDR |
| ZNF695 | SARC | 259 | 1.4 | 1.00E−04 | 0.01975 | MESO | 85 | 1.68 | 2.00E−04 | 0.0060769 |
| ZNF558 | KIRC | 529 | 1.19 | 0.0039 | 0.0088029 | NA | NA | NA | NA | NA |
| ZNF239 | KIRC | 529 | 1.17 | 0.0228 | 0.0413119 | NA | NA | NA | NA | NA |
| ZNF700 | PRAD | 496 | 2.42 | 3.00E−04 | 0.029625 | NA | NA | NA | NA | NA |
| ZNF587 | SARC | 259 | 1.45 | 3.00E−04 | 0.0395 | NA | NA | NA | NA | NA |
| ZNF7 | UVM | 80 | 1.66 | 0.0115 | 0.0478158 | NA | NA | NA | NA | NA |
| ZNF783 | NA | NA | NA | NA | NA | NA | NA | NA | NA | NA |
| ZNF107 | NA | NA | NA | NA | NA | NA | NA | NA | NA | NA |
| ZNF547 | NA | NA | NA | NA | NA | NA | NA | NA | NA | NA |
| ZNF789 | NA | NA | NA | NA | NA | NA | NA | NA | NA | NA |
| ZNF841 | NA | NA | NA | NA | NA | NA | NA | NA | NA | NA |
| ZNF473 | NA | NA | NA | NA | NA | NA | NA | NA | NA | NA |
| ZNF749 | NA | NA | NA | NA | NA | NA | NA | NA | NA | NA |
| RBAK | NA | NA | NA | NA | NA | NA | NA | NA | NA | NA |
| ZFP30 | NA | NA | NA | NA | NA | NA | NA | NA | NA | NA |
| ZFP69B | NA | NA | NA | NA | NA | NA | NA | NA | NA | NA |
| ZNF121 | NA | NA | NA | NA | NA | NA | NA | NA | NA | NA |
| ZNF200 | NA | NA | NA | NA | NA | NA | NA | NA | NA | NA |
| ZNF251 | NA | NA | NA | NA | NA | NA | NA | NA | NA | NA |
| ZNF282 | NA | NA | NA | NA | NA | NA | NA | NA | NA | NA |
| ZNF283 | NA | NA | NA | NA | NA | NA | NA | NA | NA | NA |
| ZNF337 | NA | NA | NA | NA | NA | NA | NA | NA | NA | NA |
| ZNF354A | NA | NA | NA | NA | NA | NA | NA | NA | NA | NA |
| ZNF419 | NA | NA | NA | NA | NA | NA | NA | NA | NA | NA |
| ZNF432 | NA | NA | NA | NA | NA | NA | NA | NA | NA | NA |
| ZNF485 | NA | NA | NA | NA | NA | NA | NA | NA | NA | NA |
| ZNF496 | NA | NA | NA | NA | NA | NA | NA | NA | NA | NA |
| ZNF514 | NA | NA | NA | NA | NA | NA | NA | NA | NA | NA |
| ZNP517 | NA | NA | NA | NA | NA | NA | NA | NA | NA | NA |
| ZNF525 | NA | NA | NA | NA | NA | NA | NA | NA | NA | NA |
| ZNF530 | NA | NA | NA | NA | NA | NA | NA | NA | NA | NA |
| ZNP550 | NA | NA | NA | NA | NA | NA | NA | NA | NA | NA |
| ZNF707 | NA | NA | NA | NA | NA | NA | NA | NA | NA | NA |
| ZNF746 | NA | NA | NA | NA | NA | NA | NA | NA | NA | NA |
| ZNF766 | NA | NA | NA | NA | NA | NA | NA | NA | NA | NA |
| ZNF80 | NA | NA | NA | NA | NA | NA | NA | NA | NA | NA |
| ZNF814 | NA | NA | NA | NA | NA | NA | NA | NA | NA | NA |
| ZNF850 | NA | NA | NA | NA | NA | NA | NA | NA | NA | NA |
| ZSCAN22 | NA | NA | NA | NA | NA | NA | NA | NA | NA | NA |
| ZNF93 | KIRC | 529 | 1.24 | 0.0049 | 0.0107528 | ACC | 79 | 1.68 | 0.002 | 0.0415789 |
| ZNF18 | UVM | 80 | 2.18 | 1.00E−04 | 0.0010676 | NA | NA | NA | NA | NA |
| ZNF202 | NA | NA | NA | NA | NA | NA | NA | NA | NA | NA |
| ZNF117 | NA | NA | NA | NA | NA | NA | NA | NA | NA | NA |
| ZNF600 | NA | NA | NA | NA | NA | NA | NA | NA | NA | NA |
| ZNF83 | NA | NA | NA | NA | NA | NA | NA | NA | NA | NA |
| ZNF71 | NA | NA | NA | NA | NA | NA | NA | NA | NA | NA |
| ZNF101 | NA | NA | NA | NA | NA | NA | NA | NA | NA | NA |
| ZNF267 | NA | NA | NA | NA | NA | NA | NA | NA | NA | NA |
| ZNF480 | NA | NA | NA | NA | NA | NA | NA | NA | NA | NA |
| HKR1 | NA | NA | NA | NA | NA | NA | NA | NA | NA | NA |
| ZFP28 | NA | NA | NA | NA | NA | NA | NA | NA | NA | NA |
| ZFP69 | NA | NA | NA | NA | NA | NA | NA | NA | NA | NA |
| ZIK1 | NA | NA | NA | NA | NA | NA | NA | NA | NA | NA |
| ZKSCAN5 | NA | NA | NA | NA | NA | NA | NA | NA | NA | NA |
| ZNF112 | NA | NA | NA | NA | NA | NA | NA | NA | NA | NA |
| ZNF12 | NA | NA | NA | NA | NA | NA | NA | NA | NA | NA |
| ZNF132 | NA | NA | NA | NA | NA | NA | NA | NA | NA | NA |
| ZNF133 | NA | NA | NA | NA | NA | NA | NA | NA | NA | NA |
| ZNF134 | NA | NA | NA | NA | NA | NA | NA | NA | NA | NA |
| ZNF136 | NA | NA | NA | NA | NA | NA | NA | NA | NA | NA |
| ZNF137P | NA | NA | NA | NA | NA | NA | NA | NA | NA | NA |
| ZNF155 | NA | NA | NA | NA | NA | NA | NA | NA | NA | NA |
| ZNF160 | NA | NA | NA | NA | NA | NA | NA | NA | NA | NA |
| ZNF17 | NA | NA | NA | NA | NA | NA | NA | NA | NA | NA |
| ZNF175 | NA | NA | NA | NA | NA | NA | NA | NA | NA | MA |
| ZNF180 | NA | NA | NA | NA | NA | NA | NA | NA | NA | NA |
| ZNF20 | NA | NA | NA | NA | NA | NA | NA | NA | NA | NA |
| ZNF211 | NA | NA | NA | NA | NA | NA | NA | NA | NA | NA |
| ZNF214 | NA | NA | NA | NA | NA | NA | NA | NA | NA | NA |
| ZNF221 | NA | NA | NA | NA | NA | NA | NA | NA | NA | NA |
| ZNF224 | NA | NA | NA | NA | NA | NA | NA | NA | NA | NA |
| ZNF227 | NA | NA | NA | NA | NA | NA | NA | NA | NA | NA |
| ZNF229 | NA | NA | NA | NA | NA | NA | NA | NA | NA | NA |
| ZNF230 | NA | NA | NA | NA | NA | NA | NA | NA | NA | NA |
| ZNF235 | NA | NA | NA | NA | NA | NA | NA | NA | NA | NA |
| ZNF248 | NA | NA | NA | NA | NA | NA | NA | NA | NA | NA |
| ZNF252P | NA | NA | NA | NA | NA | NA | NA | NA | NA | NA |
| ZNF256 | NA | NA | NA | NA | NA | NA | NA | NA | NA | NA |
| ZNF260 | NA | NA | NA | NA | NA | NA | NA | NA | NA | NA |
| ZNF264 | NA | NA | NA | NA | NA | NA | NA | NA | NA | NA |
| ZNF266 | NA | NA | NA | NA | NA | NA | NA | NA | NA | NA |
| ZNF268 | NA | NA | NA | NA | NA | NA | NA | NA | NA | NA |
| ZNF274 | NA | NA | NA | NA | NA | NA | NA | NA | NA | NA |
| ZNF28 | NA | NA | NA | NA | NA | NA | NA | NA | NA | NA |
| ZNF300 | NA | NA | NA | NA | NA | NA | NA | NA | NA | NA |
| ZNF304 | NA | NA | NA | NA | NA | NA | NA | NA | NA | NA |
| ZNF316 | NA | NA | NA | NA | NA | NA | NA | NA | NA | NA |
| ZNF320 | NA | NA | NA | NA | NA | NA | NA | NA | NA | NA |
| ZNF331 | NA | NA | NA | NA | NA | NA | NA | NA | NA | NA |
| ZNF333 | NA | NA | NA | NA | NA | NA | NA | NA | NA | NA |
| ZNF334 | NA | NA | NA | NA | NA | NA | NA | NA | NA | NA |
| ZNF345 | NA | NA | NA | NA | NA | NA | NA | NA | NA | NA |
| ZNF347 | NA | NA | NA | NA | NA | NA | NA | NA | NA | NA |
| ZNF35 | NA | NA | NA | NA | NA | NA | NA | NA | NA | NA |
| ZNF350 | NA | NA | NA | NA | NA | NA | NA | NA | NA | NA |
| ZNF354B | NA | NA | NA | NA | NA | NA | NA | NA | NA | NA |
| ZNF37BP | NA | NA | NA | NA | NA | NA | NA | NA | NA | NA |
| ZNF383 | NA | NA | NA | NA | NA | NA | NA | NA | NA | NA |
| ZNF404 | NA | NA | NA | NA | NA | NA | NA | NA | NA | NA |
| ZNF416 | NA | NA | NA | NA | NA | NA | NA | NA | NA | NA |
| ZNF417 | NA | NA | NA | NA | NA | NA | NA | NA | NA | NA |
| ZNF418 | NA | NA | NA | NA | NA | NA | NA | NA | NA | NA |
| ZNF436 | NA | NA | NA | NA | NA | NA | NA | NA | NA | NA |
| ZNF439 | NA | NA | NA | NA | NA | NA | NA | NA | NA | NA |
| ZNF44 | NA | NA | NA | NA | NA | NA | NA | NA | NA | NA |
| ZNF440 | NA | NA | NA | NA | NA | NA | NA | NA | NA | NA |
| ZNF443 | NA | NA | NA | NA | NA | NA | NA | NA | NA | NA |
| ZNF446 | NA | NA | NA | NA | NA | NA | NA | NA | NA | NA |
| ZNF45 | NA | NA | NA | NA | NA | NA | NA | NA | NA | NA |
| ZNF460 | NA | NA | NA | NA | NA | NA | NA | NA | NA | NA |
| ZNF461 | NA | NA | NA | NA | NA | NA | NA | NA | NA | NA |
| ZNF468 | NA | NA | NA | NA | NA | NA | NA | NA | NA | NA |
| ZNF470 | NA | NA | NA | NA | NA | NA | NA | NA | NA | NA |
| ZNF471 | NA | NA | NA | NA | NA | NA | NA | NA | NA | NA |
| ZNF486 | NA | NA | NA | NA | NA | NA | NA | NA | NA | NA |
| ZNF487 | NA | NA | NA | NA | NA | NA | NA | NA | NA | NA |
| ZNF497 | NA | NA | NA | NA | NA | NA | NA | NA | NA | NA |
| ZNF527 | NA | NA | NA | NA | NA | NA | NA | NA | NA | NA |
| ZNF528 | NA | NA | NA | NA | NA | NA | NA | NA | NA | NA |
| ZNF543 | NA | NA | NA | NA | NA | NA | NA | NA | NA | NA |
| ZNF544 | NA | NA | NA | NA | NA | NA | NA | NA | NA | NA |
| ZNF548 | NA | NA | NA | NA | NA | NA | NA | NA | NA | NA |
| ZNF549 | NA | NA | NA | NA | NA | NA | NA | NA | NA | NA |
| ZNF552 | NA | NA | NA | NA | NA | NA | NA | NA | NA | NA |
| ZNF557 | NA | NA | NA | NA | NA | NA | NA | NA | NA | NA |
| ZNF561 | NA | NA | NA | NA | NA | NA | NA | NA | NA | NA |
| ZNF562 | NA | NA | NA | NA | NA | NA | NA | NA | NA | NA |
| ZNF566 | NA | NA | NA | NA | NA | NA | NA | NA | NA | NA |
| ZNF567 | NA | NA | NA | NA | NA | NA | NA | NA | NA | NA |
| ZNF568 | NA | NA | NA | NA | NA | NA | NA | NA | NA | NA |
| ZNF569 | NA | NA | NA | NA | NA | NA | NA | NA | NA | NA |
| ZNF57 | NA | NA | NA | NA | NA | NA | NA | NA | NA | NA |
| ZNF570 | NA | NA | NA | NA | NA | NA | NA | NA | NA | NA |
| ZNF571 | NA | NA | NA | NA | NA | NA | NA | NA | NA | NA |
| ZNF573 | NA | NA | NA | NA | NA | NA | NA | NA | NA | NA |
| ZNF577 | NA | NA | NA | NA | NA | NA | NA | NA | NA | NA |
| ZNF582 | NA | NA | NA | NA | NA | NA | NA | NA | NA | NA |
| ZNF583 | NA | NA | NA | NA | NA | NA | NA | NA | NA | NA |
| ZNF584 | NA | NA | NA | NA | NA | NA | NA | NA | NA | NA |
| ZNF585A | NA | NA | NA | NA | NA | NA | NA | NA | NA | NA |
| ZNF585B | NA | NA | NA | NA | NA | NA | NA | NA | NA | NA |
| ZNF586 | NA | NA | NA | NA | NA | NA | NA | NA | NA | NA |
| ZNF599 | NA | NA | NA | NA | NA | NA | NA | NA | NA | NA |
| ZNF605 | NA | NA | NA | NA | NA | NA | NA | NA | NA | NA |
| ZNF606 | NA | NA | NA | NA | NA | NA | NA | NA | NA | NA |
| ZNF607 | NA | NA | NA | NA | NA | NA | NA | NA | NA | NA |
| ZNF611 | NA | NA | NA | NA | NA | NA | NA | NA | NA | NA |
| ZNF613 | NA | NA | NA | NA | NA | NA | NA | NA | NA | NA |
| ZNF615 | NA | NA | NA | NA | NA | NA | NA | NA | NA | NA |
| ZNF616 | NA | NA | NA | NA | NA | NA | NA | NA | NA | NA |
| ZNF655 | NA | NA | NA | NA | NA | NA | NA | NA | NA | NA |
| ZNF662 | NA | NA | NA | NA | NA | NA | NA | NA | NA | NA |
| ZNF665 | NA | NA | NA | NA | NA | NA | NA | NA | NA | NA |
| ZNF680 | NA | NA | NA | NA | NA | NA | NA | NA | NA | NA |
| ZNF682 | NA | NA | NA | NA | NA | NA | NA | NA | NA | NA |
| ZNF684 | NA | NA | NA | NA | NA | NA | NA | NA | NA | NA |
| ZNF69 | NA | NA | NA | NA | NA | NA | NA | NA | NA | NA |
| ZNF701 | NA | NA | NA | NA | NA | NA | NA | NA | NA | NA |
| ZNF713 | NA | NA | NA | NA | NA | NA | NA | NA | NA | NA |
| ZNF732 | NA | NA | NA | NA | NA | NA | NA | NA | NA | NA |
| ZNF737 | NA | NA | NA | NA | NA | NA | NA | NA | NA | NA |
| ZNF75A | NA | NA | NA | NA | NA | NA | NA | NA | NA | NA |
| ZNF761 | NA | NA | NA | NA | NA | NA | NA | NA | NA | NA |
| ZNF765 | NA | NA | NA | NA | NA | NA | NA | NA | NA | NA |
| ZNF77 | NA | NA | NA | NA | NA | NA | NA | NA | NA | NA |
| ZNF773 | NA | NA | NA | NA | NA | NA | NA | NA | NA | NA |
| ZNF776 | NA | NA | NA | NA | NA | NA | NA | NA | NA | NA |
| ZNF788 | NA | NA | NA | NA | NA | NA | NA | NA | NA | NA |
| ZNF79 | NA | NA | NA | NA | NA | NA | NA | NA | NA | NA |
| ZNF790 | NA | NA | NA | NA | NA | NA | NA | NA | NA | NA |
| ZNF799 | NA | NA | NA | NA | NA | NA | NA | NA | NA | NA |
| ZNF805 | NA | NA | NA | NA | NA | NA | NA | NA | NA | NA |
| ZNF808 | NA | NA | NA | NA | NA | NA | NA | NA | NA | NA |
| ZNF816 | NA | NA | NA | NA | NA | NA | NA | NA | NA | NA |
| ZNF829 | NA | NA | NA | NA | NA | NA | NA | NA | NA | NA |
| ZNF837 | NA | NA | NA | NA | NA | NA | NA | NA | NA | NA |
| ZNF845 | NA | NA | NA | NA | NA | NA | NA | NA | NA | NA |
| ZNF860 | NA | NA | NA | NA | NA | NA | NA | NA | NA | NA |
| ZNF880 | NA | NA | NA | NA | NA | NA | NA | NA | NA | NA |
| ZNF90 | NA | NA | NA | NA | NA | NA | NA | NA | NA | NA |
| ENSG00000267500 | NA | NA | NA | NA | NA | NA | NA | NA | NA | NA |
| ENSG00000267419 | NA | NA | NA | NA | NA | NA | NA | NA | NA | NA |
| ENSG00000213976 | NA | NA | NA | NA | NA | NA | NA | NA | NA | NA |
| ENSG00000268225 | NA | NA | NA | NA | NA | NA | NA | NA | NA | NA |
| ENSG00000248452 | NA | NA | NA | NA | NA | NA | NA | NA | NA | NA |
| ENSG00000215146 | NA | NA | NA | NA | NA | NA | NA | NA | NA | NA |

| Name | cancer | n | HR | Pvalue | FDR | Status |
|---|---|---|---|---|---|---|
| ZNF695 | ACC | 79 | 2.31 | 0 | 0 | UP |
| ZNF558 | NA | NA | NA | NA | NA | UP |
| ZNF239 | NA | NA | NA | NA | NA | UP |
| ZNF700 | NA | NA | NA | NA | NA | UP |
| ZNF587 | NA | NA | NA | NA | NA | UP |
| ZNF7 | NA | NA | NA | NA | NA | UP |
| ZNF783 | NA | NA | NA | NA | NA | UP |
| ZNF107 | NA | NA | NA | NA | NA | UP |
| ZNF547 | NA | NA | NA | NA | NA | UP |
| ZNF789 | NA | NA | NA | NA | NA | UP |
| ZNF841 | NA | NA | NA | NA | NA | UP |
| ZNF473 | NA | NA | NA | NA | NA | UP |
| ZNF749 | NA | NA | NA | NA | NA | UP |
| RBAK | NA | NA | NA | NA | NA | UP |
| ZFP30 | NA | NA | NA | NA | NA | UP |
| ZFP69B | NA | NA | NA | NA | NA | UP |
| ZNF121 | NA | NA | NA | NA | NA | UP |
| ZNF200 | NA | NA | NA | NA | NA | UP |
| ZNF251 | NA | NA | NA | NA | NA | UP |
| ZNF282 | NA | NA | NA | NA | NA | UP |
| ZNF283 | NA | NA | NA | NA | NA | UP |
| ZNF337 | NA | NA | NA | NA | NA | UP |
| ZNF354A | NA | NA | NA | NA | NA | UP |
| ZNF419 | NA | NA | NA | NA | NA | UP |
| ZNF432 | NA | NA | NA | NA | NA | UP |
| ZNF485 | NA | NA | NA | NA | NA | UP |
| ZNF496 | NA | NA | NA | NA | NA | UP |
| ZNF514 | NA | NA | NA | NA | NA | UP |
| ZNP517 | NA | NA | NA | NA | NA | UP |
| ZNF525 | NA | NA | NA | NA | NA | UP |
| ZNF530 | NA | NA | NA | NA | NA | UP |
| ZNP550 | NA | NA | NA | NA | NA | UP |
| ZNF707 | NA | NA | NA | NA | NA | UP |
| ZNF746 | NA | NA | NA | NA | NA | UP |
| ZNF766 | NA | NA | NA | NA | NA | UP |
| ZNF80 | NA | NA | NA | NA | NA | UP |
| ZNF814 | NA | NA | NA | NA | NA | UP |
| ZNF850 | NA | NA | NA | NA | NA | UP |
| ZSCAN22 | NA | NA | NA | NA | NA | UP |
| ZNF93 | NA | NA | NA | NA | NA | NA |
| ZNF18 | NA | NA | NA | NA | NA | NA |
| ZNF202 | NA | NA | NA | NA | NA | NA |
| ZNF117 | NA | NA | NA | NA | NA | NA |
| ZNF600 | NA | NA | NA | NA | NA | NA |
| ZNF83 | NA | NA | NA | NA | NA | NA |
| ZNF71 | NA | NA | NA | NA | NA | NA |
| ZNF101 | NA | NA | NA | NA | NA | NA |
| ZNF267 | NA | NA | NA | NA | NA | NA |
| ZNF480 | NA | NA | NA | NA | NA | NA |
| HKR1 | NA | NA | NA | NA | NA | NA |
| ZFP28 | NA | NA | NA | NA | NA | NA |
| ZFP69 | NA | NA | NA | NA | NA | NA |
| ZIK1 | NA | NA | NA | NA | NA | NA |
| ZKSCAN5 | NA | NA | NA | NA | NA | NA |
| ZNF112 | NA | NA | NA | NA | NA | NA |
| ZNF12 | NA | NA | NA | NA | NA | NA |
| ZNF132 | NA | NA | NA | NA | NA | NA |
| ZNF133 | NA | NA | NA | NA | NA | NA |
| ZNF134 | NA | NA | NA | NA | NA | NA |
| ZNF136 | NA | NA | NA | NA | NA | NA |
| ZNF137P | NA | NA | NA | NA | NA | NA |
| ZNF155 | NA | NA | NA | NA | NA | NA |
| ZNF160 | NA | NA | NA | NA | NA | NA |
| ZNF17 | NA | NA | NA | NA | NA | NA |
| ZNF175 | NA | NA | NA | NA | NA | NA |
| ZNF180 | NA | NA | NA | NA | NA | NA |
| ZNF20 | NA | NA | NA | NA | NA | NA |
| ZNF211 | NA | NA | NA | NA | NA | NA |
| ZNF214 | NA | NA | NA | NA | NA | NA |
| ZNF221 | NA | NA | NA | NA | NA | NA |
| ZNF224 | NA | NA | NA | NA | NA | NA |
| ZNF227 | NA | NA | NA | NA | NA | NA |
| ZNF229 | NA | NA | NA | NA | NA | NA |
| ZNF230 | NA | NA | NA | NA | NA | NA |
| ZNF235 | NA | NA | NA | NA | NA | NA |
| ZNF248 | NA | NA | NA | NA | NA | NA |
| ZNF252P | NA | NA | NA | NA | NA | NA |
| ZNF256 | NA | NA | NA | NA | NA | NA |
| ZNF260 | NA | NA | NA | NA | NA | NA |
| ZNF264 | NA | NA | NA | NA | NA | NA |
| ZNF266 | NA | NA | NA | NA | NA | NA |
| ZNF268 | NA | NA | NA | NA | NA | NA |
| ZNF274 | NA | NA | NA | NA | NA | NA |
| ZNF28 | NA | NA | NA | NA | NA | NA |
| ZNF300 | NA | NA | NA | NA | NA | NA |
| ZNF304 | NA | NA | NA | NA | NA | NA |
| ZNF316 | NA | NA | NA | NA | NA | NA |
| ZNF320 | NA | NA | NA | NA | NA | NA |
| ZNF331 | NA | NA | NA | NA | NA | NA |
| ZNF333 | NA | NA | NA | NA | NA | NA |
| ZNF334 | NA | NA | NA | NA | NA | NA |
| ZNF345 | NA | NA | NA | NA | NA | NA |
| ZNF347 | NA | NA | NA | NA | NA | NA |
| ZNF35 | NA | NA | NA | NA | NA | NA |
| ZNF350 | NA | NA | NA | NA | NA | NA |
| ZNF3548 | NA | NA | NA | NA | NA | NA |
| ZNF37BP | NA | NA | NA | NA | NA | NA |
| ZNF383 | NA | NA | NA | NA | NA | NA |
| ZNF404 | NA | NA | NA | NA | NA | NA |
| ZNF416 | NA | NA | NA | NA | NA | NA |
| ZNF417 | NA | NA | NA | NA | NA | NA |
| ZNF418 | NA | NA | NA | NA | NA | NA |
| ZNF436 | NA | NA | NA | NA | NA | NA |
| ZNF439 | NA | NA | NA | NA | NA | NA |
| ZNF44 | NA | NA | NA | NA | NA | NA |
| ZNF440 | NA | NA | NA | NA | NA | NA |
| ZNF443 | NA | NA | NA | NA | NA | NA |
| ZNF446 | NA | NA | NA | NA | NA | NA |
| ZNF45 | NA | NA | NA | NA | NA | NA |
| ZNF460 | NA | NA | NA | NA | NA | NA |
| ZNF461 | NA | NA | NA | NA | NA | NA |
| ZNF468 | NA | NA | NA | NA | NA | NA |
| ZNF470 | NA | NA | NA | NA | NA | NA |
| ZNF471 | NA | NA | NA | NA | NA | NA |
| ZNF486 | NA | NA | NA | NA | NA | NA |
| ZNF487 | NA | NA | NA | NA | NA | NA |
| ZNF497 | NA | NA | NA | NA | NA | NA |
| ZNF527 | NA | NA | NA | NA | NA | NA |
| ZNF528 | NA | NA | NA | NA | NA | NA |
| ZNF543 | NA | NA | NA | NA | NA | NA |
| ZNF544 | NA | NA | NA | NA | NA | NA |
| ZNF548 | NA | NA | NA | NA | NA | NA |
| ZNF549 | NA | NA | NA | NA | NA | NA |
| ZNF552 | NA | NA | NA | NA | NA | NA |
| ZNF557 | NA | NA | NA | NA | NA | NA |
| ZNF561 | NA | NA | NA | NA | NA | NA |
| ZNF562 | NA | NA | NA | NA | NA | NA |
| ZNF566 | NA | NA | NA | NA | NA | NA |
| ZNF567 | NA | NA | NA | NA | NA | NA |
| ZNF568 | NA | NA | NA | NA | NA | NA |
| ZNF569 | NA | NA | NA | NA | NA | NA |
| ZNF57 | NA | NA | NA | NA | NA | NA |
| ZNF570 | NA | NA | NA | NA | NA | NA |
| ZNF571 | NA | NA | NA | NA | NA | NA |
| ZNF573 | NA | NA | NA | NA | NA | NA |
| ZNF577 | NA | NA | NA | NA | NA | NA |
| ZNF582 | NA | NA | NA | NA | NA | NA |
| ZNF583 | NA | NA | NA | NA | NA | NA |
| ZNF584 | NA | NA | NA | NA | NA | NA |
| ZNF585A | NA | NA | NA | NA | NA | NA |
| ZNF585B | NA | NA | NA | NA | NA | NA |
| ZNF586 | NA | NA | NA | NA | NA | NA |
| ZNF599 | NA | NA | NA | NA | NA | NA |
| ZNF605 | NA | NA | NA | NA | NA | NA |
| ZNF606 | NA | NA | NA | NA | NA | NA |
| ZNF607 | NA | NA | NA | NA | NA | NA |
| ZNF611 | NA | NA | NA | NA | NA | NA |
| ZNF613 | NA | NA | NA | NA | NA | NA |
| ZNF615 | NA | NA | NA | NA | NA | NA |
| ZNF616 | NA | NA | NA | NA | NA | NA |
| ZNF655 | NA | NA | NA | NA | NA | NA |
| ZNF662 | NA | NA | NA | NA | NA | NA |
| ZNF665 | NA | NA | NA | NA | NA | NA |
| ZNF680 | NA | NA | NA | NA | NA | NA |
| ZNF682 | NA | NA | NA | NA | NA | NA |
| ZNF684 | NA | NA | NA | NA | NA | NA |
| ZNF69 | NA | NA | NA | NA | NA | NA |
| ZNF701 | NA | NA | NA | NA | NA | NA |
| ZNF713 | NA | NA | NA | NA | NA | NA |
| ZNF732 | NA | NA | NA | NA | NA | NA |
| ZNF737 | NA | NA | NA | NA | NA | NA |
| ZNF75A | NA | NA | NA | NA | NA | NA |
| ZNF761 | NA | NA | NA | NA | NA | NA |
| ZNF765 | NA | NA | NA | NA | NA | NA |
| ZNF77 | NA | NA | NA | NA | NA | NA |
| ZNF773 | NA | NA | NA | NA | NA | NA |
| ZNF776 | NA | NA | NA | NA | NA | NA |
| ZNF788 | NA | NA | NA | NA | NA | NA |
| ZNF79 | NA | NA | NA | NA | NA | NA |
| ZNF790 | NA | NA | NA | NA | NA | NA |
| ZNF799 | NA | NA | NA | NA | NA | NA |
| ZNF805 | NA | NA | NA | NA | NA | NA |
| ZNF808 | NA | NA | NA | NA | NA | NA |
| ZNF816 | NA | NA | NA | NA | NA | NA |
| ZNF829 | NA | NA | NA | NA | NA | NA |
| ZNF837 | NA | NA | NA | NA | NA | NA |
| ZNF845 | NA | NA | NA | NA | NA | NA |
| ZNF860 | NA | NA | NA | NA | NA | NA |
| ZNF880 | NA | NA | NA | NA | NA | NA |
| ZNF90 | NA | NA | NA | NA | NA | NA |
| ENSG00000267500 | NA | NA | NA | NA | NA | NA |
| ENSG00000267419 | NA | NA | NA | NA | NA | NA |
| ENSG00000213976 | NA | NA | NA | NA | NA | NA |
| ENSG00000268225 | NA | NA | NA | NA | NA | NA |
| ENSG00000248452 | NA | NA | NA | NA | NA | NA |
| ENSG00000215146 | NA | NA | NA | NA | NA | NA |

ACC Adrenocortical carcinoma
COAD Colon adenocarcinoma
STAD Stomach adenocarcinoma
THCA Thyroid carcinoma
MESO Mesothelioma
UVM Uveal Melanoma
OV Ovarian serous cystadenocarcinoma
TGCT Testicular Germ Cell Tumors
PRAD Prostate adenocarcinoma
BRCA Breast invasive carcinoma
LUSC Lung squamous cell carcinoma
SARC Sarcoma
THYM Thymoma
PAAD Pancreatic adenocarcinoma
UCS Uterine Carcinosarcoma
HNSC Head and Neck squamous cell carcinoma
KIRC Kidney renal clear cell carcinoma
SKCM Skin Cutaneous Melanoma
BLCA Bladder Urothelial Carcinoma
LUAD Lung adenocarcinoma
READ Rectum adenocarcinoma
PCPG Pheochromocytoma and Paraganglioma
KIRP Kidney renal papillary cell carcinoma
ESCA Esophageal carcinoma
LIHC Liver hepatocellular carcinoma
KICH Kidney Chromophobe
UCEC Uterine Corpus Endometrial Carcinoma
CHOL Cholangiocarcinoma
LGG Brain Lower Grade Glioma
CESC Cervical squamous cell carcinoma and endocervical adenocarcinoma
GBM Glioblastoma multiforme
DLBC Lymphoid Neoplasm Diffuse Large B-cell Lymphoma

### REFERENCE LIST

1. Swerdlow S H, Campo E, Pileri S A, et al. The 2016 revision of the World Health Organization classification of lymphoid neoplasms. Blood. 2016; 127(20):2375-2390. doi: 10.1182/blood-2016-01-643569
2. Rosenwald A, Staudt L M. Gene Expression Profiling of Diffuse Large B-Cell Lymphoma. Leukemia & Lymphoma. 2003; 44(sup3): S41-S47. doi: 10.1080/10428190310001623775
3. Clozel T, Yang S, Elstrom R L, et al. Mechanism-based epigenetic chemosensitization therapy of diffuse large B-cell lymphoma. Cancer Discov. 2013; 3(9): 1002-1019. doi:10.1158/2159-8290.CD-13-0117
4. Martin P, Bartlett N L, Chavez J C, et al. Phase 1 study of oral azacitidine (CC-486) plus R-CHOP in previously untreated intermediate- to high-risk DLBCL. Blood. 2022; 139(8): 1147-1159. doi: 10.1182/blood.2021011679
5. Reddy A, Zhang J, Davis N S, et al. Genetic and Functional Drivers of Diffuse Large B Cell Lymphoma. Cell. 2017; 171(2):481-494.e15. doi:10.1016/j.cell.2017.09.027
6. Lenz G, Wright G W, Emre N C T, et al. Molecular subtypes of diffuse large B-cell lymphoma arise by distinct genetic pathways. Proc Natl Acad Sci USA. 2008; 105(36): 13520-13525. doi: 10.1073/pnas.0804295105
7. Dubois S, Tesson B, Mareschal S, et al. Refining diffuse large B-cell lymphoma subgroups using integrated analysis of molecular profiles. EBioMedicine. 2019; 48:58-69. doi: 10.1016/j.ebiom.2019.09.034
8. Zhao M, Kim P, Mitra R, Zhao J, Zhao Z. TSGene 2.0: an updated literature-based knowledgebase for tumor suppressor genes. Nucleic Acids Res. 2016; 44(D1):D1023-D1031. doi:10.1093/nar/gkv1268
9. Rowe H M, Jakobsson J, Mesnard D, et al. KAP1 controls endogenous retroviruses in embryonic stem cells. Nature. 2010; 463(7278):237-240. doi:10.1038/nature08674
10. Castro-Diaz N, Ecco G, Coluccio A, et al. Evolutionally dynamic L1 regulation in embryonic stem cells. Genes Dev. 2014; 28(13): 1397-1409. doi: 10.1101/gad.241661.114
11. Ecco G, Imbeault M, Trono D. KRAB zinc finger proteins. Development. 2017; 144(15):2719-2729. doi: 10.1242/dev.132605
12. Playfoot C J, Duc J, Sheppard S, et al. Transposable elements and their KZFP controllers are drivers of transcriptional innovation in the developing human brain. Genome Res. 2021; 31(9): 1531-1545. doi: 10.1101/gr.275133.120

1. An agent modulating the expression and/or activity of one or more i) KRAB-containing zinc finger protein (KZFP), ii) mRNA encoding a KZFP, and/or iii) KZFP gene, for use in the treatment and/or prevention of cancer and/or cancer metastasis in a subject in need thereof, wherein said one or more KZFP is selected from the group comprising those listed in Table 1, or a combination of one or more thereof.

2. The agent for use of claim 1, wherein said agent inhibits the translation of one or more RNA encoding a KZFP.

3. The agent for use of claim 1, wherein the agent inhibits the transcription of one or more KZFP gene encoding a KZFP.

4. The agent for use of claim 1, wherein the agent modulates the activity of one or more KZFP protein by enhancing and/or favoring the degradation of said one or more KZFP, by preventing the recognition of its targets or by any other mean resulting in a loss of function.

5. The agent for use of any one of the preceding claims, wherein the agent is selected from the group comprising a nucleic acid, a chemical compound, a peptide or analog thereof, an antibody or an antigen-binding fragment thereof, and an antibody mimetic, or a combination of one or more thereof.

6. The agent for use of claim 5, wherein the nucleic acid is selected from the group comprising a nucleic acid encoding an siRNA, an miRNA, a piRNA, an hnRNA, an snRNA, an sg RNA, a CRISPR-based loss-of-function system, an esiRNA, an shRNA, and an antisense oligonucleotide, or a combination of one or more thereof.

7. The agent for use of any one of the preceding claims, wherein the cancer is selected from a hematological cancer (such as e.g. a Non-Hodgkin lymphoma (NHL) or a leukemia), and a solid cancer (such as e.g. a colon cancer).

8. The agent for use of any one of the preceding claims, wherein
the one or more mRNA encoding a KZFP is selected from the group comprising, or represented by, cDNA sequences SEQ ID No. 11, SEQ ID No. 13, SEQ ID No. 63, SEQ ID No. 81, SEQ ID No. 129, SEQ ID No. 155, SEQ ID No. 181, SEQ ID No. 203, SEQ ID No. 209, and SEQ ID No. 231, a fragment or a variant thereof or a combination of one or more thereof and/or wherein,
the one or more KZF protein is selected from the group comprising, or consisting of, SEQ ID No. 12, SEQ ID No. 14, SEQ ID No. 64, SEQ ID No. 82, SEQ ID No. 130, SEQ ID No. 156, SEQ ID No. 182, SEQ ID No. 204, SEQ ID No. 210, and SEQ ID No. 232, a fragment or a variant thereof or a combination of one or more thereof.

9. A plasmid or a vector comprising one or more nucleic acid(s) encoding the siRNA, miRNA, piRNA, hnRNA, snRNA, sg RNA, CRISPR-based loss-of-function system, esiRNA, shRNA, and antisense oligonucleotide, or combination of one or more thereof, of claim 6.

10. A host cell comprising, or modified by the introduction of,
i) a plasmid or vector of claim 9, or
ii) one or more nucleic acid(s) encoding the siRNA, miRNA, piRNA, hnRNA, snRNA, sg RNA, CRISPR-based loss-of-function system, esiRNA, shRNA, and antisense oligonucleotide, or combination of one or more thereof, of claim 6.

11. A pharmaceutical composition comprising
i) a therapeutically effective amount of an agent modulating the expression and/or activity of one or more i) KZFP, ii) mRNA encoding a KZFP and/or iii) KZFP gene, of anyone of claims 1 to 8, or
ii) a plasmid or a vector of claim 9, or
iii) a host cell of claim 10,
and a pharmaceutically acceptable carrier or diluent.

12. The pharmaceutical composition of claim 11, for use in the treatment and/or prevention of cancer and/or cancer metastasis in a subject in need thereof.

13. The pharmaceutical composition of claim 9 or 10, further comprising one or more additional anticancer agent or therapy.

14. The pharmaceutical composition of any one of claims 11 to 13, wherein said KZFP is selected from the group comprising ZNF671, ZNF776, ZNF586, ZNF552, ZNF587B, ZNF417, ZNF107, ZNF121, ZNF200, ZNF239, ZNF473, ZNF547, ZNF558, ZNF587, ZNF695, ZNF7, ZNF700, ZNF749, ZNF766, ZNF783, ZNF789, ZNF814, ZNF841, and ZNF850, or a combination of one or more thereof.

15. The pharmaceutical composition of any one of claims 11 to 14, wherein the cancer is selected from a hematological cancer (such as e.g. a Non-Hodgkin lymphoma (NHL) or a leukemia), and a solid cancer (such as e.g. a colon cancer).

16. The pharmaceutical composition of any one of claims 13 to 15, wherein the additional anticancer agent or therapy is selected from the group comprising radiotherapy, chemotherapy, immunotherapy, inhibitors of DNA methylation, targeted therapy and hormone therapy, or a combination of one of more thereof.

17. A method of diagnosing cancer and/or cancer metastasis in a subject comprising:
(a) detecting, directly or indirectly, and measuring the level of transcription and/or expression and/or activity of one or more KZFP in a sample obtained from said subject;
(b) comparing the level of transcription and/or expression and/or activity of said one or more KZFP to a control biological sample for the same one or more KZFP;
wherein a differential transcription and/or expression and/or activity of one or more one or more KZFP in said biological sample, relative to the level of corresponding said one or more KZFP in a control biological sample of a cancer-free subject, is indicative of the subject having cancer and/or cancer metastasis.

18. The method of claim 17, wherein the differential transcription and/or expression and/or activity level of one or more one or more KZFP corresponds to an upregulated expression of said one or more KZFP.

19. The method of claim 18, wherein the upregulated transcription and/or expression and/or activity of said one or more KZFP in a biological sample corresponds to an increase equal or superior to about 5%, preferably equal or superior to about 20%, more preferably equal or superior to about 40%, most preferably equal or superior to about 60%, more preferably equal or superior to about 500%, even more preferably equal or superior to about 1000%, in particular equal or superior to about 5000% when compared to the level of corresponding said one or more KZFP in a control biological sample of a cancer-free subject.

20. The method of any one of claims 17-19, wherein the biological sample is selected from the group comprising whole blood, serum, plasma, semen, saliva, tears, urine, fecal material, sweat, buccal smears, skin, and cancer cells or a combination of one or more of these biological samples.

21. The method of any one of claims 17-20, wherein the KZFP is selected from the group comprising ZNF671, ZNF776, ZNF586, ZNF552, ZNF587B, ZNF417, ZNF107, ZNF121, ZNF200, ZNF239, ZNF473, ZNF547, ZNF558, ZNF587, ZNF695, ZNF7, ZNF700, ZNF749, ZNF766, ZNF783, ZNF789, ZNF814, ZNF841, and ZNF850, or a combination of one or more thereof.

22. The method of claim 21, wherein the KZFP is selected from the group comprising ZNF121, ZNF200, ZNF473, ZNF547, ZNF587, ZNF7, ZNF749, ZNF766, ZNF814, and ZNF850, or a combination of one or more thereof.

23. The method of any one of claims 17-22, wherein the cancer is selected from a hematological cancer (such as e.g. a Non-Hodgkin lymphoma (NHL) or a leukemia), and a solid cancer (such as e.g. a colon cancer).

24. The method of any one of claims 17-22, further comprising administering an agent modulating the expression and/or activity of one or more i) KRAB-containing zinc finger protein (KZFP), ii) mRNA encoding a KZFP, and/or iii) KZFP gene, of anyone of claims 1 to 8.

25. The method of any one of claims 17-22, further comprising administering a pharmaceutical composition comprising
i) a therapeutically effective amount of an agent modulating the expression and/or activity of one or more i) KZFP, ii) mRNA encoding a KZFP and/or iii) KZFP gene, of anyone of claims 1 to 8, or
ii) a plasmid or a vector of claim 9, or
iii) a host cell of claim 10,
and a pharmaceutically acceptable carrier or diluent.

26. A method of determining the progression or regression of a cancer in a subject suffering therefrom and under cancer treatment, said method comprising:
(a) detecting, directly or indirectly, and measuring the level of transcription and/or expression and/or activity of one or more KZFP in a sample obtained from said subject;
(b) periodically comparing the level of transcription and/or expression and/or activity of said one or more KZFP,
wherein an alteration in the level of transcription and/or expression and/or activity of one or more KZFP in a sample obtained from the same subject, relative to the level of transcription and/or expression and/or activity of one or more KZFP, determined previously, is indicative of the progression or regression of said cancer.

27. The method of claim 26, wherein the alteration in the transcription and/or expression and/or activity level(s) of one or more KZFP corresponds to an enhanced transcription and/or expression and/or activity of said one or more KZFP and is indicative of the progression of said cancer.

28. The method of claim 26 or 27, wherein the alteration in the transcription and/or expression and/or activity level(s) of one or more one or more KZFP corresponds to a decreased transcription and/or expression and/or activity of said one or more KZFP and is indicative of the regression of said cancer.

29. The method of any one of claims 26 to 28, wherein the KZFP is selected from the group comprising ZNF671, ZNF776, ZNF586, ZNF552, ZNF587B, ZNF417, ZNF107, ZNF121, ZNF200, ZNF239, ZNF473, ZNF547, ZNF558, ZNF587, ZNF695, ZNF7, ZNF700, ZNF749, ZNF766, ZNF783, ZNF789, ZNF814, ZNF841, and ZNF850, or a combination of one or more thereof, preferably selected from the group comprising ZNF121, ZNF200, ZNF473, ZNF547, ZNF587, ZNF7, ZNF749, ZNF766, ZNF814, and ZNF850, or a combination of one or more thereof.

30. The method of any one of claims 26 to 29, wherein the cancer is selected from a hematological cancer (such as e.g. a Non-Hodgkin lymphoma (NHL) or a leukemia), and a solid cancer (such as e.g. a colon cancer).

31. The method of any one of claims 26 to 30, further comprising a step of continuing the treatment, adapting the treatment, changing the treatment, or changing the dosage of the treatment.

32. The method of claim 31, wherein the step of changing the treatment comprises i) administering an agent modulating the expression and/or activity of one or more i) KRAB-containing zinc finger protein (KZFP), ii) mRNA encoding a KZFP, and/or iii) KZFP gene, of anyone of claims 1 to 8.

33. A method of treatment of cancer, and/or cancer metastasis comprising administering an agent modulating the expression and/or activity of one or more i) KRAB-containing zinc finger protein (KZFP), ii) mRNA encoding a KZFP, and/or iii) KZFP gene, of anyone of claims 1 to 8 to a subject in need thereof.

34. The method of claim 33, wherein the KZFP is selected from the group comprising ZNF671, ZNF776, ZNF586, ZNF552, ZNF587B, ZNF417, ZNF107, ZNF121, ZNF200, ZNF239, ZNF473, ZNF547, ZNF558, ZNF587, ZNF695, ZNF7, ZNF700, ZNF749, ZNF766, ZNF783, ZNF789, ZNF814, ZNF841, and ZNF850, or a combination of one or more thereof.

35. The method of treatment of claim 33 or 34, wherein said agent is in the form of a pharmaceutical composition according to any of claims 11 to 16.

36. A kit comprising
(a) one or more agents according to any one of claims 1 to 8, plasmid or vector of claim 9, host cell of claim 10 and/or a pharmaceutical composition of any one of claims 11 to 16, and
(b) instructions for use.

37. A method of stratifying a cancer in a subject comprising:
(a) detecting, directly or indirectly, and measuring the level of transcription and/or expression and/or activity of one or more KZFP in a sample obtained from said subject;
(b) comparing the level of transcription and/or expression and/or activity of said one or more KZFP to a control biological sample for the same one or more KZFP;
wherein an alteration in the transcription and/or expression and/or activity level of one or more one or more KZFP in said biological sample, relative to the level of corresponding said one or more KZFP in a control biological sample of a cancer-free subject, is indicative of the disease stage or grade.

38. The method of claim 37, wherein the KZFP is selected from the group comprising ZNF671, ZNF776, ZNF586, ZNF552, ZNF587B, ZNF417, ZNF107, ZNF121, ZNF200, ZNF239, ZNF473, ZNF547, ZNF558, ZNF587, ZNF695, ZNF7, ZNF700, ZNF749, ZNF766, ZNF783, ZNF789, ZNF814, ZNF841, and ZNF850, or a combination of one or more thereof.

39. The pharmaceutical agent of any one of claims 11 to 16, the methods of any one of claims 17 to 35 36 to 38, wherein
the transcript or mRNA encoding a KZFP is selected from the group comprising, or represented by, cDNA sequences: SEQ ID No. 1, SEQ ID No. 3, SEQ ID No. 5, SEQ ID No. 7, SEQ ID No. 9, SEQ ID No. 11, SEQ ID No. 13, and SEQ ID No. 15, a fragment or a variant thereof or a combination of one or more thereof and/or wherein,
the KZF protein is selected from the group comprising, or consisting of, SEQ ID No. 2, SEQ ID No. 4, SEQ ID No. 6, SEQ ID No. 8, SEQ ID No. 10, SEQ ID No. 12, SEQ ID No. 14, and SEQ ID No. 16, a fragment or a variant thereof or a combination of one or more thereof.

40. A method for classifying a cancer subject, the method comprising
(a) detecting, directly or indirectly, and measuring the level of transcription and/or expression and/or activity of one or more KZFP in a biological sample obtained from said subject,
wherein said one or more KZFP is selected from the group listed in Table 1 or a variant or fragment thereof, and
(b) classifying the cancer subject as having a poor prognosis based on the presence and/or alteration of the transcription and/or expression and/or activity level(s) of the one or more KZFP in the biological sample.

41. The method of claim 40, wherein the cancer subject is determined as having a poor prognosis when the transcription and/or expression and/or activity level(s) of one or more KZFP, variant or fragment thereof of Table 1 is upregulated.

42. The method of claim 40 or 41, wherein the cancer subject is determined as having a poor prognosis when an alteration of the transcription and/or expression and/or activity level(s) of one or more KZFP, variant or fragment thereof, selected from the group comprising ZNF671, ZNF776, ZNF586, ZNF552, ZNF587B, ZNF417, ZNF107, ZNF121, ZNF200, ZNF239, ZNF473, ZNF547, ZNF558, ZNF587, ZNF695, ZNF7, ZNF700, ZNF749, ZNF766, ZNF783, ZNF789, ZNF814, ZNF841, and ZNF850, is detected and/or measured.

43. The method of any one of claims 40 to 42, wherein the cancer subject is determined as having a poor prognosis when an alteration of the transcription and/or expression and/or activity level(s) of one or more KZFP, variant or fragment thereof, selected from the group comprising ZNF107, ZNF121, ZNF200, ZNF239, ZNF473, ZNF547, ZNF558, ZNF587, ZNF695, ZNF7, ZNF700, ZNF749, ZNF766, ZNF783, ZNF789, ZNF814, ZNF841, ZNF8-ERVK3-1 and ZNF850 is detected and/or measured.

44. The method of any of claims 40 to 43, wherein the cancer subject is determined as having a poor prognosis when an alteration of the transcription and/or expression and/or activity level(s) of one or more KZFP, variant or fragment thereof, selected from the group comprising ZNF121, ZNF200, ZNF473, ZNF547, ZNF587, ZNF7, ZNF749, ZNF766, ZNF814, and ZNF850 is detected and/or measured.

45. The method of any of claim 40, wherein the cancer subject is suffering from lymphoma and is determined as having a poor prognosis when the transcription and/or expression and/or activity level(s) of one or more KZFP selected from the group comprising ZNF200, ZNF671, ZNF586, ZNF343, ZNF473, ZNF614, ZNF7, ZNF547, ZNF773, ZNF776, ZKSCAN2, ZNF761, ZNF577, ZNF160, ZNF584, ZNF417, ZNF543, ZNF713, ZNF552, ZFP82, ZNF320, ZNF749, ZNF17, ZNF567, ZNF766, ZNF765, ZNF252P, ZNF420, ZNF615, ZNF121, ZNF544, ZNF583, ZNF587, ZNF814, ZNF805, ZNF134, ZNF850, and ZNF587B, variant or fragment thereof, is upregulated.

46. The method of any of claims 40 to 45, wherein the cancer subject is suffering from lymphoma and is determined as having a poor prognosis when the transcription and/or expression and/or activity level(s) of one or more KZFP selected from the group comprising ZNF121, ZNF200, ZNF473, ZNF547, ZNF587, ZNF7, ZNF749, ZNF766, ZNF814, and ZNF850, variant or fragment thereof, is upregulated.

47. The method of any of claims 40 to 46, wherein the lymphoma is aggressive non-Hodgkin lymphoma (NHL).

48. One or more oligonucleotide probes or primers specific to one or more target sequences within an mRNA encoding a KZFP, or a combination of KZFP, selected from any of SEQ ID Nos of Table 1.
